(19) United States
(12) Patent Application Publication
(10) Pub. No.: US 2024/0335384 A1
(43) Pub. Date: Oct. 10, 2024

(54) NITROGEN-CONTAINING CATIONIC LIPID AND APPLICATION THEREOF

(71) Applicant: XIAMEN SINOPEG BIOTECH CO., LTD., Xiamen, Fujian (CN)

(72) Inventors: Sheng LIN, Xiamen (CN); Minggui LIN, Xiamen (CN); Ailan WANG, Xiamen (CN); Linlin WANG, Xiamen (CN); Wengui WENG, Xiamen (CN); Chao LIU, Xiamen (CN); Jinchun YUAN, Xiamen (CN); Qian LIN, Xiamen (CN)

(73) Assignee: XIAMEN SINOPEG BIOTECH CO., LTD., Xiamen, Fujian (CN)

(21) Appl. No.: 18/700,022

(22) PCT Filed: Oct. 13, 2022
(86) PCT No.: PCT/CN2022/125227

§ 371 Date: Apr. 10, 2024

(30) Foreign Application Priority Data
Oct. 15, 2021 (CN) 202111204843.X
Nov. 26, 2021 (CN) 202111424392.0

(51) Int. Cl. A61K 9/127; A61K 45/00; A61K 47/18; C07C 217/08; C07C 219/06; C07C 229/10; C07C 229/26; C07C 235/10

(52) U.S. Cl. CPC A61K 9/1272; A61K 45/00; A61K 47/18; C07C 217/08; C07C 219/06; C07C 229/10; C07C 229/26; C07C 235/10

(57) ABSTRACT

A novel cationic lipid having a structure as represented by general formula (1), which specifically relates to a nitrogen-containing cationic lipid, and also relates to a liposome containing the cationic lipid, a liposome pharmaceutical composition containing said cationic lipid, preparation and application thereof. A cationic liposome containing the cationic lipid represented by formula (1), can improve the loading rate and transport rate of drugs, particularly nucleic acid drugs. The terminus of the novel cationic lipid can contain a fluorescent group or a targeting group, so that the cationic liposome pharmaceutical composition containing the cationic lipid can have fluorescent or targeting function. A formulation of cationic liposome pharmaceutical composition containing nucleic acid exhibiting a very good gene complexing ability and high gene transfection ability, thereby further improves the gene therapeutic and/or gene diagnostic effect of the drug, and provides more selectable cationic lipids in the field of drug delivery.

### CROSS-REFERENCE TO RELATED APPLICATIONS

This application is a U.S. National Phase application of International Application No. PCT/CN2022/125227, filed Oct. 13, 2022, which claims priority to Chinese Application Nos. 202111204843.X, filed Oct. 15, 2021, 202111424392.0, filed Nov. 26, 2021.

### TECHNICAL FIELD

The present invention relates to the field of drug delivery, especially to a cationic lipid as pharmaceutical carrier, in particular to a nitrogen-containing cationic lipid, liposomes containing the cationic lipid, liposome-nucleic acid pharmaceutical compositions containing the cationic liposome, formulations of the compositions and application thereof.

### BACKGROUND OF THE INVENTION

Liposomes are widely used to deliver nucleic acid drugs, genetic vaccines, antitumor drugs, small molecule drugs, polypeptide drugs, or protein drugs. Especially with the approval of two transcriptional messenger RNA (mRNA) vaccines for the prevention of COVID-19, the lipid nanoparticle (LNP) used for loading mRNA has become a popular delivery technology nowadays. In addition to negatively charged mRNA, LNPs contain other four components: ionizable lipids, neutral co-lipids, steroid lipids, and PEGylated lipids, wherein cationic lipids interact with negatively charged mRNA via electrostatic interaction; co-lipids, generally phospholipids, play a role in preventing lipid oxidation, attaching ligands to the surface of the liposome, or reducing the aggregation of lipid particles; steroid lipids with strong membrane fusion properties promote the intracellular uptake and cytoplasmic entry of mRNA, and PEGylated lipids located on the surface of lipid nanoparticles improve the hydrophilicity, avoid rapid clearance by the immune system, prevent particle aggregation, and increase the stability.

Among the four lipids used to prepare LNPs, the most critical is the ionizable cationic lipid, which is non-ionizable and neutrally charged under physiological conditions, and ionizable and partially positively charged under acidic conditions. For example, when the cationic lipids are used as carriers to deliver the nucleic acid drugs, the cationic lipids are encapsulated into the LNPs by binding with nucleic acids (e.g., mRNAs coding the antigens or fluorescent proteins) via electrostatic interaction at a low pH, and the encapsulated LNPs maintain neutral surface charge outside the cell to reduce nonspecific interactions and enter the cell. After entering the cell, the acidic environment inside the cell will change the surface charge of LNPs to be positive, which promotes the escape of mRNA from the lipid nanoparticle to the cytoplasm, where the mRNA will be further translated into corresponding active molecules (e.g., antigens or fluorescent proteins), and finally realizing efficient delivery and transfection of mRNA molecules.

Although cationic lipids have made the latest progress in drug delivery, there is still a need for improved cationic lipids that are suitable for regular therapeutic uses in this field. Literature WO2021026358A1 reports that nitrogen-containing lipids can be protonated and positively or partially positively charged at physiological pH. Therefore, the present invention designs some novel cationic lipids containing nitrogen or multiple nitrogen-branchings.

### SUMMARY OF THE INVENTION

The invention provides novel cationic lipids, cationic liposomes containing the lipids, pharmaceutical compositions containing the cationic liposomes and formulations thereof. The formulations of cationic liposome pharmaceutical compositions could deliver drugs into cells and improve the transport efficiency of drugs, thereby improving the treatment effect of nucleic acid drugs.

The above-described purposes of this invention can be realized via embodiments below.

In one embodiment:

A cationic lipid, wherein its structure is represented by the following general formula (1):

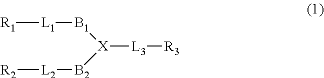

wherein, X is N or CRₐ, said Rₐ is a hydrogen atom or a C₁₋₁₂ alkyl group;
L₁ and L₂ are each independently selected from the group consisting of a linking bond, —OC(═O)—, —C(═O)O—, —OC(═O)O—, —C(═O)—, —O—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—; wherein, R_{c} is, at each occurrence, independently a hydrogen atom or a C₁₋₁₂ alkyl group, s is 2, 3 or 4;
L₃ is a linking bond or a divalent linking group;
B₁ and B₂ are each independently a linking bond or a C₁₋₃₀ alkylene group;
R₁ and R₂ are each independently represented by

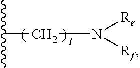

a C₁₋₃₀ aliphatic hydrocarbon group or a residue of C₁₋₃₀ aliphatic hydrocarbon derivative, and at least one of R₁ and R₂ is represented by

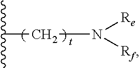

wherein t is an integer from 0 to 12, Rₑ and R_{f} are each independently selected from the group consisting of a C₁₋₁₅ alkyl group, a C₂₋₁₅ alkenyl group, and a C₂₋₁₅ alkynyl group;
R₃ is a hydrogen atom, —R_{d}, —OR_{d}, —NR_{d}R_{d}, —SR_{d}, —C(═O)R_{d}, —C(═O)OR_{d}, —OC(═O)R_{d}, —OC(═O)OR_{d}, or

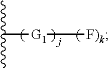

wherein, R_{d} is, at each occurrence, independently a C₁₋₁₂ alkyl group, and two R_{d} in NR_{d}R_{d} can be joined to form a ring; G₁ is a terminal branching group with the valence of k+1; j is 0 or 1; F contains a functional group R₀₁; when j is 0, G₁ is absent; when j is 1, G₁ protrudes F with the number of k, and k is an integer from 2 to 8;
said alkyl group, alkylene group, aliphatic hydrocarbon group, alkenyl group, and alkynyl group are each independently substituted or unsubstituted.

In another embodiment:

Provided herein is a cationic liposome containing cationic lipids represented by general formula (1).

In another embodiment:

Provided herein is a liposome pharmaceutical composition containing cationic liposomes and drugs, said cationic liposomes containing cationic lipids represented by general formula (1).

In another embodiment:

Provided herein is a formulation of liposome pharmaceutical composition, which contains the aforementioned liposome pharmaceutical composition and pharmaceutically acceptable diluents or excipients.

Compared with the Prior Art, the Present Invention Brings the Following Beneficial Effects:

The novel cationic lipid compound of the present invention is a cationic lipid containing a plurality of nitrogen atoms, which enriches the types of cationic lipid and provides more choices for the lipid delivery material, specifically for those applied to the delivery of nucleic acid drugs, genetic vaccines, antitumor drugs, small molecule drugs, polypeptide drugs, protein drugs, and the like, so as to improve the therapeutic and/or diagnostic effects of these drugs as preventive and/or therapeutic agents. The terminal end of the novel cationic lipid of the present invention may also contain a fluorescent group or a targeting group so that the cationic liposome pharmaceutical composition containing the cationic lipid can have the fluorescent or targeting function, which further improves the therapeutic and/or diagnostic effect of the drug, especially when applied to the delivery of the nucleic acid drug, those terminal groups improve the gene therapeutic and/or gene diagnostic effect of the drug.

The novel cationic lipids of the present invention can have a hydrophobic aliphatic tail chain protruded by an amine in a carbamate bond as nitrogen-branching, and the cationic lipid with a hydrophobic aliphatic tail chain protruded by an amine in a carbamate bond as nitrogen-branching at one end and a hydrophobic tail chain protruded by a branched carbon at the other end is the best in terms of encapsulation efficiency and transfection efficiency.

### DETAILED DESCRIPTION OF THE INVENTION

Unless otherwise indicated, the related terms in the present invention are defined as follows.

In the present invention and unless otherwise specified, a structure with isomers may refer to any form of the isomers. For example, when cis- and trans-isomers are present, it can refer to either a cis-structure or a trans-structure; when E and Z isomers are present, it can refer to either an (E)-structure or a (Z)-structure; and when optical rotation properties are present, it can refer to either laevoisomer or dextroisomer.

In the present invention, the definition of the numerical interval includes both the numerical interval indicated by a dash (e.g., 1-6) and the numerical interval indicated by a wavy line (e.g., 1˜6). In the present invention, an integer interval denoted as an interval can represent the group consisting of all integers within the range of the interval unless otherwise specified, and said range includes two endpoints as well. For example, the integer interval 1-6 represents the group consisting of 1, 2, 3, 4, 5, and 6. The numerical interval in the present invention includes but is not limited to the numerical intervals represented by integers, non-integers, percentages and fraction, and all of the foregoing numerical intervals include two endpoints unless otherwise specified.

In the present invention, “formula (2-39) to formula (2-48)” represents the group consisting of formula (2-39), formula (2-40), formula (2-41), formula (2-42), formula (2-43), formula (2-44), formula (2-45), formula (2-46), formula (2-47), and formula (2-48). In the present invention, a numerical value described with “about” or “approximately” generally indicates a numerical range of ±10% which, in some cases, can be amplified to ±15%, not exceeding ±20%, based on the preset numerical value. For example, provided that the molar percentage of steroid lipids in the total lipids in a solvent-containing solution is about 40%, it is generally considered that the molar percentage of steroid lipids is 30%-50%.

In the present invention, unless otherwise specified, the terms “include”, “involve”, “contain”, and similar expressions in the description and claims shall be interpreted as “include but are not limited to”, openly and inclusively.

In the present invention, when two or more objects are “preferably each independently selected from” something and there are multiple levels of preferred options, the objects are not necessarily selected from the preferred options of the same level but allowed to be any of the following cases: one is selected from a wider range of options while the another is selected from a narrower range of options, one is selected from the maximum range of options while another is selected from any allowable options, or all of the objects are selected from the preferred options of the same level.

In the present invention, a divalent linking group, e.g., a hydrocarbylene group, an alkylene group, an arylene group, an amide bond, and the like, can use either one of its two connecting ends to be connected to another group, unless otherwise specified. For example, when an amide bond is used as a divalent linking group between C—CH₂CH₂— and —CH₂-D, both C—CH₂CH₂—C(═O)NH—CH₂-D and C—CH₂CH₂—NHC(═O)—CH₂-D are allowable.

In the present invention, when a terminal group of a linking group in a structural formula is easily confused with a substituent group of said linking group, “ [symbol] ” is used to mark the location where the linking group is connected to other groups. For example, in the structural formulas

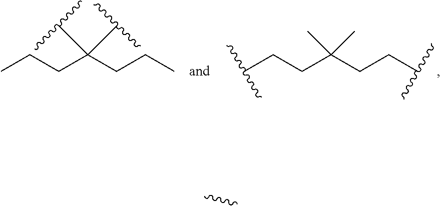

are used to mark the two locations where the divalent linking group is connected to other groups; the two aforementioned structural formulas represent —CH(CH₂CH₂CH₃)₂— and —CH₂CH₂CH(CH₃)₂—CH₂CH₂—, respectively.

In the present invention, the range of the number of carbon atoms in a group is denoted as a subscript of C, representing the number of carbon atoms in the group. For example, C₁₋₁₂ represents a group “having 1 to 12 carbon atoms”, and C₁₋₃₀ indicates a group “having 1 to 30 carbon atoms”. A “substituted C₁₋₁₂ alkyl group” refers to a compound obtained from one or more hydrogen atoms of a C₁₋₁₂ alkyl group being replaced by substituents. A “C₁₋₁₂ substituted alkyl group” refers to a compound having 1 to 12 carbon atoms which is obtained from one or more hydrogen atoms of an alkyl group being replaced by substituents. As another example, when a group can be selected from C₁₋₁₂ alkylene groups, it can be any alkylene group with the number of carbon atoms in the range indicated by the subscript, that is, the group can be selected from the group consisting of C₁, C₂, C₃, C₄, C₅, C₆, C₇, C₈, C₉, C₁₀, C₁₁ and C₁₂ alkylene groups. In the present invention, subscript marked in interval form represents any integer within the range that could be available unless otherwise specified, and the numerical range includes two endpoints.

In the present invention, heteroatoms are not particularly limited, including but not limited to O, S, N, P, Si, F, Cl, Br, I, B, etc.

In the present invention, the heteroatom used for substitution is referred to as “substituent atom”, and the group used for substitution is referred to as “substituent group”.

In the present invention, the term “substituted” indicates that at least one hydrogen atom of any group (e.g., aliphatic hydrocarbon groups, hydrocarbon groups, alkyl groups, or alkylene groups) is replaced by a bond connected to a non-hydrogen atom, said non-hydrogen atom includes but is not limited to a halogen atom, e.g., F, Cl, Br, or I, an oxo group (═O), a hydroxyl group (—OH), a hydrocarbyloxy group (—OR_{d}, wherein R_{d} is a C₁₋₁₂ alkyl group), a carboxyl group (—COOH), an amine group (—NR_{c}R_{c}, wherein both R_{c} are each independently a hydrogen atom or a C₁₋₁₂ alkyl group), a C₁₋₁₂ alkyl group, and a cycloalkyl group. In some embodiments, said substituent group is a C₁₋₁₂ alkyl group. In another embodiment, said substituent group is a cycloalkyl group. In another embodiment, said substituting group is a halide group, e.g., fluoride. In another embodiment, said substituent group is an oxo group. In another embodiment, said substituent group is a hydroxyl group. In another embodiment, said substituent group is an alkoxy group. In another embodiment, said substituent group is a carboxyl group. In another embodiment, said substituent group is an amine group.

In the present invention, a “carbon chain linking group” refers to the linking group whose main chain atoms are all carbon atoms, while the pendant chains are allowed to contain heteroatoms or heteroatom-containing groups that replace the hydrogen atoms connected to the main chain carbon atoms. When a “main chain atom” is a heteroatom, it can also be termed a “main chain heteroatom”. For example, A-S—CH₂—B, A-O—CH₂—B and

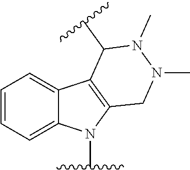 [SMILES: *C1c2c(n(*)c3ccccc23)CN(C)N1C]

(wherein the atomic spacing is 4) are considered to contain main chain heteroatoms. Carbon chain linking groups can be divided into hydrocarbylene groups and carbon chain linking groups containing heteroatoms in their pendant groups; said carbon chain linking group whose pendant groups contain heteroatoms include but are not limited to an oxo group (═O), a thioxo group (═S), an imino group (connected to the main chain carbon through a carbon-nitrogen double bond), an oxa-hydrocarbon group in the form of an ether bond, a thia-hydrocarbon group in the form of a thioether bond, an aza-hydrocarbon group in the form of a tertiary amino group, etc. The main chain of the “carbon chain linking group” is entirely composed of carbon atoms, and the pendant groups of the carbon chain are allowed to contain heteroatoms, that is, the main chain is constructed by connecting methylene groups or substituted methylene groups. Said substituted methylene group can be substituted by one monovalent substituent, two monovalent substituents, or one divalent substituent (e.g., a divalent oxygen atom, or one that forms a three-membered ring

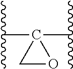 [SMILES: *C1(*)CO1]

with a divalent methylene group). Said substituted methylene group can be a methylene group with one hydrogen atom being substituted (e.g., —CH(CH₃)—), two hydrogen atoms being substituted respectively (e.g., —(CH₃)C(OCH₃)—), or two hydrogen atoms being substituted at the same time (e.g., a carbonyl group, a thiocarbonyl group, —C(═NH)— and —C(═N+H₂)—, or a cyclic pendant group (e.g.,

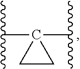 [SMILES: *C1(*)CC1]

wherein the atomic spacing is 1)).

In the present invention, the secondary amino bond and hydrazine bond refers to “—NH—” capped with hydrocarbylene groups at both ends, e.g., —CH₂—NH—CH₂—; while —C(═O)—NH— is termed an amide bond instead of containing a secondary amine bond.

In the present invention, a compound, a group, or an atom can be substituted and heterosubstituted at the same time, for example, a hydrogen atom can be replaced by a nitrophenyl group, and —CH₂—CH₂—CH₂— can be replaced by —CH₂—S—CH(CH₃)—.

In the present invention, a “linking bond” is only for connection and contains no atoms. If the definition of a group includes a linking bond, it means the group can be absent.

In the present invention, the expression “at each occurrence, independently” not only means that different groups can be each independently selected from the definitions but also means that the same group appearing at each different position can also be independently selected from the definition. For example, in —Z-L₄-Z—, “Z is, at each occurrence, independently selected from the group consisting of —C(═O)—, —OC(═O)—, —C(═O)O—, —OC(═O)O—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently a hydrogen atom or a C₁₋₁₂ alkyl group”, in the group “—Z-L₄-Z—”, two Z groups can be the same or different, in the group “—NR_{c}C(═O)NR_{c}—”, two R_{c} groups can be the same or different, which are each independently a hydrogen atom or a C₁₋₁₂ alkyl group.

In the present invention, the term “group” contains at least one atom, referring to the radical formed by a compound losing one or more atoms. Relative to a compound, the group formed by a compound losing part of its groups is also termed as a residue. The valence of a group is not particularly limited, and examples include a monovalent group, a divalent group, a trivalent group, a tetravalent group, . . . , a hectovalent group, etc. Wherein, groups whose valence is equal to or greater than two are collectively defined as linking groups. A linking group can also contain only one atom, such as an oxygen group and a sulfur group.

In the present invention, the term “hydrocarbon” refers to a class of compounds that contains only carbon atoms and hydrogen atoms.

In the present invention, hydrocarbons are classified into aliphatic hydrocarbons and aromatic hydrocarbons by category. Hydrocarbons containing neither phenyl rings nor hydrocarbyl-substituted phenyl rings are defined as aliphatic hydrocarbons. Hydrocarbons containing at least one phenyl ring or hydrocarbyl-substituted phenyl ring are defined as aromatic hydrocarbons. An aromatic hydrocarbon can contain aliphatic hydrocarbon groups, such as toluene, diphenylmethane, 2,3-dihydroindene, etc.

In the present invention, hydrocarbons are classified into saturated hydrocarbons and unsaturated hydrocarbons by the degree of saturation. All aromatic hydrocarbons are unsaturated hydrocarbons. Saturated aliphatic hydrocarbons are also termed alkanes. The degree of unsaturation of unsaturated aliphatic hydrocarbons is not particularly limited. For example, it includes but is not limited to alkenes (containing carbon-carbon double-bonds), alkynes (containing carbon-carbon triple-bonds), dienes (containing two conjugated carbon-carbon double-bonds), and the like. When the aliphatic moieties are saturated in an aromatic hydrocarbon, said aromatic hydrocarbon is also termed arylalkane, such as toluene.

In the present invention, the structures of hydrocarbons are not particularly limited, including linear structures without pendant groups, branched structures bearing pendant groups, cyclic structures containing at least one ring, dendritic structures, comb-like structures, hyperbranched structures, etc. Unless otherwise specified, preferable structures include linear structures without pendant groups, branched structures bearing pendant groups, and cyclic structures, which correspond to linear hydrocarbons, branched hydrocarbons, and cyclic hydrocarbons, respectively. Wherein, hydrocarbons without cyclic structures are termed open-chain hydrocarbons, including but not limited to linear structures without pendant groups, and branched structures bearing pendant groups. Open-chain hydrocarbons fall into the scope of aliphatic hydrocarbons, so linear hydrocarbons are also described as linear aliphatic hydrocarbons and branched hydrocarbons are also described as branched aliphatic hydrocarbons.

In the present invention, hydrocarbons with any carbon atom replaced by a heteroatom are generally referred to as hetero-hydrocarbons.

In the present invention, “hydrocarbon group” refers to the residue of a hydrocarbon molecule with at least one hydrogen atom removed. According to the number of lost hydrogen atoms, hydrocarbon groups can be classified into monovalent hydrocarbon groups (with one hydrogen atom lost), divalent hydrocarbon groups (with two hydrogen atoms lost, which is also referred to as hydrocarbylene group), trivalent hydrocarbon groups (with three hydrogen atoms lost), and the like. Accordingly, when n hydrogen atoms are lost, the valence of the resulting hydrocarbon group is n. In the present invention, hydrocarbon groups refer specifically to monovalent hydrocarbon groups, unless otherwise specified.

In the present invention, the source of hydrocarbon group is not particularly limited, for example, aliphatic hydrocarbons or aromatic hydrocarbons, saturated hydrocarbons or unsaturated hydrocarbons, linear hydrocarbons, branched hydrocarbons or cyclic hydrocarbons, hydrocarbons or heterohydrocarbons, etc. According to the degree of saturation, e.g., the source can be alkanes, alkenes, alkynes, dienes, etc.; with respect to cyclic hydrocarbons, the source can be, e.g., alicyclic hydrocarbons or aromatic hydrocarbons, monocyclic hydrocarbons or polycyclic hydrocarbons; and with respect to heterocyclic hydrocarbons, the source can be, e.g., aliphatic heterocyclic hydrocarbons or aromatic heterocyclic hydrocarbons.

In the present invention, the “aliphatic hydrocarbon group” refers to the residue of an aliphatic hydrocarbon molecule with at least one hydrogen atom removed. Aliphatic hydrocarbon groups in the present invention refer specifically to monovalent aliphatic hydrocarbon groups, unless otherwise specified. Aliphatic hydrocarbon groups include saturated aliphatic hydrocarbon groups and unsaturated aliphatic hydrocarbon groups.

In the present invention, the “alkyl group” refers to a hydrocarbon group obtained from an alkane losing a hydrogen atom at any location, unless otherwise specified. Said alkyl group can be linear or branched, substituted or unsubstituted. Specific examples include that a propyl group refers to either a 1-propyl group or an isopropyl group, and a propylene group refers to a 1,3-propylene group, a 1,2-propylene group, or an isopropylidene group.

In the present invention, the “unsaturated hydrocarbon group” refers to a hydrocarbon group obtained from an unsaturated hydrocarbon losing hydrogen atoms. The hydrocarbon group obtained from an unsaturated hydrocarbon losing hydrogen atoms bonded to unsaturated carbon atoms can be an alkenyl group, an alkynyl group, or a dienyl group, etc., e.g., a propenyl group or a propynyl group. According to the difference of unsaturated bonds, the hydrocarbon group obtained from an unsaturated hydrocarbon losing hydrogen atoms bonded to saturated carbon atoms can be termed, e.g., an alkenyl hydrocarbon group, analkyne, a dienyl hydrocarbon group, or the like, and specifically, e.g., an allyl group or a propargyl group.

In the present invention, the “alkenyl” or “alkenyl group” refers to a substituted or unsubstituted, linear or branched alkenyl group which contains two or more carbon atoms (e.g., two, three, four, five, six, seven, eight, nine, ten, eleven, twelve, thirteen, fourteen, fifteen, sixteen, seventeen eighteen, nineteen, twenty or more carbon atoms) and at least one carbon-carbon double bond. The term “C₂₋₁₅ alkenyl group” refers to a substituted or unsubstituted, linear or branched alkenyl group which contains 2-15 carbon atoms and at least one carbon-carbon double bond, that is, an alkenyl group can contain one, two, three, four, or more carbon-carbon double bonds. Alkenyl groups in the present invention include substituted and unsubstituted alkenyl groups unless otherwise specified.

In the present invention, the “alkynyl” or “alkynyl group” refers to a linear or branched, optionally substituted hydrocarbon which contains two or more carbon atoms (e.g., two, three, four, five, six, seven, eight, nine, ten, eleven, twelve, thirteen, fourteen, fifteen, sixteen, seventeen eighteen, nineteen, twenty or more carbon atoms) and at least one carbon-carbon triple bond. The term “C₂₋₁₅ alkynyl group” refers to a substituted or unsubstituted, linear or branched alkynyl group which contains 2-15 carbon atoms and at least one carbon-carbon triple bond, that is, an alkynyl group can contain one, two, three, four, or more carbon-carbon triple bonds. Alkynyl groups in the present invention include substituted and unsubstituted alkynyl groups unless otherwise specified.

In the present invention, the aliphatic hydrocarbon derivatives are preferably ether derivatized aliphatic hydrocarbon and aliphatic hydrocarbon derivatives containing 1-2 ether bonds, more preferably aliphatic hydrocarbon derivatives containing 2 ether bonds.

In the present invention, the term “molecular weight” represents the mass of a compound, and unless otherwise specified, the unit of measurement of “molecular weight” is Dalton, Da.

In the present invention, concerning the percentage, “about” and “approximately” generally refer to a tolerance of 0.5%.

In the present invention, the terms “stable group” and “degradable group” are a pair of opposing concepts, the detailed examples of stable groups and degradable groups can be found in paragraphs [0134]-[0145] of CN113402405A.

In the present invention, the term “hydroxyl protecting group” includes all the groups which can be used as common hydroxyl protecting groups. A hydroxyl protecting group is preferably selected from the group consisting of an alkanoyl group (e.g., an acetyl group and a butyryl group), an aromatic alkanoyl group (e.g., a benzoyl group), a benzyl group, a triphenylmethyl group, a trimethylsilyl group, a t-butyldimethylsilyl group, an allyl group, an acetal group, and a ketal group. The removal of an acetyl group is generally carried out under alkaline conditions, most commonly by the ammonolysis with NH₃/MeOH or by the methanolysis catalyzed by methanol anions. The benzyl group is easy to be removed via palladium-catalyzed hydrogenolysis in a neutral solution at room temperature, or via the reduction reaction with metallic sodium in ethanol or liquid ammonia. The triphenylmethyl group is usually removed by catalytic hydrogenolysis. The trimethylsilyl group is usually removed with reagents containing fluoride ions (e.g., tetrabutylammonium fluoride/anhydrous THF, etc.). The t-butyldimethylsilyl ether is relatively stable, which can withstand the ester hydrolysis with alcoholic potassium hydroxide and mild reduction conditions (e.g., Zn/CH₃OH and the like), and can be removed by fluoride ions (e.g., Bu₄N⁺F⁻) in THE solution or by aqueous acetic acid at room temperature.

In the present invention, the “carboxyl protecting group” refers to the protecting group which can be transformed into a carboxyl group through hydrolysis or the deprotection reaction of the carboxyl protecting group itself. A carboxyl protecting group is preferably selected from the group consisting of an alkyl group (e.g., a methyl group, an ethyl group and a butyl group) and an aralkyl group (e.g., a benzyl group), and more preferably selected from the group consisting of a butyl group (tBu), a methyl group (Me), and an ethyl group (Et). In the present invention, the “protected carboxyl group” refers to the group obtained from a carboxyl group being protected by an appropriate carboxyl protecting group, preferably selected from the group consisting of a methoxycarbonyl group, an ethoxycarbonyl group, a t-butyloxycarbonyl group, and a benzyloxycarbonyl group. Said carboxyl protecting group can be removed through hydrolysis catalyzed by acids or alkalis, or through pyrolysis reaction occasionally; for example, t-butyl groups can be removed under mild acidic conditions, and benzyl groups can be removed by hydrogenolysis. The reagent used for removing carboxyl protecting groups is selected from the group consisting of TFA, H₂O, LiOH, NaOH, KOH, MeOH, EtOH and combinations thereof, preferably selected from the combination of TFA and H₂O, the combination of LiOH and MeOH, and the combination of LiOH and EtOH. The deprotection of a protected carboxyl group can produce the corresponding free acid, which can be carried out in the presence of an alkali that forms pharmaceutically acceptable salts with said free acid generated during said deprotection.

In the present invention, the term “amino protecting group” includes all the groups which can be used as common amino protecting groups, such as an aryl C₁₋₆ alkyl group, a C₁₋₆ alkoxy C₁₋₆ alkyl group, a C₁₋₆ alkoxycarbonyl group, an aryloxycarbonyl group, C₁₋₆ alkylsulfonyl group, an arylsulfonyl group, a silyl group, etc. An amino protecting group is preferably selected from the group consisting of a t-butoxycarbonyl group (Boc), a p-methoxybenzyloxycarbonyl group (Moz) and a 9-fluorenylmethoxycarbonyl group (Fmoc).

The reagent used for removing amino protecting groups is selected from the group consisting of TFA, H₂O, LiOH, MeOH, EtOH and combinations thereof, preferably selected from the combination of TFA and H₂O, the combination of LiOH and MeOH, and the combination of LiOH and EtOH. The reagent used for removing the Boc protecting group is TFA or HCl/EA, preferably TFA. The reagent used for removing the Fmoc protecting group is the N,N-dimethylformamide (DMF) solution containing 20% piperidine.

In the present invention, the “activation of a carboxyl group” refers to the activation of a carboxyl group with carboxyl activating agents. The activated carboxyl group can promote condensation reactions, for example, by inhibiting the generation of racemic impurities, by accelerating the reaction through catalysis, etc. The “carboxyl activating group” refers to the residue of a carboxyl activating agent. Said carboxyl activating agent is selected from the group consisting of N-hydroxysuccinimide (NHS), 1-ethyl-3-(3-dimethylaminopropyl)carbodiimide hydrochloride (EDCI), N-hydroxy-5-norbornene-2,3-dicarboximide (HONb), and N,N′-dicyclohexylcarbodiimide (DCC), and combinations thereof, preferably selected from the combination of NHS/EDCI, the combination of NHS/DCC, and the combination of HONb/DCC, and more preferably the combination of NHS/EDCI.

In the present invention, the term “cation” refers to the corresponding structure bearing a positive charge, either permanently, or non-permanently but in response to certain conditions (such as pH). Therefore, the cations include permanent cations and those cationisable compounds, groups, or atoms. Permanent cations refer to the corresponding compounds, groups, or atoms that bear positive charges under conditions of any pH value or hydrogen ion activity of their environment. Typically, a positive charge is generated by the presence of quaternary nitrogen atom. When a compound carries multiple such positive charges, it can be called a permanent cation. A cationisable substance refers to a compound, group or atom that is positively charged at a lower pH and uncharged at a higher pH of its environment. On the other hand, in non-aqueous environments where the pH cannot be determined, a cationisable compound, group, or atom is positively charged at a high hydrogen ion concentration and uncharged at a low concentration or activity of hydrogen ions. It depends on the individual properties of the cationisable or polycationisable compound, especially the pKa of the respective cationisable group or atom, at which pH or hydrogen ion concentration said compound is charged or uncharged. In the diluted aqueous environment, the so-called Henderson-Hasselbalch equation can be used to estimate the fraction of positively charged cationisable compounds, groups, or atoms, which is well-known to those skilled in the art. For example, in some embodiments, if a compound or moiety is cationisable, it is preferred that it is positively charged at a pH value of about 1 to 9, preferably 4 to 9, 5 to 8, or even 6 to 8, more preferably at a pH value of or below 9, of or below 8, of or below 7, and most preferably at physiological pH values (e.g., about 7.3 to 7.4), i.e., under physiological conditions, particularly under physiological salt conditions of the cell in vivo. In other embodiments, it is preferred that the cationisable compound or moiety is predominantly neutral at physiological pH values (e.g., about 7.0 to 7.4), but becomes positively charged at lower pH values. In some embodiments, the preferred range of pKa for the cationisable compound or moiety is about 5 to 7.

In the present invention, “cationic liposome” refers to the liposome which is positively charged or ionizable as a whole. Except for the molecules represented by the general formula (1), cationic liposomes include but are not limited to N,N-dioleyl-N,N-dimethylammonium chloride (DODAC), N,N-distearyl-N,N-dimethylammonium bromide (DDAB), 1,2-dioleoyl-3-trimethylammonium-propane (DOTAP), N-[1-(2,3-dioleoyloxy)propyl]-N,N,N-trimethylammonium chloride (DOTMA) 1,2-dioleyloxy-N,N-dimethylaminopropane (DODMA), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino)octanoate (SM102) and mixtures thereof.

In the present invention, the “PEGylated lipid” refers to a molecule containing lipid and polyethylene glycol moieties. In addition to those represented by the general formula (2) in the present invention, PEGylated lipids also include but are not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-cholesterol, PEG-diacylglycamide (PEG-DAG), PEG-dialkyloxypropyl (PEG-DAA), and specifically PEG500-dipalmitoylphosphatidylcholine, PEG2000-dipalmitoylphosphatidylcholine, PEG500-stearylphosphatidylethanolamine PEG2000-distearylphosphatidylethanolamine, PEG500-1,2-oleoylphosphatidylethanolamine, PEG2000-1,2-oleoylphosphatidylethanolamine, PEG2000-2,3-dimyristoylglycerol (PEG-DMG), and the like.

In the present invention, the “neutral lipid” refers to any lipid substance which is uncharged or exists in the form of neutral zwitterion at the chosen pH, preferably phospholipid. This kind of lipid includes but is not limited to 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dioleoylphosphatidylserine (DOPS), dipalmitoylphosphatidylglycerol (DPPG), palmitoyloleoylphosphatidylethanolamine (POPE), distearoylphosphatidylethanolamine (DSPE), dipalmitoylphosphatidylethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), and combinations thereof. The neutral lipid can be synthetic or natural.

In the present invention, a “steroid lipid” is selected from the group consisting of cholesterol, coprostanol, sitosterol, ergosterol, campesterol, stigmasterol, brassicasterol tomatidine, ursolic acid, α-tocopherol, and mixtures thereof.

In the present invention, an “amino acid residue” is an amino acid from which, formally, a hydrogen atom has been removed from an amino group and/or from which, formally, a hydroxy group has been removed from a carboxy group and/or from which, formally, a hydrogen atom has been removed from a sulfhydryl group and/or with a protected amino group and/or with a protected carboxyl group and/or with a protected sulfhydryl group. Imprecisely, an amino acid residue can be described as an amino acid. The source of the amino acid in the present invention is not particularly limited unless otherwise specified, which can be either natural or unnatural, or a mixture of both. The configuration of the amino acid in the present invention is not particularly limited unless otherwise specified, which can be either L-type or D-type, or a mixture of both.

In the present invention, the term “functional group source” refers to those having reaction activity or potential reaction activity, photosensitivity or potential photosensitivity, targeting properties or potential targeting properties. Said “potential” means that the functional group source can be converted into a reactive group through chemical processes including but not limited to functional modification (e.g., grafting, substitution, etc.), deprotection, salt complexation and decomplexation, ionization, protonation, deprotonation, leaving group transformation, etc., and can exhibit luminescence or targeting properties through external stimuli such as light, heat, enzymes, specific binding molecules, microenvironment in vivo, etc. Said luminescence is not particularly limited, including but not limited to visible light, fluorescence, phosphorescence, etc.

In the present invention, a variant form refers to a structural form that can be transformed into the target reactive group after any process of chemical change selected from the group consisting of oxidation, reduction, hydration, dehydration, electronic rearrangement, structural rearrangement, salt complexation and decomplexation, ionization, protonation, deprotonation, substitution, deprotection, leaving group transformation, etc.

In the present invention, a “variant form of reactive group” refers to a form which remain active (reactive group) after the reactive group undergoes at least one process of chemical change selected from oxidation, reduction, hydration, dehydration, electronic rearrangement, structural rearrangement, salt complexation and decomplexation, ionization, protonation, deprotonation, substitution, deprotection, leaving group transformation, etc., or a non-reactive form that has been protected.

In the present invention, the “micro-modification” refers to a chemical modification process that can be completed through simple chemical reaction processes. Said simple chemical reaction process mainly refers to deprotection, salt complexation and decomplexation, ionization, protonation, deprotonation, leaving group transformation, etc. “micro-variant form” is corresponding to the “micro-modification”, which refers to the structural form that can be transformed into the target reactive group after any process of chemical change selected from the group consisting of deprotonation, salt complexation and decomplexation, ionization, protonation, deprotection, leaving group transformation, etc. Said leaving group transformation is, for example, the conversion of the ester form to the acyl chloride form.

In the present invention, the “N/P ratio” refers to a molar ratio of nitrogen atoms in cationic lipids to phosphoric acid in nucleic acids.

In the present invention, the “nucleic acid” refers to DNA, RNA or a modified form thereof.

In the present invention, the term “RNA” refers to a ribonucleic acid that may be naturally or non-naturally occurring. For example, an RNA may include modified and/or non-naturally occurring components such as one or more nucleobases, nucleosides, nucleotides, or linkers.

An RNA may include a cap structure, a chain-terminating nucleoside, a stem loop, a polyA sequence, and/or a polyadenylation signal. An RNA may have a nucleotide sequence encoding a polypeptide of interest. For example, an RNA may be a messenger RNA (mRNA). The translation of an mRNA encoding a particular polypeptide, for example, the in vivo translation of an mRNA inside a mammalian cell, may produce the encoded polypeptide. An RNA may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, single guide RNA (sgRNA), cas9 mRNA and mixtures thereof.

In the present invention, the Fluc mRNA can express the luciferase protein which emits bioluminescence in the presence of fluorescein substrates, so Fluc is commonly used in mammalian cell culture to measure gene expression and cell activity.

In the present invention, assays for determining the level of target gene expression include but are not limited to dot blots, northern blots, in situ hybridization, ELISA, immunoprecipitation, enzyme function, and phenotypic assays.

In the present invention, the term “transfection” refers to the introduction of a species (e.g., RNA) into a cell. Transfection may occur, for example, in vitro, ex vivo, or in vivo.

In the present invention, the term “antigen” typically refers to a substance that can be recognized by the immune system, preferably recognized by the adaptive immune system, and triggers an antigen-specific immune response, for example, forming antibodies and/or antigen-specific T cells as a part of the adaptive immune response. Typically, the antigen may be or may contain a peptide or a protein that can be presented to T cells by MHC. In the present invention, the antigen may be a translation product of the provided nucleic acid molecule (preferably mRNA as defined herein). In this context, fragments, variants, and derivatives of peptides and proteins containing at least one epitope are also recognized as antigens.

In the present invention, the term “delivery” refers to providing an entity to the target, for example, delivering drugs and/or therapeutic agents and/or prophylactic agents to subjects, said subjects being tissues and/or cells of humans and/or other animals.

In the present invention, the “pharmaceutically acceptable carrier” refers to a diluent, adjuvant, excipient, or vehicle administered together with the therapeutic agent, which is, in a reasonable medical judgment range, suitable for the contact of the human and/or other animals without excessive toxicity, stimulation, hypersensitive response, other problems or complications corresponding to a reasonable benefit/risk ratio. Pharmaceutically acceptable carriers that can be used in the pharmaceutical composition in the present invention include but are not limited to sterile liquids, e.g., water and oil, including the oil from petroleum, animal, plant, or synthetic origin, e.g., peanut oil, soybean oil, mineral oil, sesame oil, etc. When said pharmaceutical composition is administered intravenously, water is an exemplary carrier. Physiological saline, glucose and aqueous glycerol solution can also be used as liquid carriers, and are especially used for injection. Suitable pharmaceutical excipients include starch, glucose, lactose, sucrose, gelatin, maltose, chalk, silica gel, sodium stearate, glyceryl monostearate, talc, sodium chloride, defatted milk powder, glycerol, propylene glycol, water, ethanol, etc. Said composition can also contain a small amount of humectant, emulsifier, or pH buffer as needed. Oral preparations can contain standard carriers, e.g., pharmaceutical grade mannitol, lactose, starch, magnesium stearate, sodium saccharin, cellulose, magnesium carbonate, etc. Specifically, excipients include but are not limited to anti-adherents, antioxidants, binders, coatings, compression aids, disintegrants, dyes (pigments), emollients, emulsifiers, fillers (diluents), film formers or coatings, flavors, fragrances, glidants (flow enhancers), lubricants, preservatives, printing inks, sorbents, suspending or dispersing agents, sweeteners, and waters for hydration. More specifically, excipients include but are not limited to butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, phenyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (α-tocopherol), vitamin C, and xylitol.

In the present invention, pharmaceutical compositions can act systemically and/or locally. For this purpose, they can be administered by appropriate routes such as injection (e.g., intravenous, intraarterial, subcutaneous, intraperitoneal, or intramuscular injection, including instillation) or transdermal administration, or administered by oral, buccal, transnasal, transmucosal or topical routes or in the form of ophthalmic preparation or by inhalation. Regarding these administration routes, the pharmaceutical compositions of the present invention can be administered in a suitable dosage form. The dosage forms include but are not limited to tablets, capsules, lozenges, hard sugar agents, powders, sprays, creams, ointments, suppositories, gels, pastes, emulsions, ointments, aqueous suspensions, injectable solutions, elixirs, and syrups.

In the present invention, vaccines are preventive or therapeutic materials that provide at least one antigen or antigenic function. An antigen or antigenic function can stimulate the body's adaptive immune system to provide an adaptive immune response.

In the present invention, treatment refers to the management and care of patients to resist diseases, obstacles, or symptoms, which is intended to delay the development of diseases, obstacles, or symptoms, reduce or alleviate symptoms and complications, and/or cure or eliminate diseases, obstacles or symptoms. The patients to be treated are preferably mammals, especially humans.

### DETAILED DISCLOSURE OF THE INVENTION

In one embodiment:

1.1. A cationic lipid, wherein its structure is represented by the general formula (1):

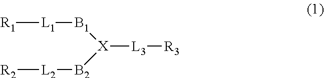

wherein, X is N or CRₐ, said Rₐ is a hydrogen atom or a C₁₋₁₂ alkyl group;
L₁ and L₂ are each independently selected from the group consisting of a linking bond, —OC(═O)—, —C(═O)O—, —OC(═O)O—, —C(═O)—, —O—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—; wherein, R_{c} is, at each occurrence, independently a hydrogen atom or a C₁₋₁₂ alkyl group, s is 2, 3 or 4;
L₃ is a linking bond or a divalent linking group;
B₁ and B₂ are each independently a linking bond or a C₁₋₃₀ alkylene group;
R₁ and R₂ are each independently represented by

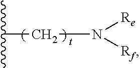

a C₁₋₃₀ aliphatic hydrocarbon group or a residue of C₁₋₃₀ aliphatic hydrocarbon derivative, and at least one of R₁ and R₂ is represented by

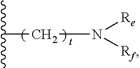

wherein t is an integer from 0 to 12, Rₑ and R_{f} are each independently selected from the group consisting of a C₁₋₁₅ alkyl group, a C₂₋₁₅ alkenyl group, and a C₂₋₁₅ alkynyl group;
R₃ is a hydrogen atom, —R_{d}, —OR_{d}, —NR_{d}R_{d}, —SR_{d}, —C(═O)R_{d}, —C(═O)OR_{d}, —OC(═O)R_{d}, —OC(═O)OR_{d}, or

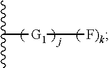

wherein, R_{d} is, at each occurrence, independently a C₁₋₁₂ alkyl group, and two R_{d} in NR_{d}R_{d} can be joined to form a ring; G₁ is a terminal branching group with the valence of k+1; j is 0 or 1; F contains a functional group R₀₁; when j is 0, G₁ is absent; when j is 1, G₁ protrudes F with the number of k, and k is an integer from 2 to 8;
said alkyl group, alkylene group, aliphatic hydrocarbon group, alkenyl group, and alkynyl group are each independently substituted or unsubstituted.

### 1.1.1.X

In the present invention, X is, at each occurrence, independently N or CRₐ, wherein Rₐ is a hydrogen atom or a C₁₋₁₂ alkyl group.

1.1.2. L₁, L₂, L₃, L₄, L₅, L₇, L₈, Z, Z₁, Z₂

In the present invention, the structures of L₁, L₂, L₃, L₄, L₅, L₇, L₈, Z, Z₁, and Z₂ are not particularly limited, each independently including but not limited to linear structures, branched structures, and ring-containing structures.

In the present invention, the number of non-hydrogen atoms of L₁, L₂, L₃, L₄, L₅, L₇, L₈, Z, Z₁, and Z₂ are not particularly limited, each preferably ranging independently from 1 to 50, more preferably from 1 to 20, and more preferably from 1 to 10. Said non-hydrogen atom is a carbon atom or a heteroatom. Said heteroatom includes but is not limited to O, S, N, P, Si, B, etc. When the number of non-hydrogen atoms is 1, it can be a carbon atom or a heteroatom. When the number of non-hydrogen atoms is larger than 1, the species of non-hydrogen atoms are not particularly limited, which can be one species, two or two more species, and any combination of carbon atoms and carbon atoms, of carbon atoms and heteroatoms, or of heteroatoms and heteroatoms.

In the present invention, two identical or different reactive groups may form a divalent linking group after a reaction. The reaction conditions are related to the types of the resulting divalent linking groups, and the prior art can be introduced herein. For example, amino groups can react with active esters, active formates, sulfonate esters, aldehydes, α,β-unsaturated bonds, carboxylic groups, epoxides, isocyanates, and isothiocyanates, respectively, to obtain divalent linking groups such as amide groups, urethane groups, amino groups, imide groups (which can be further reduced to secondary amino groups), amino groups, amide groups, amino alcohols, urea bonds, thiourea bonds, etc.; mercapto groups can react with active esters, active formates, sulfonate groups, mercapto groups, maleimide groups, aldehyde groups, α,β-unsaturated bonds, carboxyl groups, iodoacetamide groups, and anhydride groups, respectively, to obtain divalent linking groups such as thioester groups, thiocarbonate groups, thioether groups, disulfide groups, thioether groups, thiohemiacetal linking groups, thioether groups, thioester groups, thioether groups, imide groups, etc.; unsaturated bonds can react with mercapto groups to obtain thioether groups; carboxyl groups or acyl halides can respectively react with mercapto groups and amino groups to obtain groups such as thioester groups, amide groups, etc.; hydroxyl groups can react with carboxyl groups, isocyanates, epoxides, and chlorocarbonyloxy groups to obtain divalent linking groups such as ester groups, carbamate groups, ether bonds, carbonate groups, etc.; carbonyl groups or aldehyde groups can react with amino groups, hydrazines and hydrazides to obtain divalent linking groups such as imine bonds, hydrazone groups, acylhydrazone groups, etc.; reactive groups such as azido groups, alkynyl groups, alkenyl groups, mercapto groups, azido groups, dienyl groups, maleimide groups, 1,2,4-triazoline-3,5-dione groups, dithioester groups, hydroxylamine groups, hydrazide groups, acrylate groups, allyloxy groups, isocyanate groups, tetrazole groups and the like can undergo click reactions to form various divalent linking groups including but not limited to the structures of triazoles, isoxazoles, thioether bonds, and the like.

The stability of divalent linking groups L₁, L₂, L₃, L₄, L₅, L₇, L₈, Z, Z₁, and Z₂ are not particularly limited, wherein any divalent linking group or a divalent linking group consisting of any aforementioned divalent linking group and adjacent heterosubstituted groups thereof is independently a stable linking group STAG or a degradable linking group DEGG

1.1.2.1. L₁, L₂

In the present invention, L₁ and L₂ are each independently selected from the group consisting of a linking bond, —OC(═O)—, —C(═O)O—, —OC(═O)O—, —C(═O)—, —O—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently, a hydrogen atom or a C₁₋₁₂ alkyl group, s is 2, 3, or 4.

In one specific embodiment of the present invention, L₁ and L₂ are preferably selected from the following situations:
(1): one of L₁ and L₂ is a linking bond, and the other is selected from the group consisting of —OC(═O)—, —C(═O)O—, —OC(═O)O—, —C(═O)—, —O—, —O(CR_{c}R_{c})ₛO—, —S—, —C(═O)S—, —SC(═O)—, and —NR_{c}C(═O)S—;
(2): both L₁ and L₂ are linking bonds;
(3): L₁ and L₂ are each independently selected from the group consisting of —OC(═O)—, —C(═O)O—, —OC(═O)O—, —C(═O)—, —O—, —O(CH₂)ₛO—, —S—, —C(═O)S—, —SC(═O)—, —NHC(═O)—, —C(═O)NH—, —NHC(═O)NH—, —OC(═O)NH—, —NHC(═O)O—, —SC(═O)NH—, and —NHC(═O)S—.

In one specific embodiment of the present invention, more preferably L₁ and L₂ are each independently selected from the group consisting of —OC(═O)—, —C(═O)O—, and —OC(═O)O—.

In one specific embodiment of the present invention, more preferably one of L₁ and L₂ is —C(═O)O—, and the other is —OC(═O)— or —C(═O)O—.

In one specific embodiment of the present invention, more preferably L₁ and L₂ are both —C(═O)O—.

In one specific embodiment of the present invention, R_{c} is preferably a hydrogen atom, or preferably a C₁₋₁₂ alkyl group, more preferably a C₁₋₈ alkyl group, more preferably selected from the group consisting of a methyl group, an ethyl group, a propyl group, a butyl group, a pentyl group, and a hexyl group.

1.1.2.2. L₇, L₈

In the present invention, L₇ and L₈ are each independently a linking bond or a divalent linking group, said divalent linking group is selected from the group consisting of —OC(═O)—, —C(═O)O—, —OC(═O)O—, —C(═O)—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein said R_{c} is, at each occurrence, independently a hydrogen atom or a C₁₋₁₂ alkyl group.

In one specific embodiment of the present invention, R_{c} is preferably a hydrogen atom, or preferably a C₁₋₁₂ alkyl group, more preferably a C₁₋₈ alkyl group, more preferably selected from the group consisting of a methyl group, an ethyl group, a propyl group, a butyl group, a pentyl group, and a hexyl group.

### 1.1.2.3. L₃

In the present invention, L₃ is a linking bond or a divalent linking group.

In one specific embodiment of the present invention, L₃ is a divalent linking group, preferably selected from the divalent linking group formed by any one, two, or two more divalent linking group selected from L₄, L₅, and Z; more preferably selected from the group consisting of -L₄-, —Z-L₄-Z—, -L₄-Z-L₅-, —Z-L₄-Z-L₅-, and -L₄-Z-L₅-Z—; wherein, said L₄ and L₅ are carbon linking groups, which are each independently represented by —(CRₐR_{b})ₜ—(CRₐR_{b})ₒ—(CRₐR_{b})ₚ—, t, o, and p are each independently an integer from 0 to 12, not being 0 simultaneously; Rₐ and R_{b} are, at each occurrence, independently a hydrogen atom or a C₁₋₁₂ alkyl group; said Z is, at each occurrence, independently selected from the group consisting of —C(═O)—, —OC(═O)—, —C(═O)O—, —OC(═O)O—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently a hydrogen atom or a C₁₋₁₂ alkyl group, C₁₋₁₂ alkyl group is substituted or unsubstituted, preferably selected from the group consisting of a methyl group, an ethyl group, a propyl group, a butyl group, a pentyl group, and a hexyl group, a heptyl group, and an octyl group.

In one specific embodiment of the present invention, the R_{c} in the aforementioned L₃ is preferably a hydrogen atom.

In one specific embodiment of the present invention, L₃ is preferably selected from the group consisting of —(CH₂)ₜ—, —(CH₂)ₜZ—, —Z(CH₂)ₜ—, —(CH₂)ₜZ(CH₂)ₜ—, and —Z(CH₂)ₜZ—, wherein t is an integer from 1 to 12, Z is selected from the group consisting of —C(═O)—, —OC(═O)—, —C(═O)O—, —OC(═O)O—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—. L₃ is preferably selected from the group consisting of —(CH₂)—, —(CH₂)ₜO—, —(CH₂)ₜC(═O)—, —(CH₂)C(═O)O—, —(CH₂)ₜOC(═O)—, —(CH₂)C(═O)NH—, —(CH₂)ₜNHC(═O)—, —(CH₂)ₜOC(═O)O—, —(CH₂)ₜNHC(═O)O—, —(CH₂)ₜOC(═O)NH—, —(CH₂)ₜNHC(═O)NH—, —O(CH₂)ₜ—, —C(═O)(CH₂)ₜ—, —C(═O)O(CH₂)—, —OC(═O)(CH₂)ₜ—, —C(═O)NH(CH₂)ₜ—, —NHC(═O)(CH₂)ₜ—, —OC(═O)O(CH₂)ₜ—, —NHC(═O)O(CH₂)—, —OC(═O)NH(CH₂)ₜ—, —NHC(═O)NH(CH₂)ₜ—, —(CH₂)O(CH₂)ₜ—, —(CH₂)C(═O)(CH₂)ₜ—, —(CH₂)C(═O)O(CH₂)ₜ—, —(CH₂)ₜOC(═O)(CH₂)—, —(CH₂)C(═O)NH(CH₂)ₜ—, —(CH₂)ₜNHC(═O)(CH₂)—, —(CH₂)OC(═O)O(CH₂)ₜ—, —(CH₂)ₜNHC(═O)O(CH₂)—, —(CH₂)ₜOC(═O)NH(CH₂)—, —(CH₂)ₜNHC(═O)NH(CH₂)—, —O(CH₂)ₜO—, —C(═O)(CH₂)ₜC(═O)—, —C(═O)O(CH₂)C(═O)O—, —OC(═O)(CH₂)ₜOC(═O)—, —C(═O)O(CH₂)OC(═O)—, —OC(═O)(CH₂)ₜC(═O)O—, —OC(═O)O(CH₂)ₜOC(═O)O—, —C(═O)NH(CH₂)C(═O)NH—, —NHC(═O)(CH₂)ₜNHC(═O)—, —NHC(═O)(CH₂)ₜC(═O)NH—, —C(═O)NH(CH₂)ₜNHC(═O)—, —NHC(═O)O(CH₂)ₜNHC(═O)O—, —OC(═O)NH(CH₂)OC(═O)NH—, —NHC(═O)O(CH₂)ₜOC(═O)NH—, —OC(═O)NH(CH₂)ₜNHC(═O)O—, —NHC(═O)NH(CH₂)ₜNHC(═O)NH—, —C(═O)(CH₂)ₜO—, —C(═O)(CH₂)ₜC(═O)O—, —C(═O)(CH₂)ₜOC(═O)—, —C(═O)(CH₂)OC(═O)O—, —C(═O)(CH₂)ₜNHC(═O)O—, —C(═O)(CH₂)ₜOC(═O)NH—, and —C(═O)(CH₂)ₜNHC(═O)NH—

1.1.3 B₁, B₂

In the present invention, B₁ and B₂ are each independently a linking bond or a C₁₋₃₀ alkylene group.

In one specific embodiment of the present invention, B₁ and B₂ are preferably each independently a linking bond or a C₁₋₂₀ alkylene group; more preferably selected from the following situations:
(1): B₁ and B₂ are each independently a C₁₋₂₀ alkylene group, specifically selected from the group consisting of a methylene group, an ethylene group, a propylene group, a butylene group, a pentylene group, a hexylene group, a heptylene group, an octylene group, a nonylene group, a decylene group, an undecylene group, a dodecylene group, a tridecylene group, a tetradecylene group, a pentadecylene group, a hexadecylene group, a heptadecylene group, an octadecylene group, a nonadecylene group, and an eicosylene group; more preferably each independently a C₅₋₁₂ alkylene group;
(2): one of B₁ and B₂ is a linking bond, and the other is a C₁₋₂₀ alkylene group.
   1.1.4. R₁, R₂

R₁ and R₂ are each independently

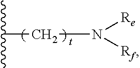

a C₁₋₃₀ aliphatic hydrocarbon group or a residue of C₁₋₃₀ aliphatic hydrocarbon derivative, and at least one of R₁ and R₂ is

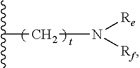

wherein t is an integer from 0 to 12, Rₑ and R_{f} are each independently selected from the group consisting of a C₁₋₁₅ alkyl group, a C₂₋₁₅ alkenyl group, and a C₂₋₁₅ alkynyl group.

In one specific embodiment of the present invention, a C₁₋₃₀ aliphatic hydrocarbon group is preferably a linear alkyl group, a branched alkyl group, a linear alkenyl group, a branched alkenyl group, a linear alkynyl group, or a branched alkynyl group, when said C₁₋₃₀ aliphatic hydrocarbon group is a branched alkyl group, a branched alkenyl group, or a branched alkynyl group, its structure can be represented by

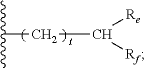

the structure of said residue of C₁₋₃₀ aliphatic hydrocarbon derivative is represented by

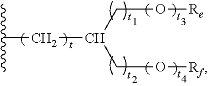

wherein t is an integer from 0 to 12, t₁ and t₂ are each independently an integer from 0 to 5, t₃ and t₄ are each independently 0 or 1, not 0 simultaneously; wherein, Rₑ and R_{f} are each independently a C₁₋₁₅ alkyl group, a C₂₋₁₅ alkenyl group, or a C₂₋₁₅ alkynyl group.

In one specific embodiment of the present invention, a C₁₋₃₀ aliphatic hydrocarbon group or a residue of C₁₋₃₀ aliphatic hydrocarbon derivative is preferably selected from the following structures:

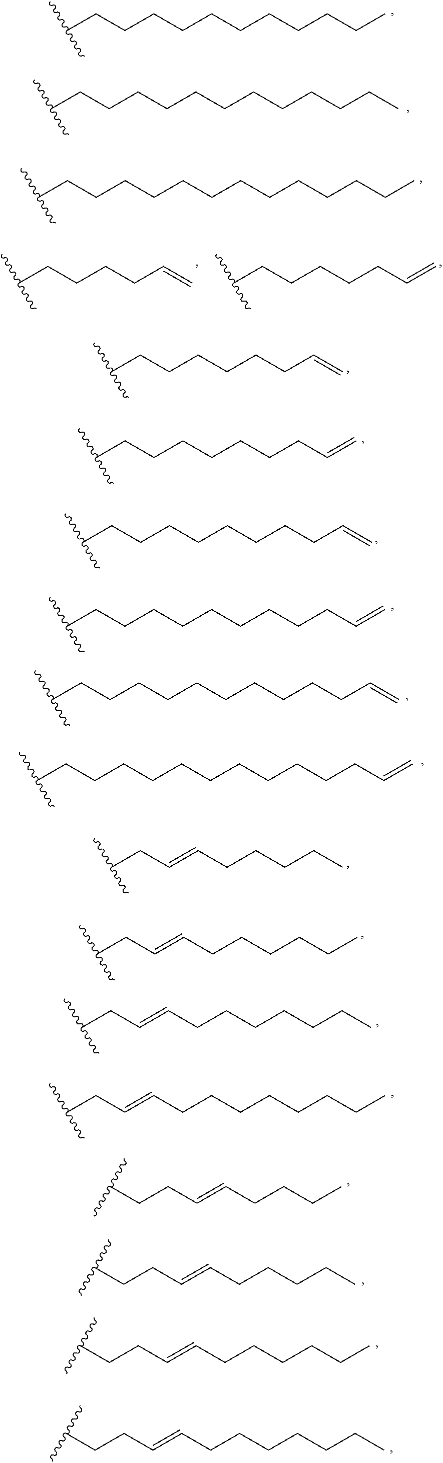

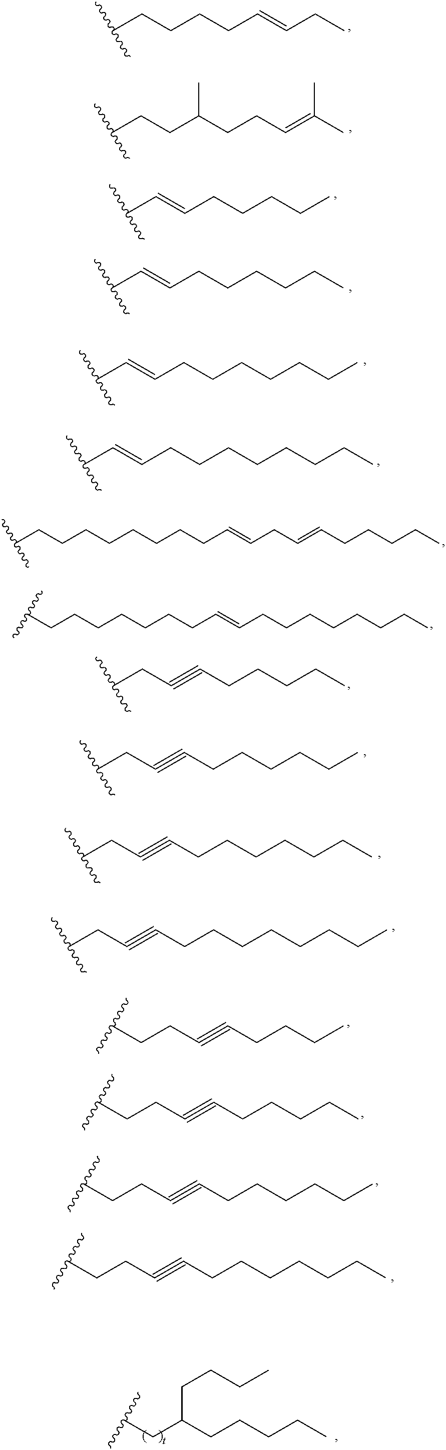

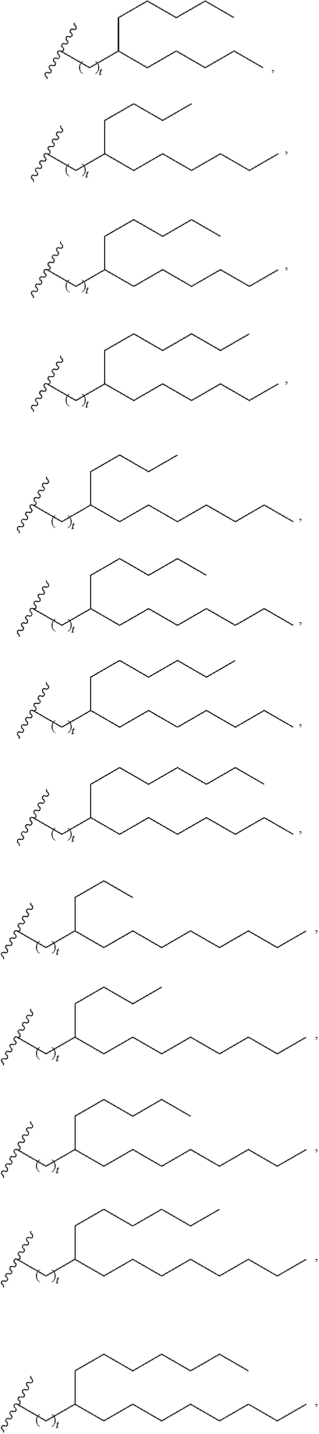

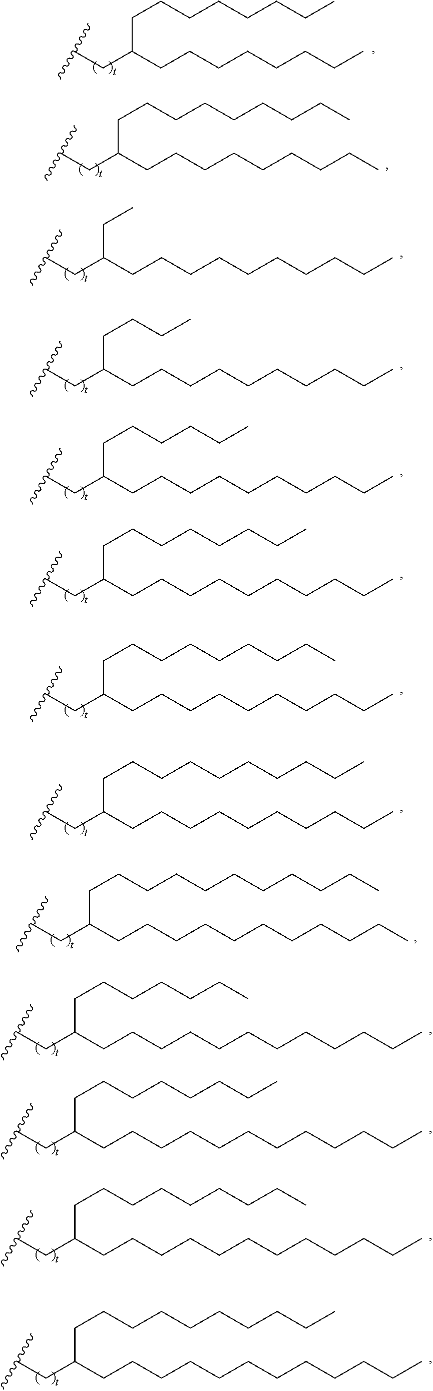

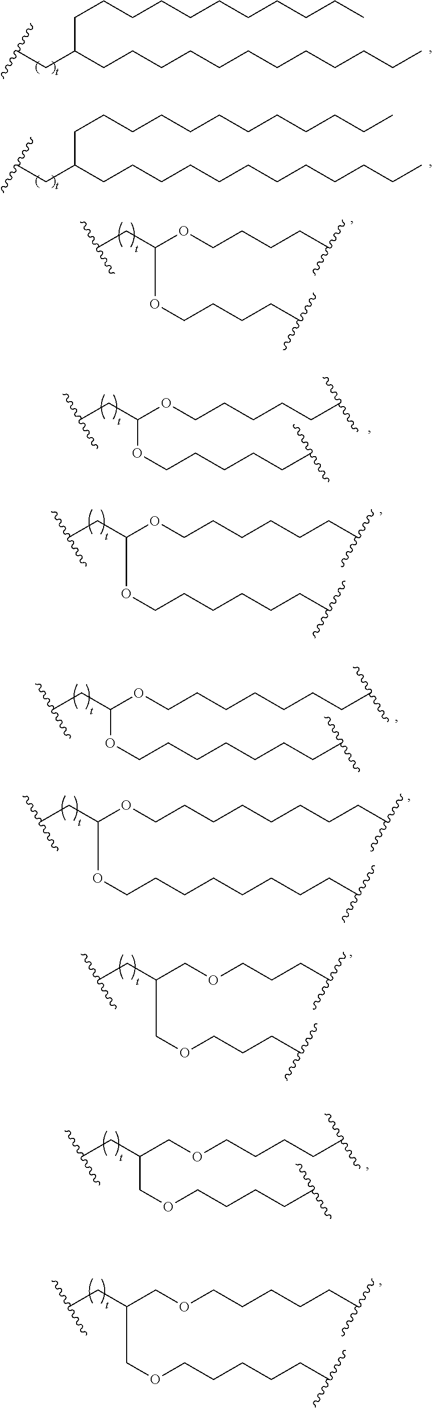

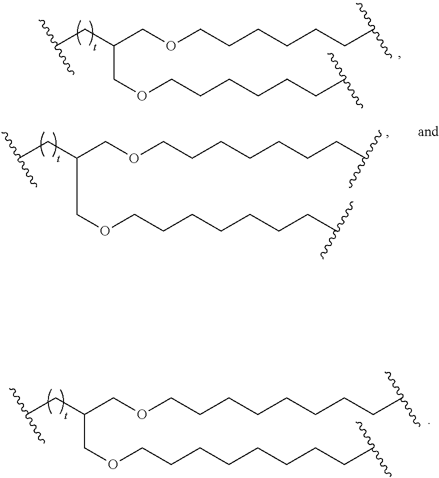

In one specific embodiment of the present invention, Rₑ and R_{f} in said

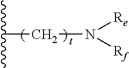

are each independently a C₁₋₁₅ alkyl group, selected from the group consisting of a methyl group, an ethyl group, a propyl group, a butyl group, a pentyl group, a hexyl group, a heptyl group, an octyl group, a nonyl group, and a decyl group; said

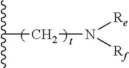

is selected from the following structure:

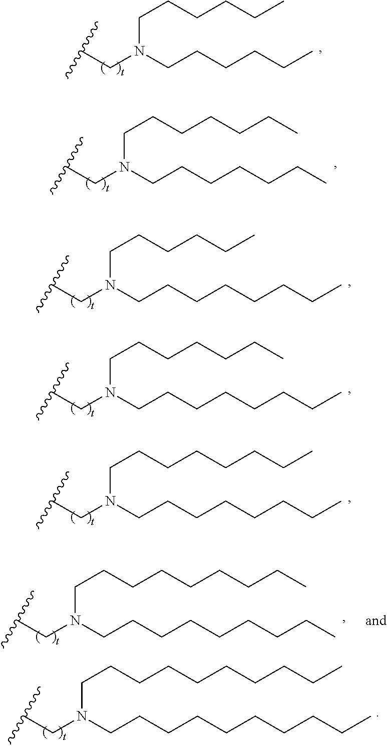

### 1.1.5. R₃

In the present invention, R₃ is a hydrogen atom, —R_{d}, —OR_{d}, —NR_{d}R_{d}, —SR_{d}, —C(═O)R_{d}, —C(═O)OR_{d}, —OC(═O)R_{d}, —OC(═O)OR_{d}, or

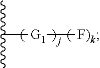

wherein, R_{d} is, at each occurrence, independently a C₁₋₁₂ alkyl group, two R_{d} in NR_{d}R_{d} can be joined to form a ring, G₁ is a terminal branching group with the valence of k+1, j is 0 or 1, F contains functional group R₀₁, when j is 0, G₁ is absent; when j is 1, G₁ protrudes F with the number of k, k is an integer from 2 to 8.

In one specific embodiment of the present invention, R₃ is, at each occurrence, preferably independently a hydrogen atom, R_{d}, OR_{d}, —C(═O)R_{d}—, —C(═O)OR_{d}, —OC(═O)R_{d}, —OC(═O)OR_{d}, or

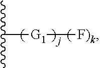

more preferably selected from the group consisting of a hydrogen atom, an alkyl group, an alkoxy group, an alcoholic hydroxyl group, a protected alcoholic hydroxyl group, a thiol group, a protected thiol group, a carboxyl group, a protected carboxyl group, an amino group, a protected amino group, an aldehyde group, a protected aldehyde group, an ester group, a carbonate group, a urethane group, a succinimidyl group, a maleimide group, a protected maleimide group, a dimethylamino group, an alkenyl group, an alkenoate group, an azido group, an alkynyl group, a folate group, a rhodamine group, and a biotinyl group; more preferably selected from the group consisting of H, —(CH₂)ₜOH, —(CH₂)ₜSH, —OCH₃, —OCH₂CH₃, —(CH₂)ₜNH₂, —(CH₂)ₜC(═O)OH, —C(═O)(CH₂)ₜC(═O)OH, —C(═O)CH₃, —(CH₂)ₜN₃, —C(═O)CH₂CH₃, —C(═O)OCH₃, —OC(═O)OCH₃, —C(═O)OCH₂CH₃, —OC(═O)OCH₂CH₃, —(CH₂)ₜN(CH₃)₂, —(CH₂)ₜN(CH₂CH₃)₂, —(CH₂)ₜCHO,

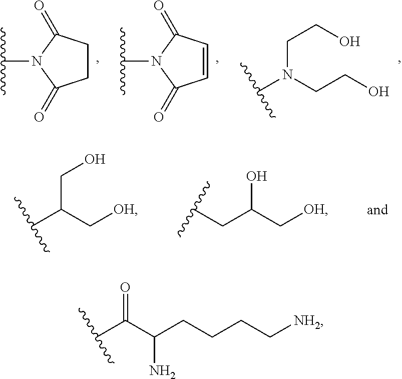

wherein R_{d} is, at each occurrence, independently a C₁₋₁₂ alkyl group.

### 1.1.6. Specific Examples of General Formula

In one specific embodiment of the present invention, when X in the general formula (1) is N, the structure of cationic lipid of the present invention is preferably selected from the group consisting of the following structural formulas:

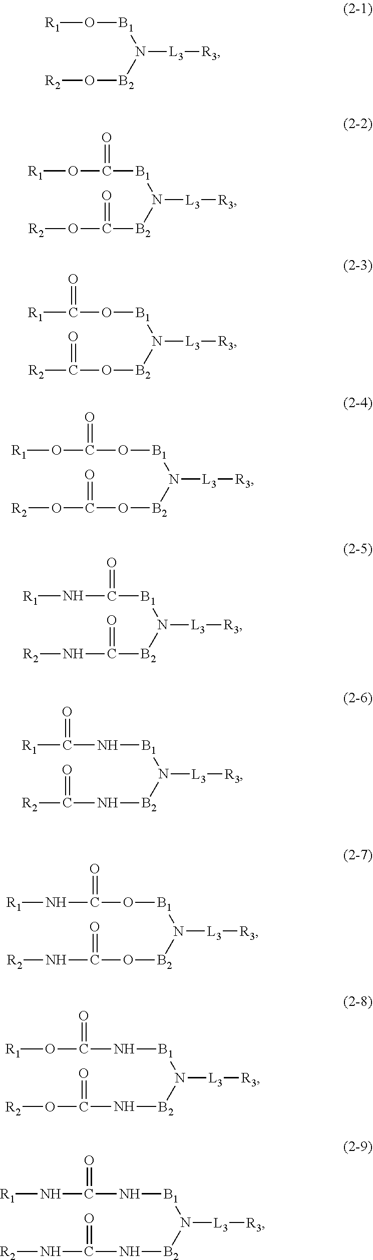

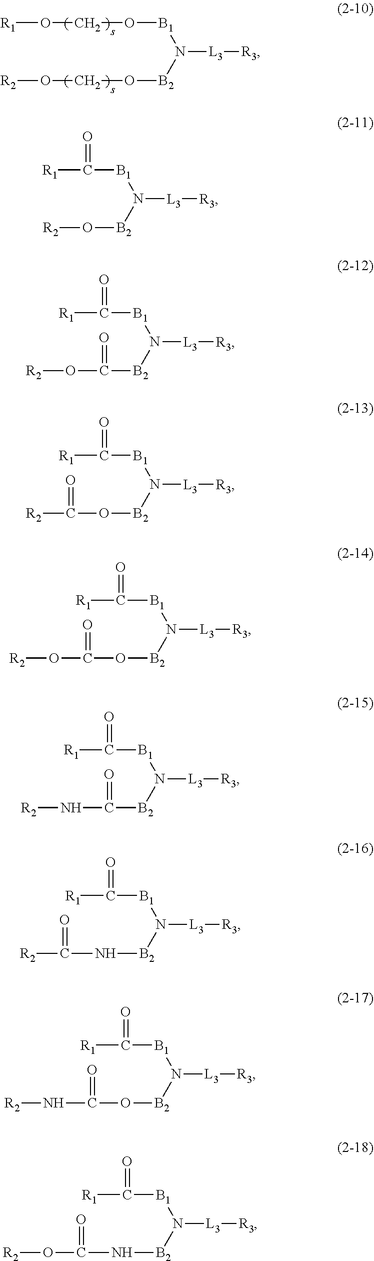

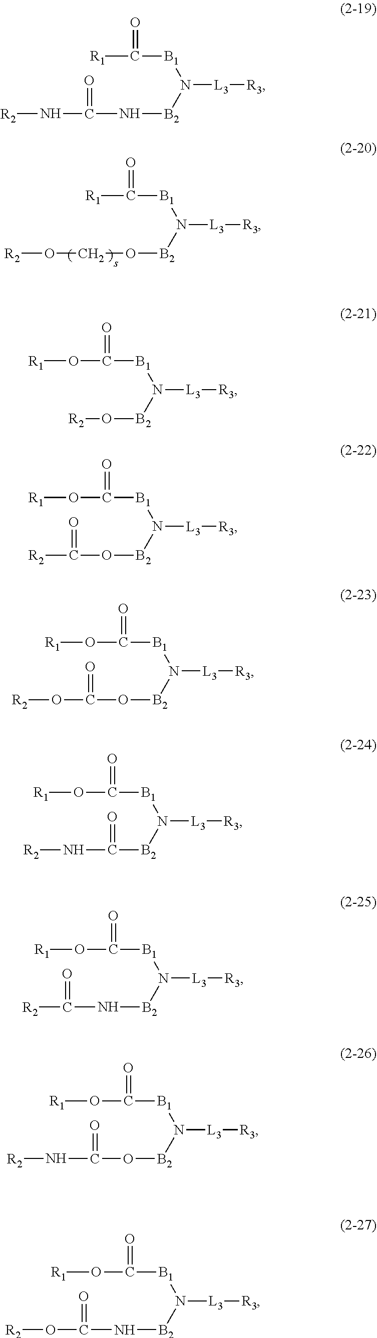

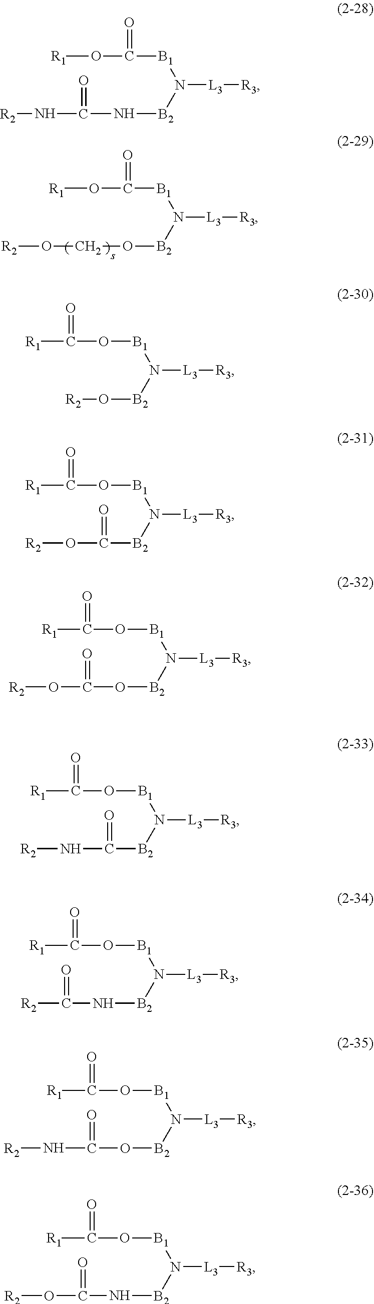

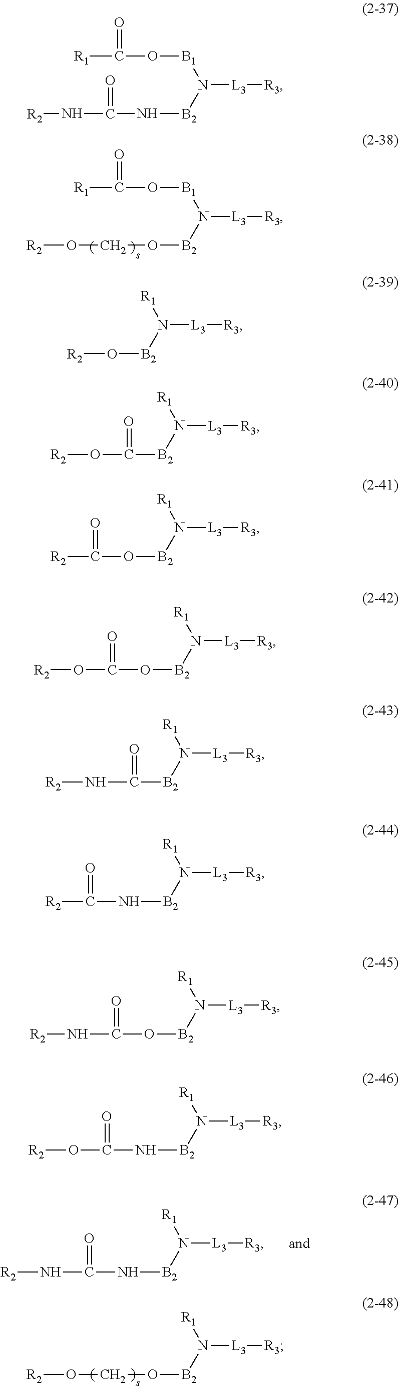

wherein, in the formula (2-39) to formula (2-48), R₁ is, at each occurrence, independently a C₁₋₃₀ aliphatic hydrocarbon group or a residue of C₁₋₃₀ aliphatic hydrocarbon derivative, R₂ is, at each occurrence, independently

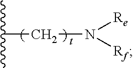

the definitions of s, L₃, B₁, B₂, R₃, R₁, and R₂ are in line with those of the general formula (1), which are not repeated here.

### 1.1.7. Examples of Specific Structures

In some specific embodiments of the present invention, cationic lipids with the following structures are finally obtained, which include but are not limited to the group consisting of the following structures:

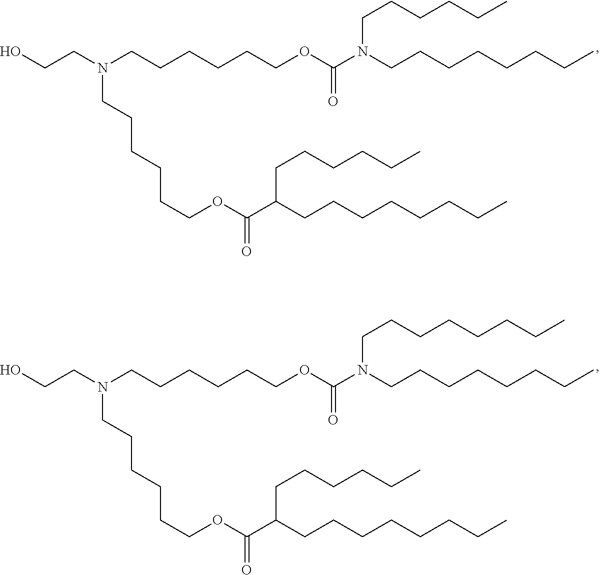 [SMILES: CCCCCCCCC(CCCCCC)C(=O)OCCCCCCN(CCO)CCCCCCOC(=O)N(CCCCCC)CCCCCCCC ; CCCCCCCCC(CCCCCC)C(=O)OCCCCCCN(CCO)CCCCCCOC(=O)N(CCCCCCCC)CCCCCCCC]

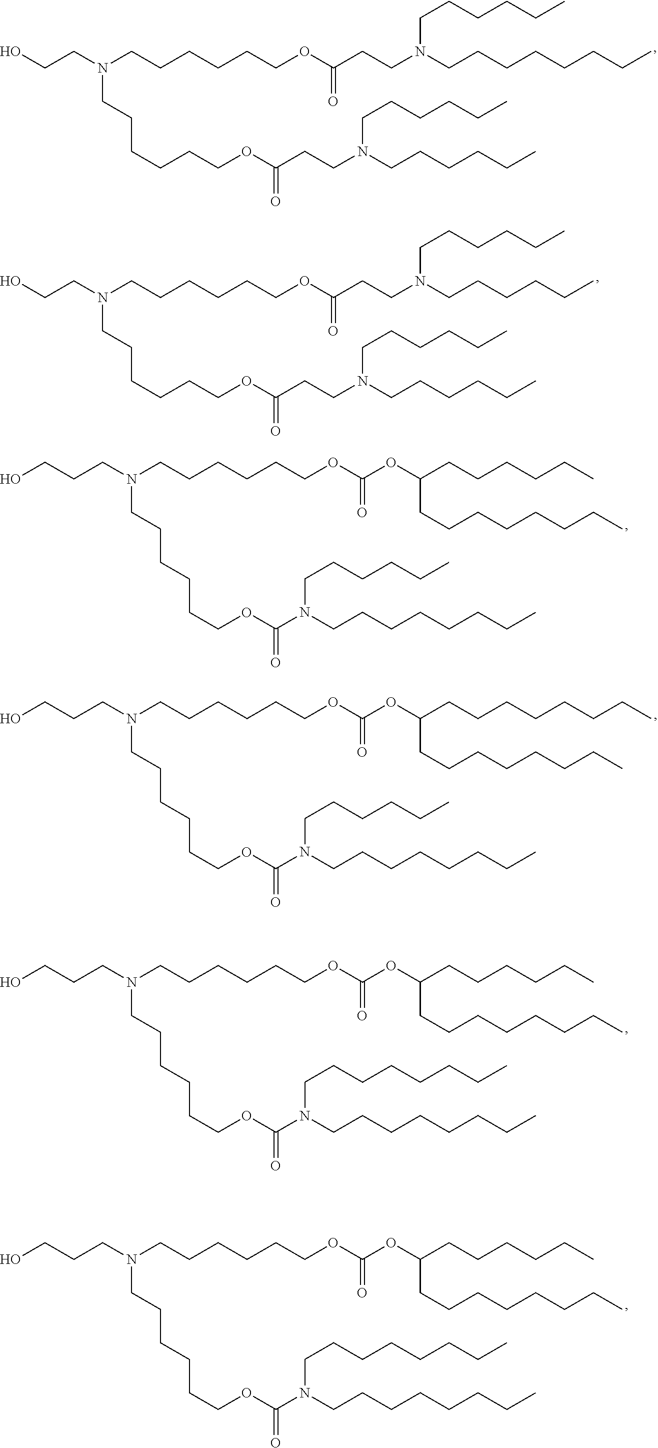

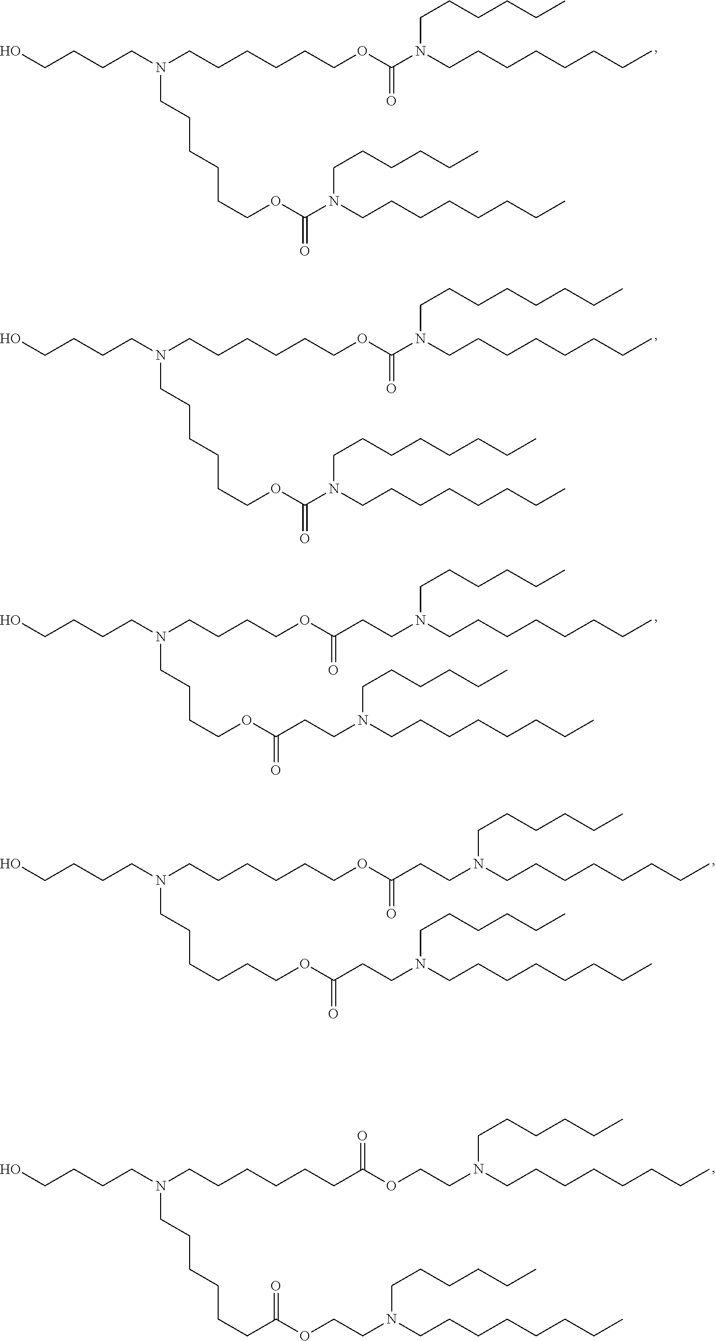 [SMILES: CCCCCCCCN(CCCCCC)C(=O)OCCCCCCN(CCCCO)CCCCCCOC(=O)N(CCCCCC)CCCCCCCC ; CCCCCCCCN(CCCCCCCC)C(=O)OCCCCCCN(CCCCO)CCCCCCOC(=O)N(CCCCCCCC)CCCCCCCC ; CCCCCCCCN(CCCCCC)CCC(=O)OCCCCN(CCCCO)CCCCOC(=O)CCN(CCCCCC)CCCCCCCC ; CCCCCCCCN(CCCCCC)CCC(=O)OCCCCCCN(CCCCO)CCCCCCOC(=O)CCN(CCCCCC)CCCCCCCC ; CCCCCCCCN(CCCCCC)CCOC(=O)CCCCCCN(CCCCO)CCCCCCC(=O)OCCN(CCCCCC)CCCCCCCC]

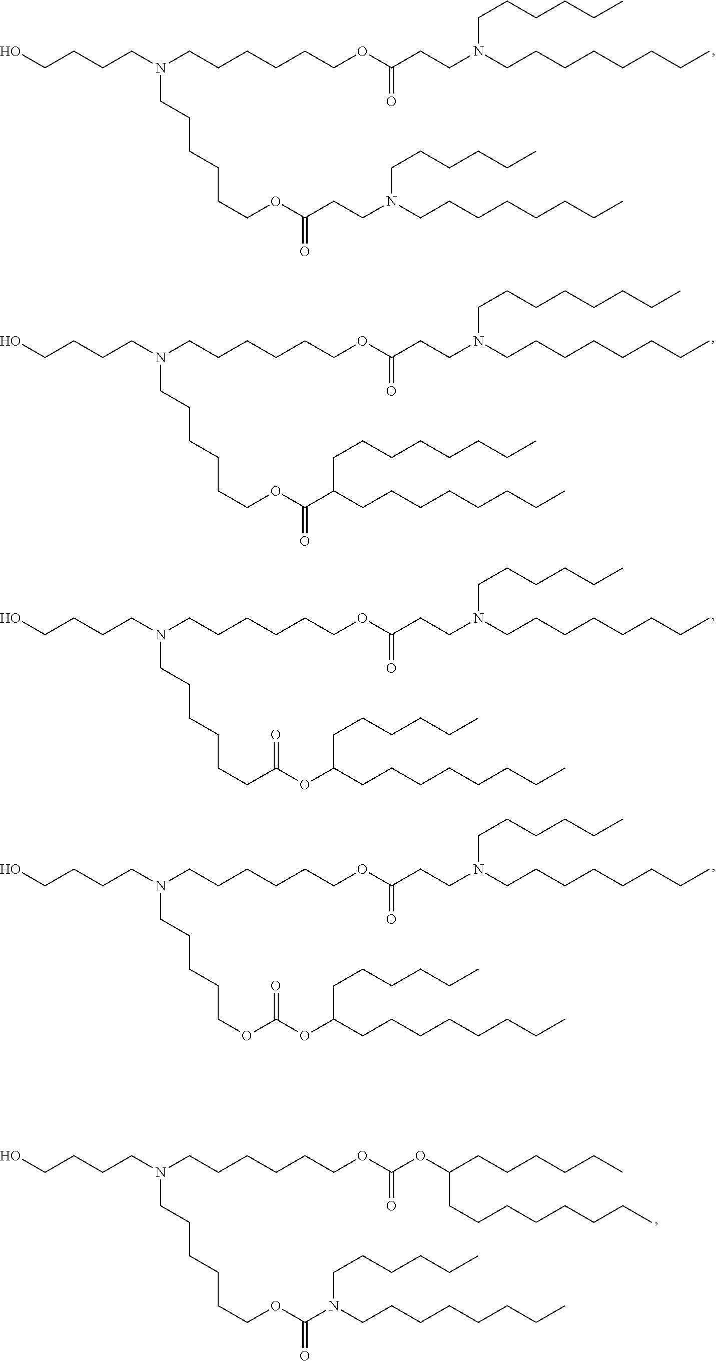

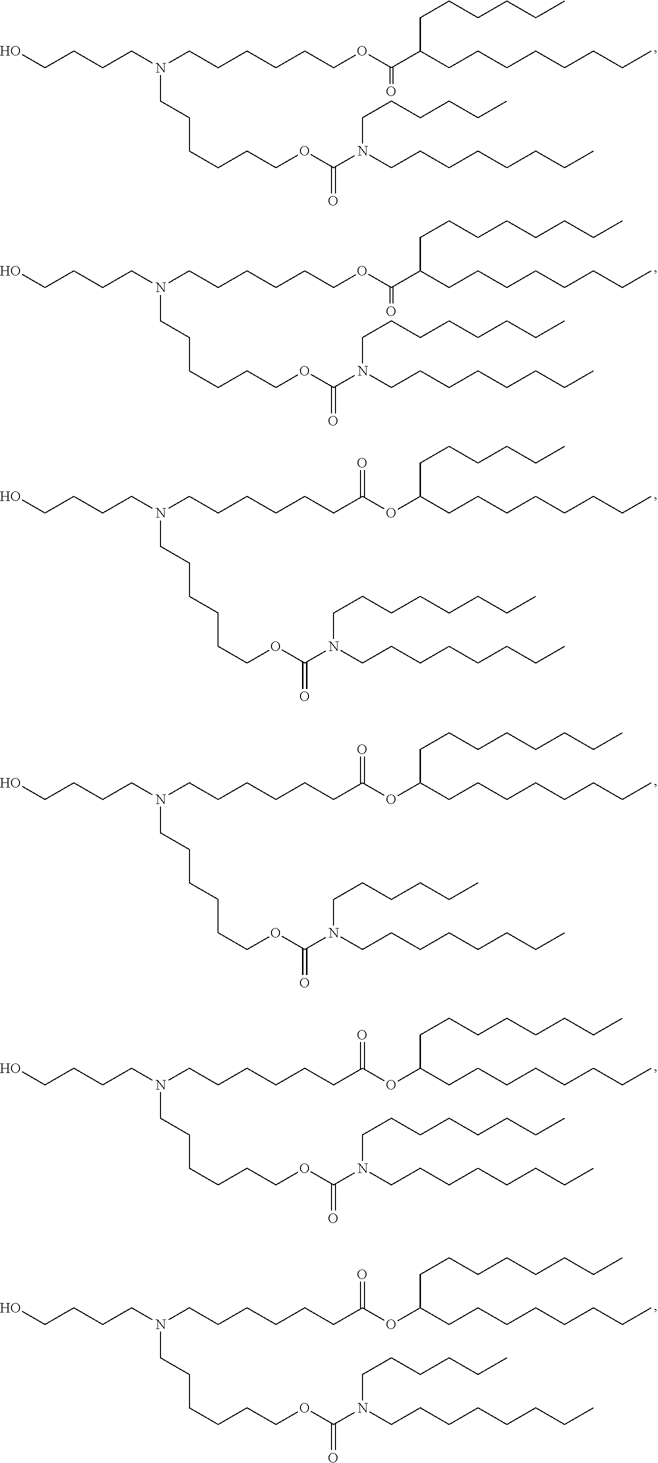 [SMILES: CCCCCCCCC(CCCCCC)C(=O)OCCCCCCN(CCCCO)CCCCCCOC(=O)N(CCCCCC)CCCCCCCC ; CCCCCCCCC(CCCCCCCC)C(=O)OCCCCCCN(CCCCO)CCCCCCOC(=O)N(CCCCCCCC)CCCCCCCC ; CCCCCCCCC(CCCCCC)OC(=O)CCCCCCN(CCCCO)CCCCCCOC(=O)N(CCCCCCCC)CCCCCCCC ; CCCCCCCCC(CCCCCCCC)OC(=O)CCCCCCN(CCCCO)CCCCCCOC(=O)N(CCCCCC)CCCCCCCC ; CCCCCCCCC(CCCCCCCC)OC(=O)CCCCCCN(CCCCO)CCCCCCOC(=O)N(CCCCCCCC)CCCCCCCC ; CCCCCCCCC(CCCCCCCC)OC(=O)CCCCCCN(CCCCO)CCCCCCOC(=O)N(CCCCCC)CCCCCCCC]

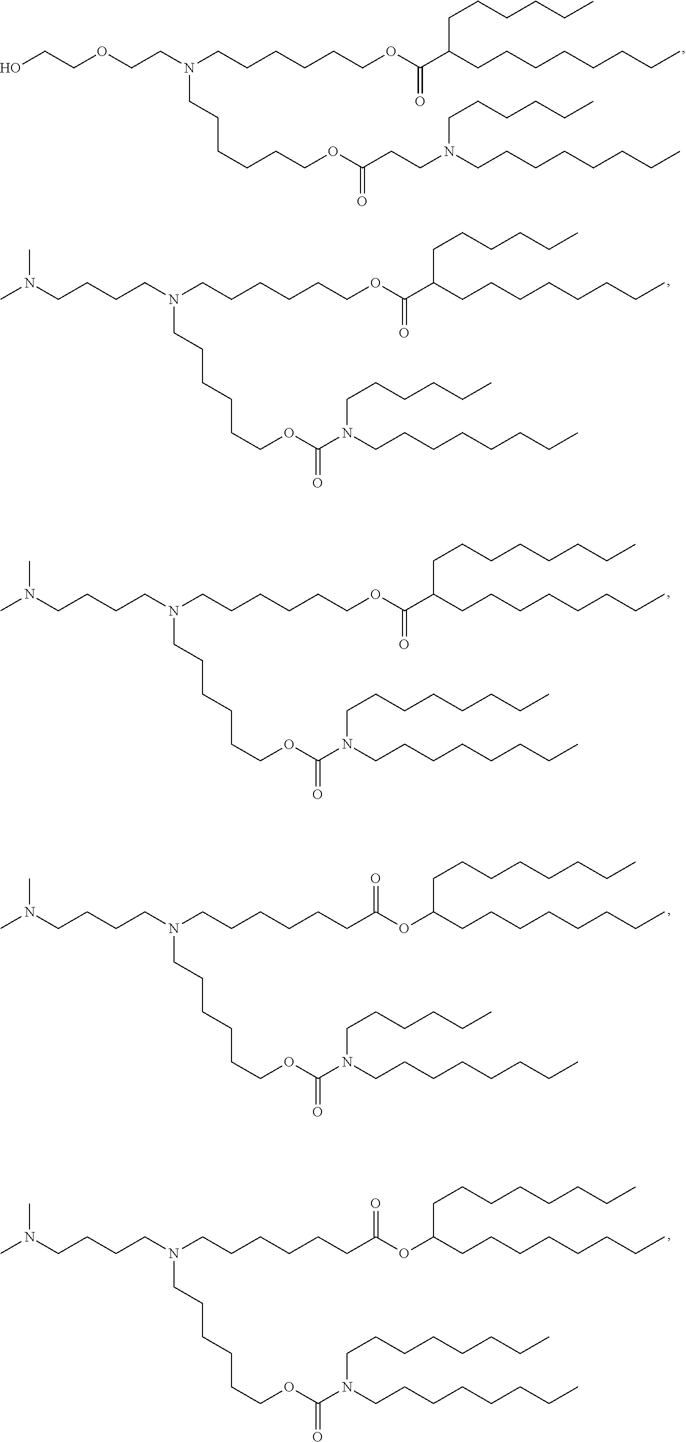

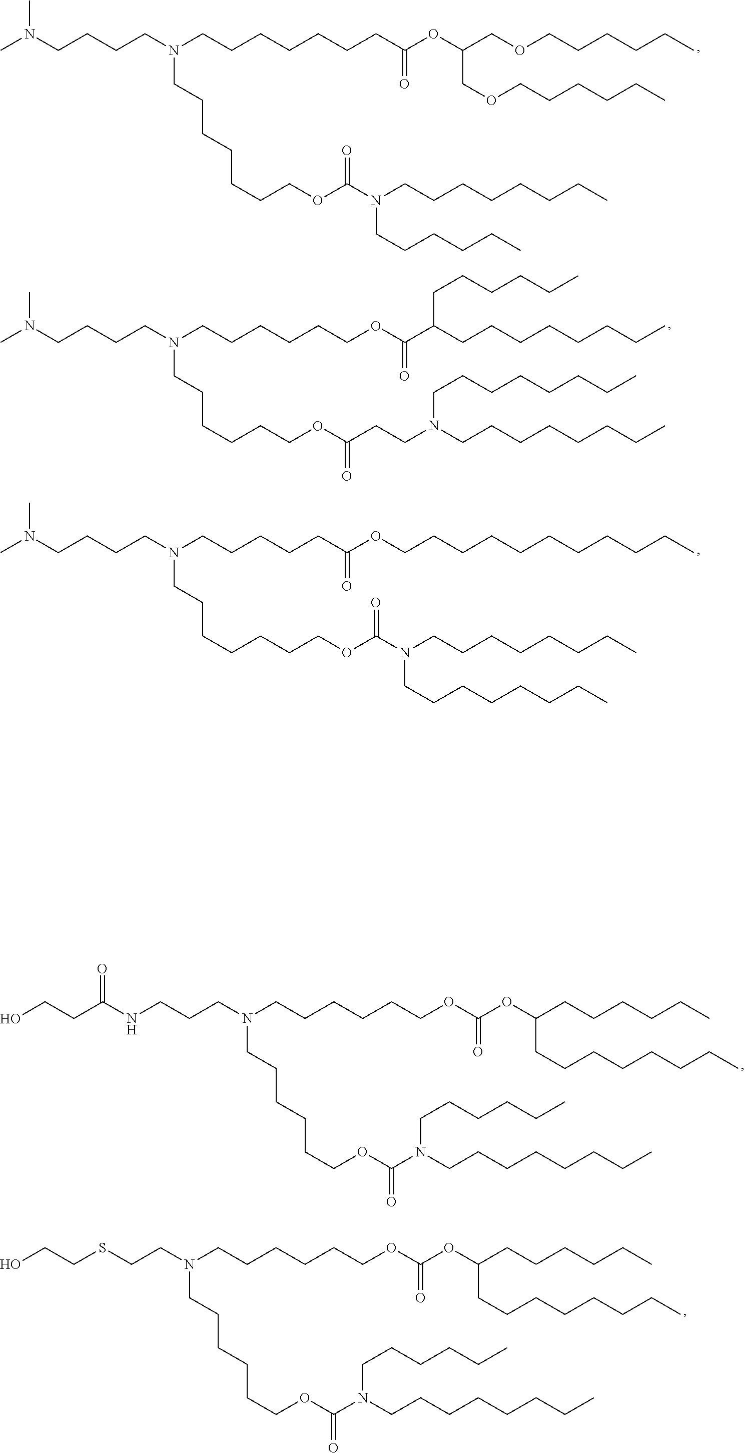

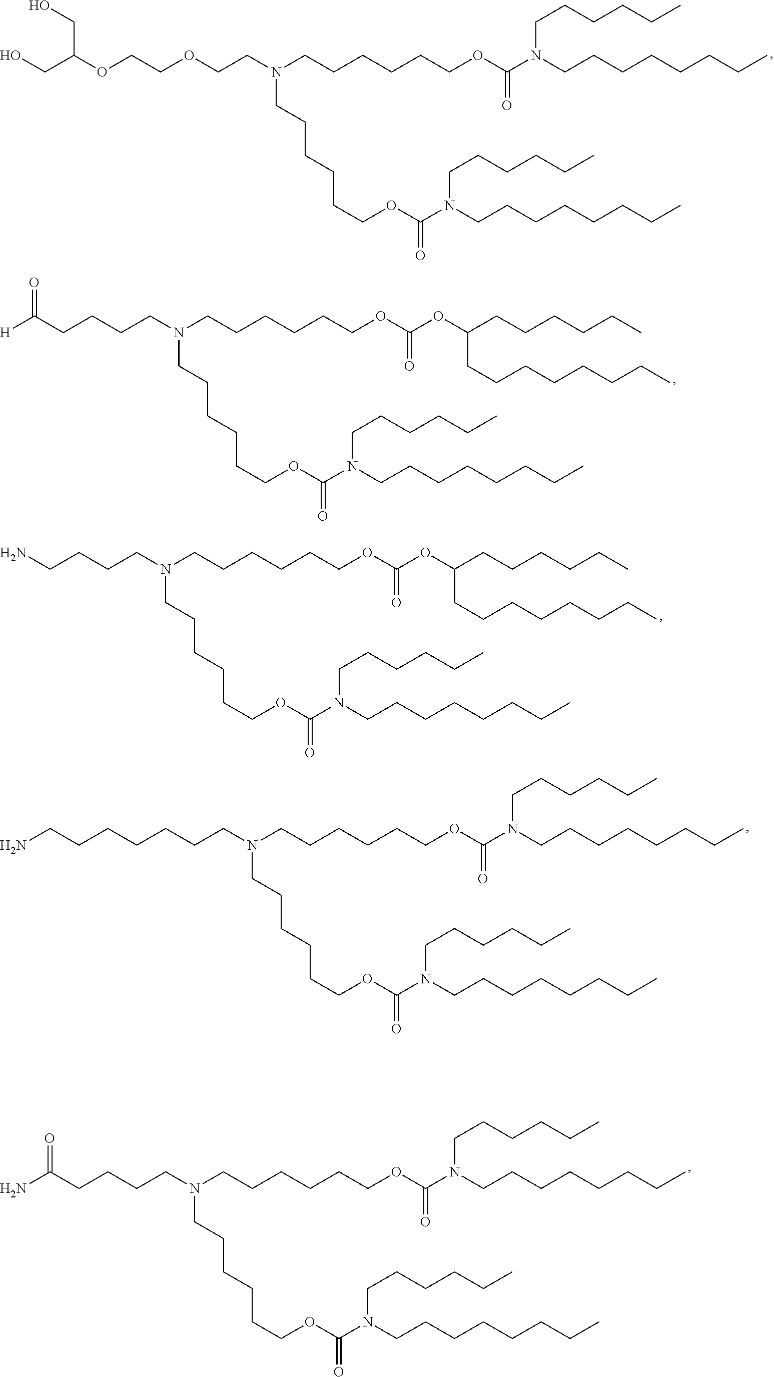

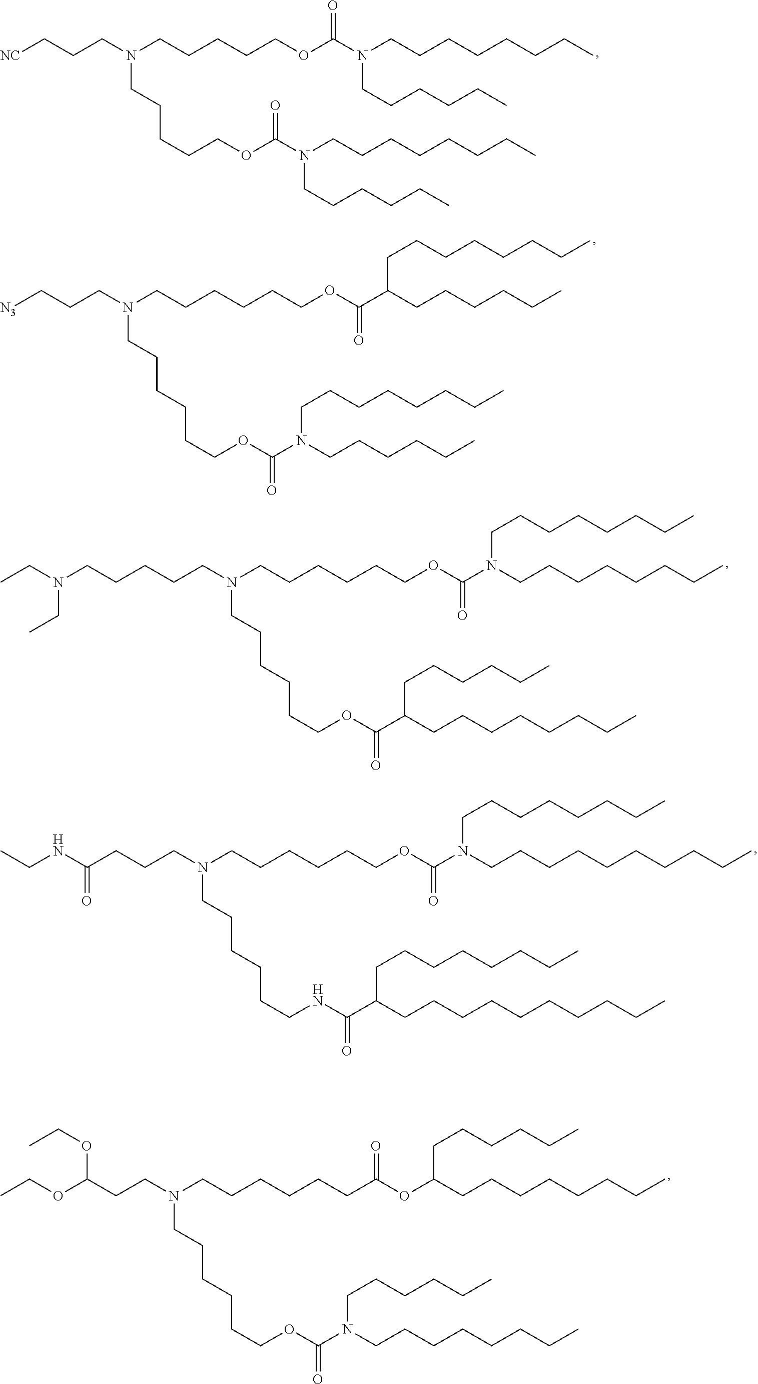

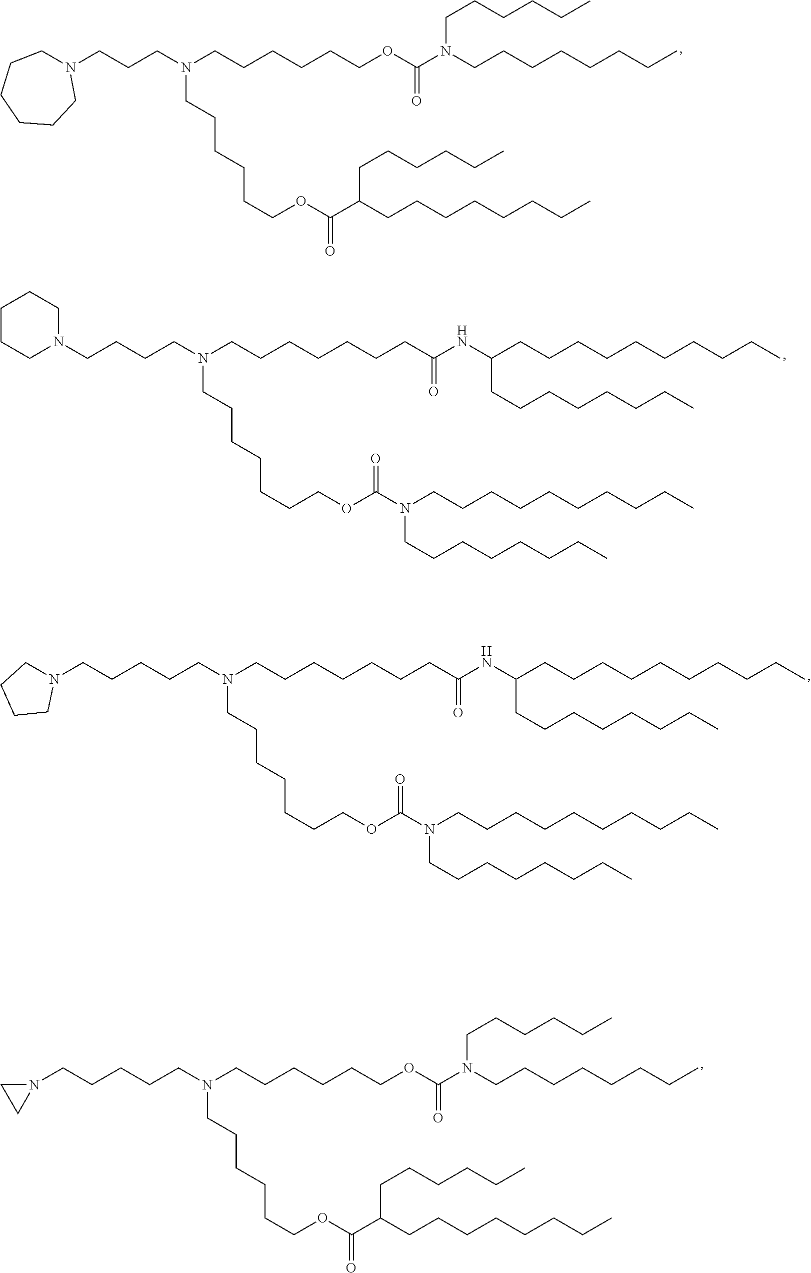 [SMILES: CCCCCCCCC(CCCCCC)C(=O)OCCCCCCN(CCCCCCOC(=O)N(CCCCCC)CCCCCCCC)CCCN1CCCCCC1 ; CCCCCCCCCCC(CCCCCCCC)NC(=O)CCCCCCCN(CCCCCCCOC(=O)N(CCCCCCCC)CCCCCCCCCC)CCCCN1CCCCC1 ; CCCCCCCCCCC(CCCCCCCC)NC(=O)CCCCCCCN(CCCCCCCOC(=O)N(CCCCCCCC)CCCCCCCCCC)CCCCCN1CCCC1 ; CCCCCCCCC(CCCCCC)C(=O)OCCCCCCN(CCCCCCOC(=O)N(CCCCCC)CCCCCCCC)CCCCCN1CC1]

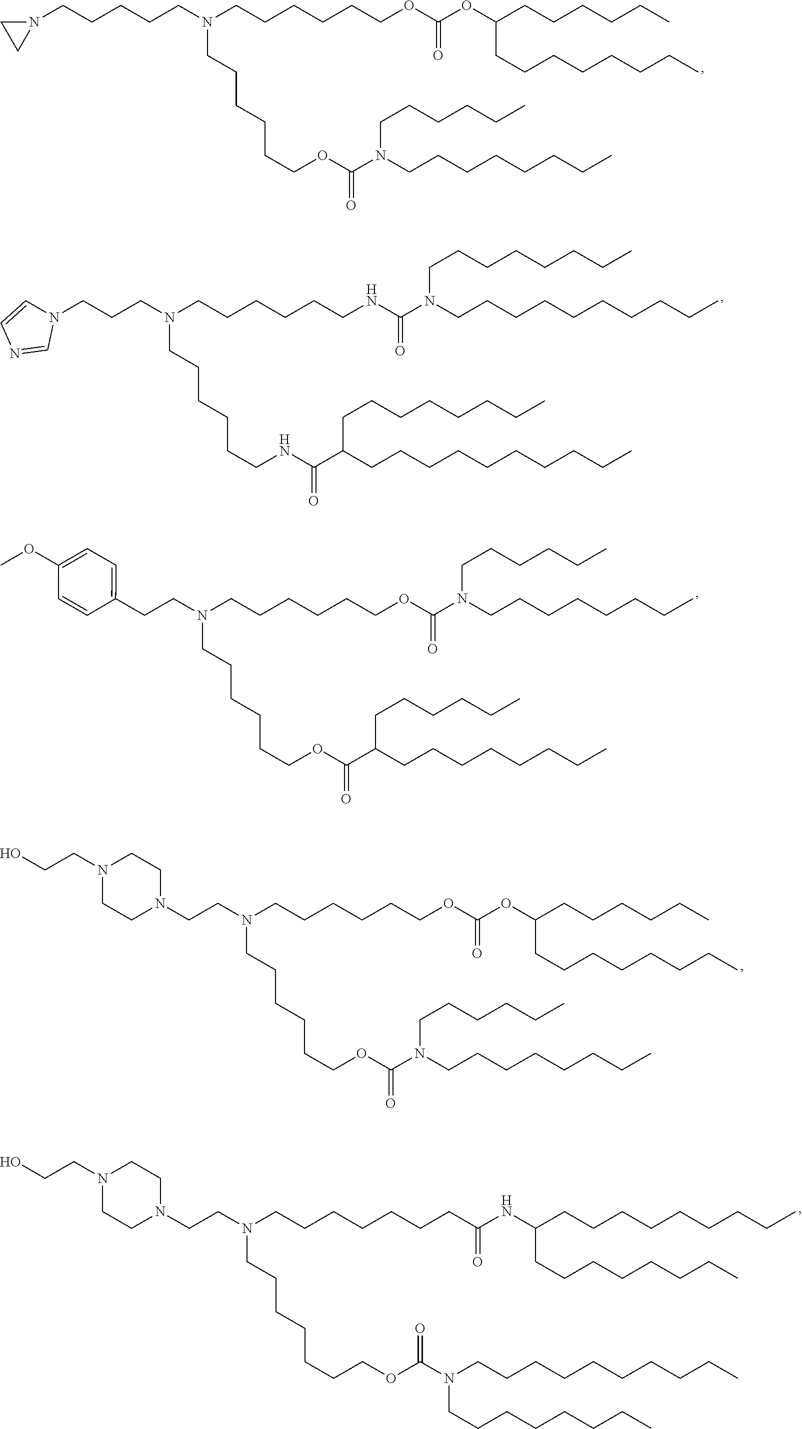

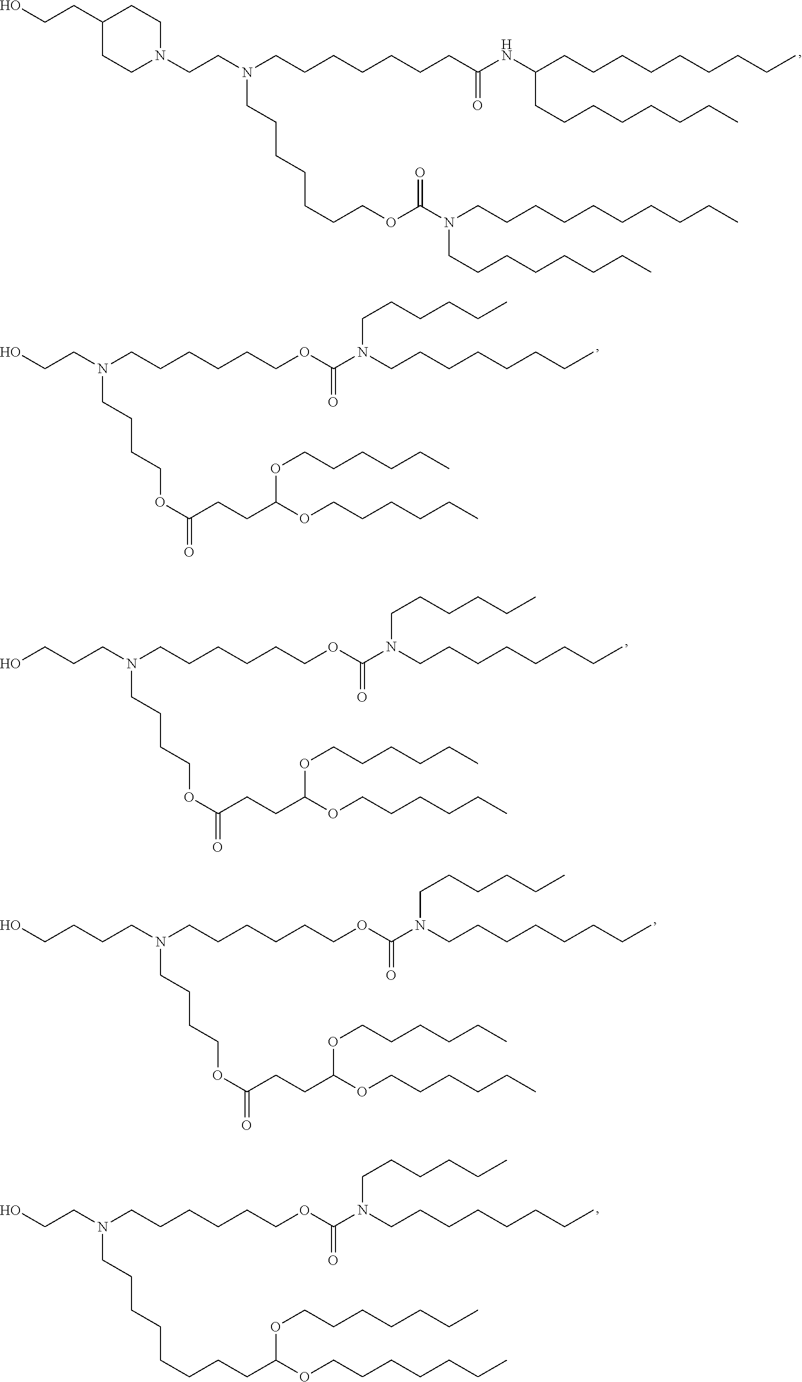

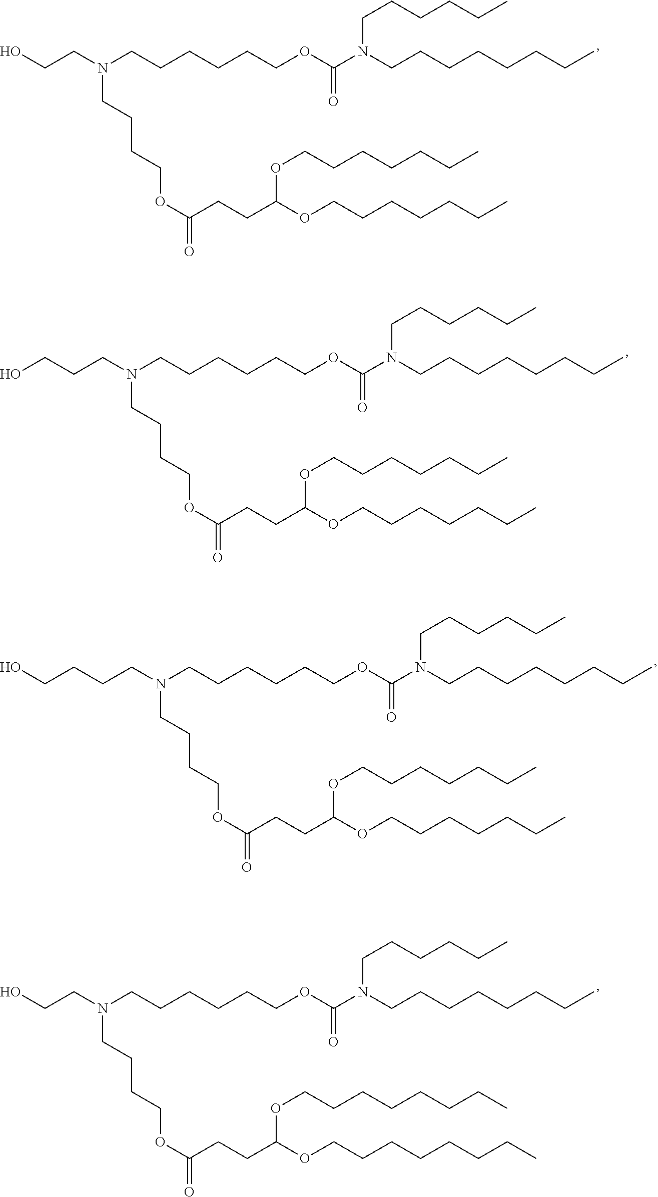 [SMILES: CCCCCCCCN(CCCCCC)C(=O)OCCCCCCN(CCO)CCCCOC(=O)CCC(OCCCCCCC)OCCCCCCC ; CCCCCCCCN(CCCCCC)C(=O)OCCCCCCN(CCCO)CCCCOC(=O)CCC(OCCCCCCC)OCCCCCCC ; CCCCCCCCN(CCCCCC)C(=O)OCCCCCCN(CCCCO)CCCCOC(=O)CCC(OCCCCCCC)OCCCCCCC ; CCCCCCCCOC(CCC(=O)OCCCCN(CCO)CCCCCCOC(=O)N(CCCCCC)CCCCCCCC)OCCCCCCCC]

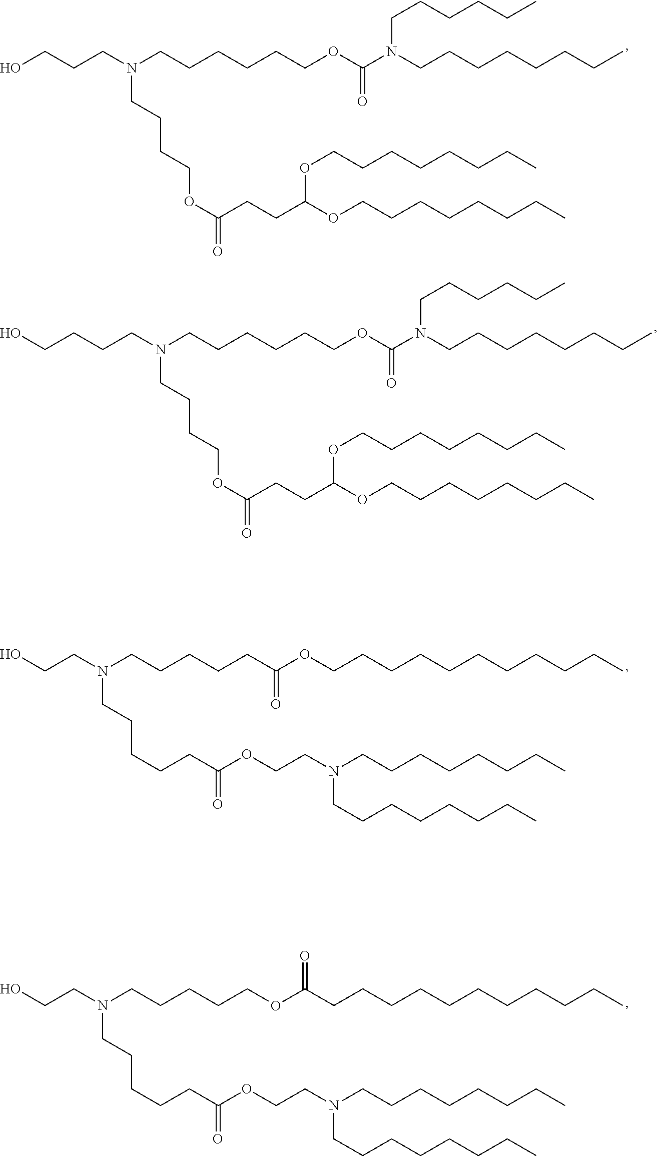 [SMILES: CCCCCCCCOC(CCC(=O)OCCCCN(CCCO)CCCCCCOC(=O)N(CCCCCC)CCCCCCCC)OCCCCCCCC ; CCCCCCCCOC(CCC(=O)OCCCCN(CCCCO)CCCCCCOC(=O)N(CCCCCC)CCCCCCCC)OCCCCCCCC ; CCCCCCCCCCCOC(=O)CCCCCN(CCO)CCCCCC(=O)OCCN(CCCCCCCC)CCCCCCCC ; CCCCCCCCCCCC(=O)OCCCCCN(CCO)CCCCCC(=O)OCCN(CCCCCCCC)CCCCCCCC]

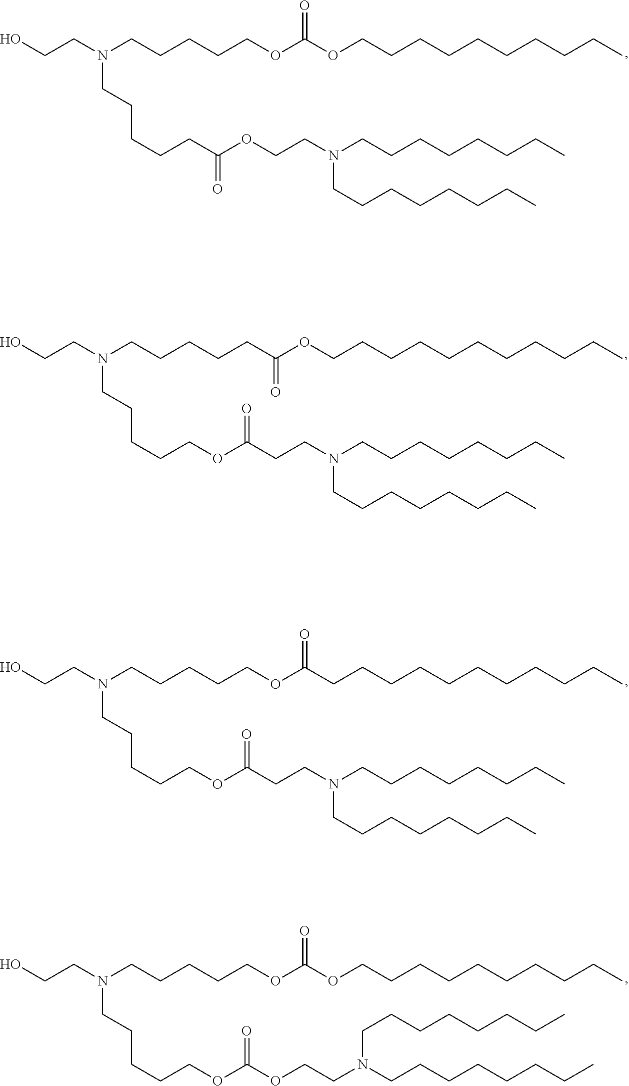 [SMILES: CCCCCCCCCCOC(=O)OCCCCCN(CCO)CCCCCC(=O)OCCN(CCCCCCCC)CCCCCCCC ; CCCCCCCCCCCOC(=O)CCCCCN(CCO)CCCCCOC(=O)CCN(CCCCCCCC)CCCCCCCC ; CCCCCCCCCCCC(=O)OCCCCCN(CCO)CCCCCOC(=O)CCN(CCCCCCCC)CCCCCCCC ; CCCCCCCCCCOC(=O)OCCCCCN(CCO)CCCCCOC(=O)OCCN(CCCCCCCC)CCCCCCCC]

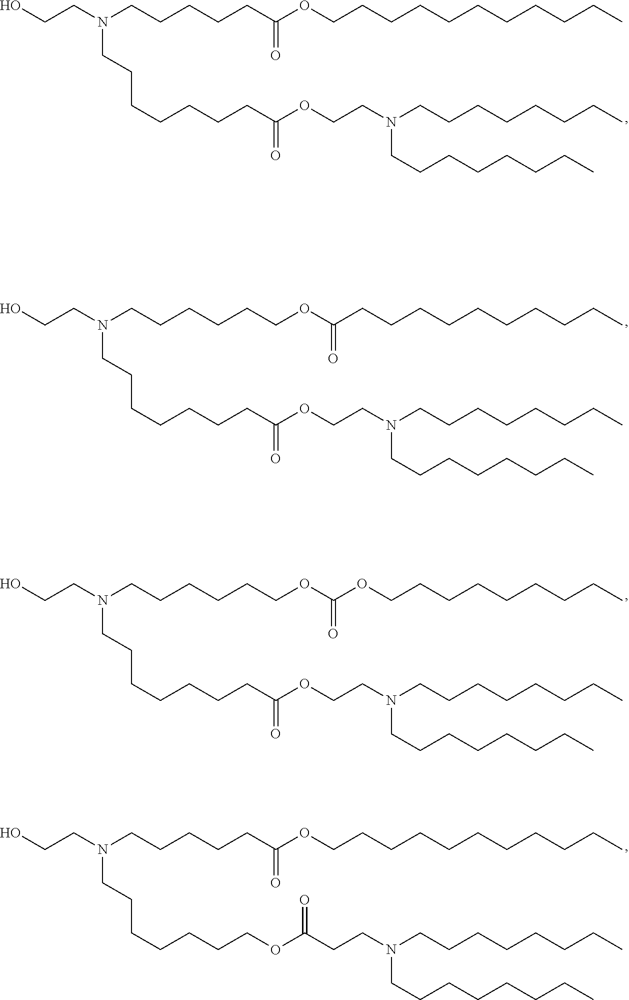 [SMILES: CCCCCCCCCCCOC(=O)CCCCCN(CCO)CCCCCCCC(=O)OCCN(CCCCCCCC)CCCCCCCC ; CCCCCCCCCCC(=O)OCCCCCCN(CCO)CCCCCCCC(=O)OCCN(CCCCCCCC)CCCCCCCC ; CCCCCCCCCOC(=O)OCCCCCCN(CCO)CCCCCCCC(=O)OCCN(CCCCCCCC)CCCCCCCC ; CCCCCCCCCCCOC(=O)CCCCCN(CCO)CCCCCCCOC(=O)CCN(CCCCCCCC)CCCCCCCC]

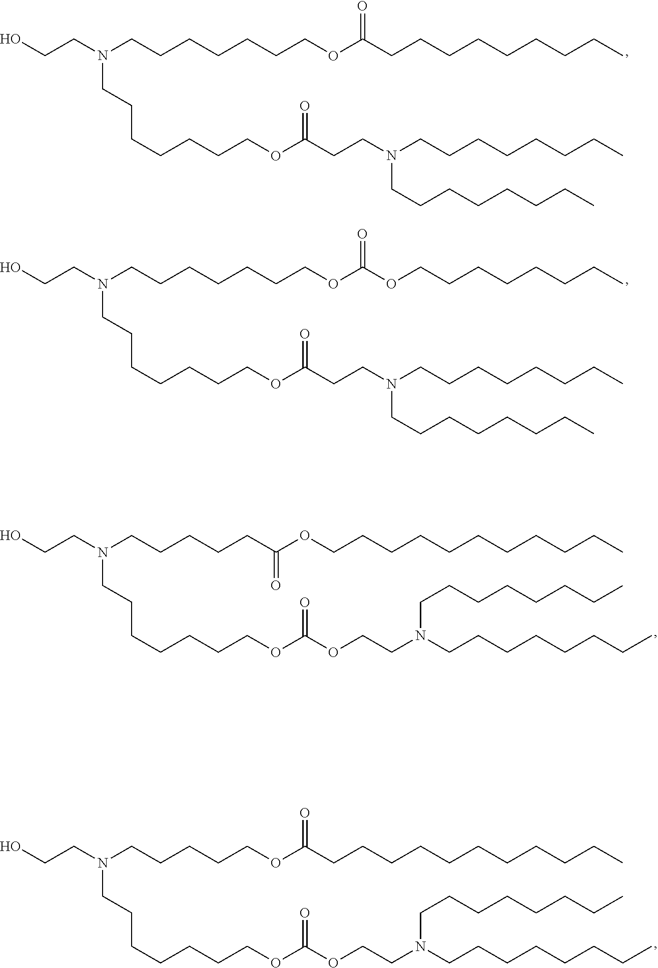 [SMILES: CCCCCCCCCC(=O)OCCCCCCCN(CCO)CCCCCCCOC(=O)CCN(CCCCCCCC)CCCCCCCC ; CCCCCCCCOC(=O)OCCCCCCCN(CCO)CCCCCCCOC(=O)CCN(CCCCCCCC)CCCCCCCC ; CCCCCCCCCCCOC(=O)CCCCCN(CCO)CCCCCCCOC(=O)OCCN(CCCCCCCC)CCCCCCCC ; CCCCCCCCCCCC(=O)OCCCCCN(CCO)CCCCCCCOC(=O)OCCN(CCCCCCCC)CCCCCCCC]

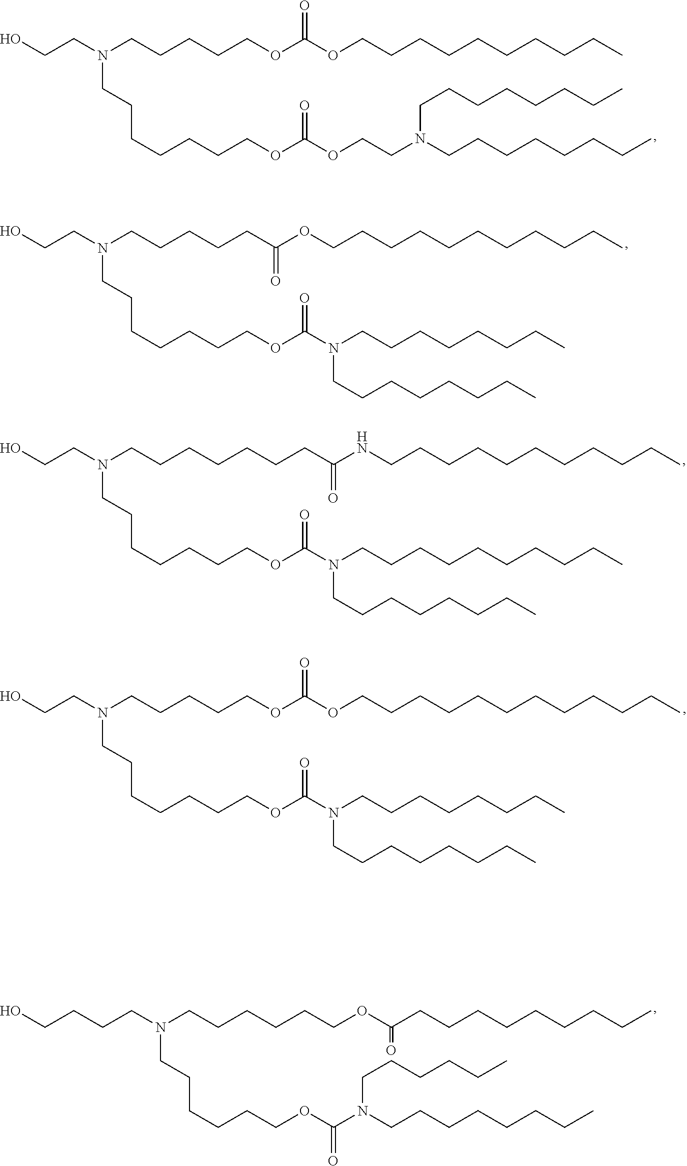 [SMILES: CCCCCCCCCCOC(=O)OCCCCCN(CCO)CCCCCCCOC(=O)OCCN(CCCCCCCC)CCCCCCCC ; CCCCCCCCCCCOC(=O)CCCCCN(CCO)CCCCCCCOC(=O)N(CCCCCCCC)CCCCCCCC ; CCCCCCCCCCCNC(=O)CCCCCCCN(CCO)CCCCCCCOC(=O)N(CCCCCCCC)CCCCCCCCCC ; CCCCCCCCCCCCOC(=O)OCCCCCN(CCO)CCCCCCCOC(=O)N(CCCCCCCC)CCCCCCCC ; CCCCCCCCCC(=O)OCCCCCCN(CCCCO)CCCCCCOC(=O)N(CCCCCC)CCCCCCCC]

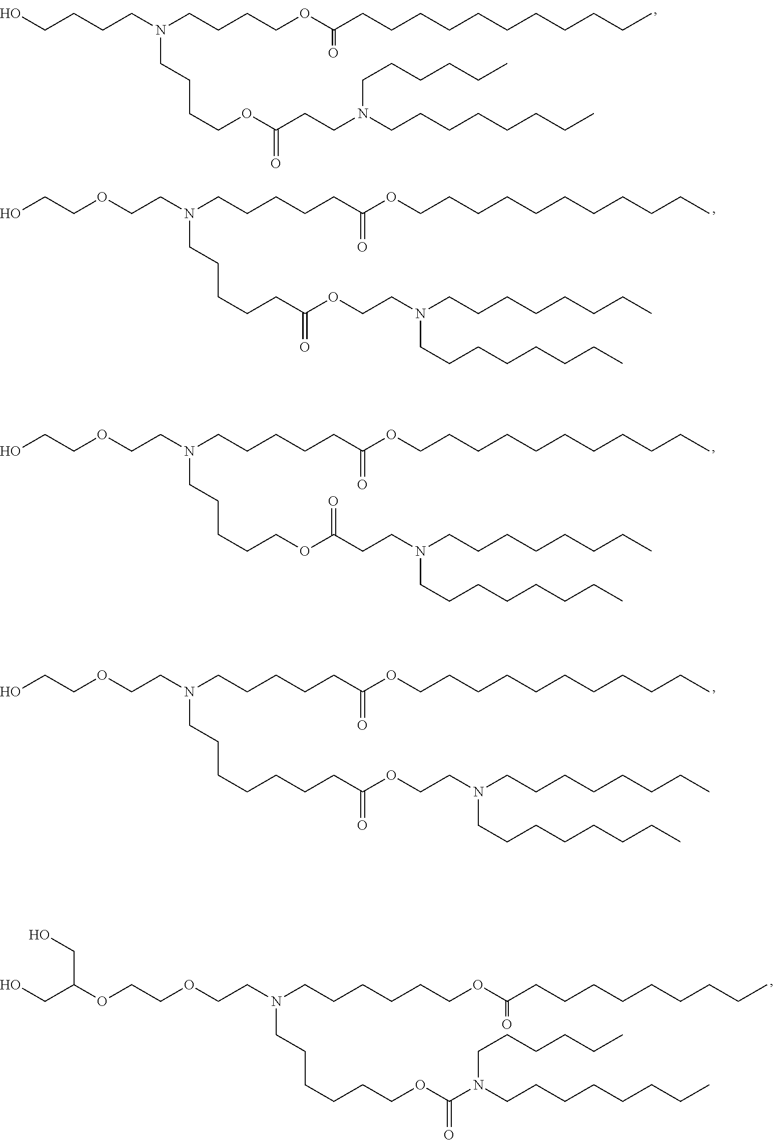

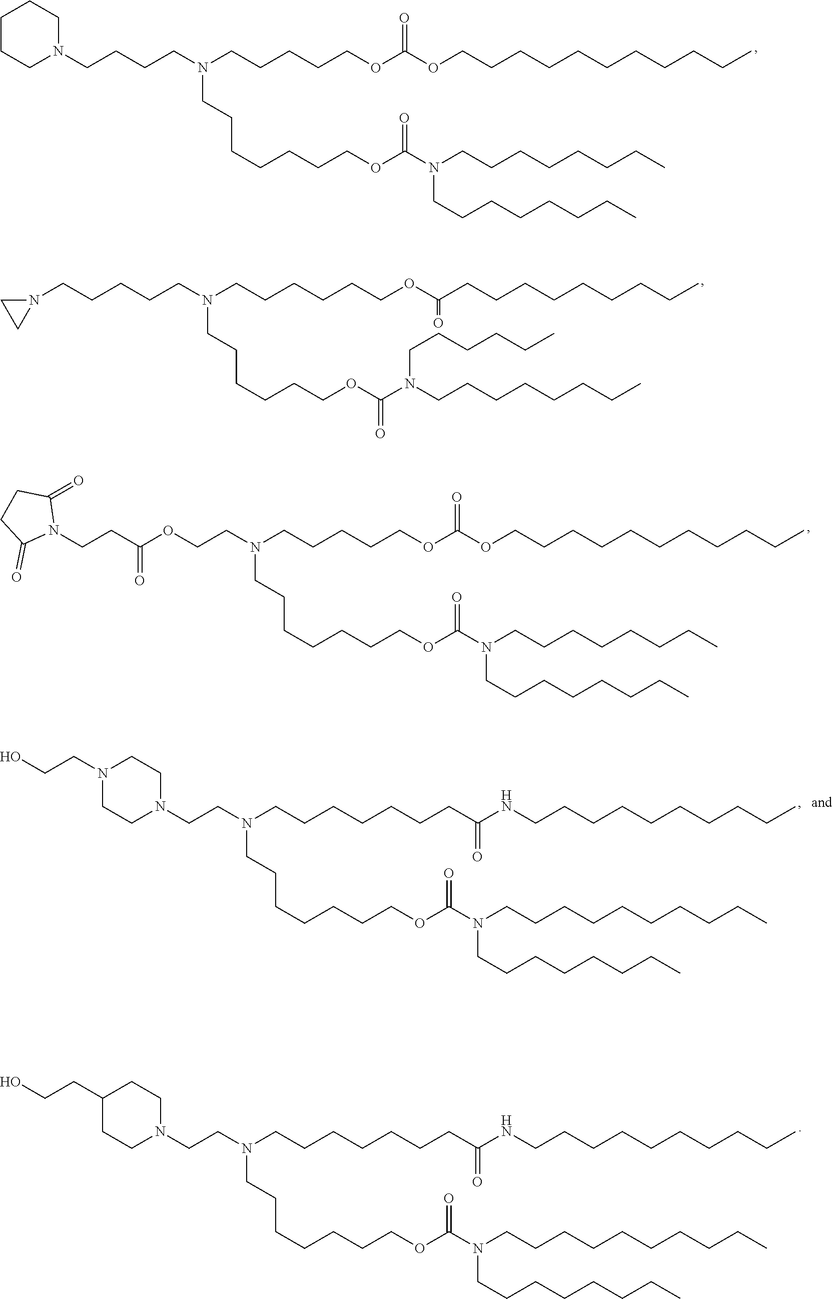 [SMILES: CCCCCCCCCCCOC(=O)OCCCCCN(CCCCCCCOC(=O)N(CCCCCCCC)CCCCCCCC)CCCCN1CCCCC1 ; CCCCCCCCCC(=O)OCCCCCCN(CCCCCCOC(=O)N(CCCCCC)CCCCCCCC)CCCCCN1CC1 ; CCCCCCCCCCCOC(=O)OCCCCCN(CCCCCCCOC(=O)N(CCCCCCCC)CCCCCCCC)CCOC(=O)CCN1C(=O)CCC1=O ; CCCCCCCCCCNC(=O)CCCCCCCN(CCCCCCCOC(=O)N(CCCCCCCC)CCCCCCCCCC)CCN1CCN(CCO)CC1 ; CCCCCCCCCCNC(=O)CCCCCCCN(CCCCCCCOC(=O)N(CCCCCCCC)CCCCCCCCCC)CCN1CCC(CCO)CC1]

### 2. Preparation of Cationic Lipids

In the present invention, any of the above-mentioned cationic lipids can be prepared by methods including but not limited to the following:

### 2.1. Method-1:

Step 1: Carrying out the reaction between small molecule A-1 and small molecule A-2 to obtain the intermediate A-3 containing a divalent linking group L₁, with a reactive group F_{N} at one end and R₁ at the other end; wherein, small molecule A-1 contains a reactive group F₁, small molecule A-2 contains a pair of heterofunctional group F₂ and F_{N}, and F₂ is a reactive group which can react with F₁ to obtain the divalent linking group L₁, F_{N} is the reactive group which can react with amino group or secondary amino group, preferably —OMs, —OTs, —CHO, —F, —Cl or —Br;

Step 2: Carrying out the alkylation reaction between two molecules of the small molecule intermediate A-3 and the primary amine derivative A-4 containing a nitrogen-source terminal group to obtain the cationic lipid A-5, wherein the R₃′ end contains the reactive group R₀₁ or a micro variant form of R₀₁; said micro variant form refers to the group which can be transformed into R₀₁ through any chemical process selected from deprotection, salt complexation and decomplexation, ionization, protonation, deprotonation, and leaving group transformation;
when R₃′ is R₃, the structure of A-5′ is corresponding to the general formula (1);
when R₃′ is not R₃, A-5 is obtained via terminal micro-modification of A-5′ and its structure corresponds to the general formula (1); said terminal micro-modification is selected from the group consisting of deprotection, salt complexation and decomplexation, ionization, protonation, deprotonation, and leaving group transformation, wherein R₁ is the same as R₂, B₁ is the same as B₂, and L₁ is the same as L₂;
wherein, the definitions of Li, L₂, L₃, B₁, B₂, R₃, R₁, and R₂ are in line with those of the general formula (1), and no more repeated here.

The above-mentioned small molecule starting materials A-1, A-2, A-4, and the like, can be obtained by purchase or synthetic means, for example, small molecule A-1 in Example-1.1 is

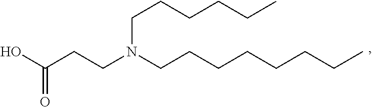 [SMILES: CCCCCCCCN(CCCCCC)CCC(=O)O ; ","]

which can be synthesized with

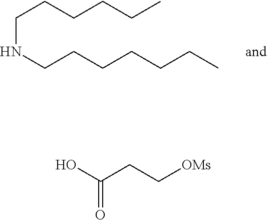

as the starting materials.

### Step 1:

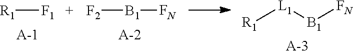

### Step 2:

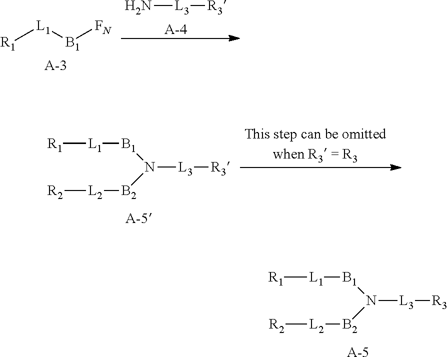

### 2.2 Method-2:

Step 1: Carrying out the reaction between the small molecule B-1 and the small molecule B-2 to obtain the small molecule intermediate B-3 containing a divalent linking group L₁, with a hydroxyl group at one end and R₁ at the other end; wherein, small molecule B-1 contains a reactive group F₁, small molecule B-2 contains a pair of heterofunctional group F₂ and a hydroxyl group (—OH), F₂ is a reactive group which can react with F₁ to obtain divalent linking group L₁;
Step 2: Carrying out the reaction in which the hydroxyl group of the small molecule intermediate B-3 is oxidized to an aldehyde group to obtain the small molecule intermediate B-4 containing an aldehyde group, wherein B₁′ is an alkylene group having one less methylene group than B₁;
Step 3: Carrying out the addition reaction between two molecules of the small molecule intermediate B-4 containing an aldehyde group and the primary amine derivative B-5 containing a nitrogen-source terminal group to obtain the cationic lipid B-6′, wherein the R₃′ end contains the reactive group R₀₁ or a micro variant form of R₀₁; said micro variant form refers to the group which can be transformed into R₀₁ through any chemical process selected from deprotection, salt complexation and decomplexation, ionization, protonation, deprotonation, and leaving group transformation;
when R₃′ is R₃, the structure of B-6′ is corresponding to the general formula (1);
when R₃′ is not R₃, B-6 is obtained via terminal micro-modification of B-6′ and its structure corresponds to the general formula (1); said terminal micro-modification is selected from the group consisting of deprotection, salt complexation and decomplexation, ionization, protonation, deprotonation, and leaving group transformation, wherein R₁ is the same as R₂, B₁ is the same as B₂, and L₁ is the same as L₂;
wherein, the definitions of L₁, L₂, L₃, B₁, B₂, R₃, R₁, and R₂ are in line with those of the general formula (1), and no more repeated here.

The above-mentioned small molecule starting materials B-1, B-2, B-5, and the like, can be obtained by purchase or synthetic means.

### Step 1:

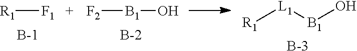

### Step 2:

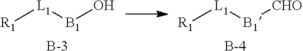

### Step 3:

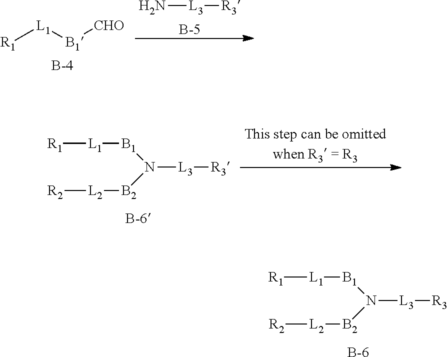

### 2.3. Method-3:

Step 1: Carrying out the reaction between small molecule C-1 and small molecule C-2 to obtain the small molecule intermediate C-3 containing a divalent linking group L₁, with a reactive group F_{N} at one end and R₁ at the other end; and carrying out the reaction between small molecule C-1′ and small molecule C-2′ to obtain the small molecule intermediate C-3′ which contains the divalent linking group L₂, with a reactive group F_{NN} at one end and R₂ at the other end; wherein, small molecule C-1 contains a reactive group F₁, small molecule C-2 contains a pair of heterofunctional group F₂ and F_{N}, F₂ is a functional group which can react with F₁ to obtain the divalent linking group L₁, F_{N} is the reactive group which can react with an amino group or a secondary amino group, preferably —OMs, —OTs, —CHO, —F, —Cl or —Br; small molecule C-1′ contains functional group F₃; small molecule C-2′ contains a pair of heterofunctional group F₄ and F_{NN}, F₄ is a reactive group which can react with F₃ to obtain the divalent linking group L₂; F_{NN} is the functional group which can react with amino group or secondary amino group, preferably —OMs, —OTs, —CHO, —F, —Cl, —Br, —COOH, —COCl, or active carboxyl group, said active carboxyl group refers to the carboxyl group activated with carboxyl activating agents;
Step 2: Carrying out the alkylation reaction between one molecule of the small molecule intermediate C-3 and the primary amine derivative C-4 containing a nitrogen-source terminal group to obtain the secondary amine derivative C-5;
Step 3: Carrying out the reaction between secondary amine derivative C-5 and small molecule intermediate C-3′ to obtain cationic lipid C-6′, wherein the R₃′ end contains the reactive group R₀₁ or a micro variant form of R₀₁; said micro variant form refers to the group which can be transformed into R₀₁ through any chemical process selected from deprotection, salt complexation and decomplexation, ionization, protonation, deprotonation, and leaving group transformation;
when R₃′ is R₃, the structure of C-6′ is corresponding to the general formula (1);
when R₃′ is not R₃, C-6 is obtained via terminal micro-modification of C-6′ and its structure corresponds to the general formula (1); said terminal micro-modification is selected from the group consisting of deprotection, salt complexation and decomplexation, ionization, protonation, deprotonation, and leaving group transformation;
wherein, the definitions of Li, L₂, L₃, B₁, B₂, R₃, R₁, and R₂ are in line with those of the general formula (1), and no more repeated here.

The above-mentioned small molecule starting materials C-1, C-1′, C-2, C-2′, C-4, and the like, can be obtained by purchase or synthetic means, for example, small molecule C-1, namely S6-1, in Example-6 is

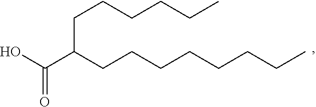 [SMILES: CCCCCCCCC(CCCCCC)C(=O)O ; ","]

which can be obtained by purchase or synthetic means.

### Step 1:

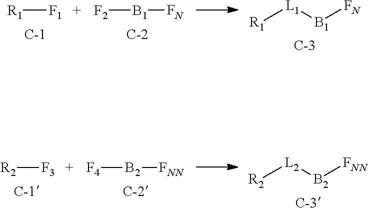

### Step 2:

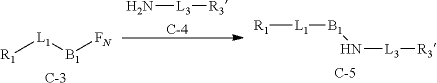

### Step 3:

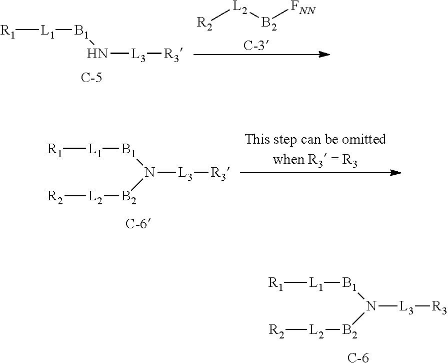

R₁ in the above-mentioned starting material R₁—F₁ can be etherified aliphatic hydrocarbon derivative residue

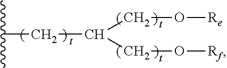

wherein t is, at each occurrence, independently an integer from 0 to 12; Rₑ and R_{f} are each independently a C₁-C₁₅ alkyl group, a C₂-C₁₅ alkenyl group, or a C₂-C₁₅ alkynyl group. More specifically, R₁—F₁ can be

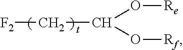

which is obtained by purchase or synthetic means like aldehyde alcohol addition, for example, conducting an addition reaction between a molecule of

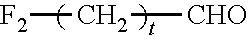

and two molecules of Rₑ—OH to obtain the

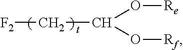

wherein Rₑ is the same as R_{f}; R₁—F₁ can also be

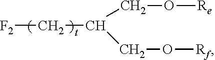

which is obtained by purchase or synthetic means, for example, the compound can be synthetized by the reaction between

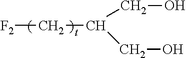

and relevant alkylating agents, said alkylating agent is preferably a halide, for example,

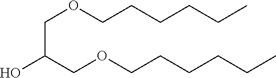 [SMILES: CCCCCCOCC(O)COCCCCCC]

can be obtained by deprotection after the reaction between a molecule of glycerol with a TBS protected hydroxyl group and two molecules of bromohexane.

### 2.4. Method-4:

Carrying out the reaction between trifunctionalized small molecule D-1 containing two identical functional groups F₅ and R₃′ and two molecules of D-2 to obtain the cationic lipid D-3′, wherein small molecule D-2 which contains reactive groups F₆ can react with F₅ to obtain the divalent linking group L₁ or L₂, the R₃′ end contains the reactive group R₀₁ or a micro variant form of R₀₁; said micro variant form refers to the group which can be transformed into R₀₁ through any chemical process selected from deprotection, salt complexation and decomplexation, ionization, protonation, deprotonation, and leaving group transformation;
when R₃′ is R₃, the structure of D-3′ is corresponding to the general formula (1);
when R₃′ is not R₃, D-3 is obtained via terminal micro-modification of D-3′ and its structure corresponds to the general formula (1); said terminal micro-modification is selected from the group consisting of deprotection, salt complexation and decomplexation, ionization, protonation, deprotonation, and leaving group transformation; wherein, R₁ is the same as R₂, and L₁ is the same as L₂;
wherein, the definitions of X, L₁, L₂, L₃, B₁, B₂, R₃, R₁, and R₂ are in line with those of the general formula (1), and no more repeated here.

The above-mentioned small molecule starting materials D-1 and D-2 can be obtained by purchase or synthetic means.

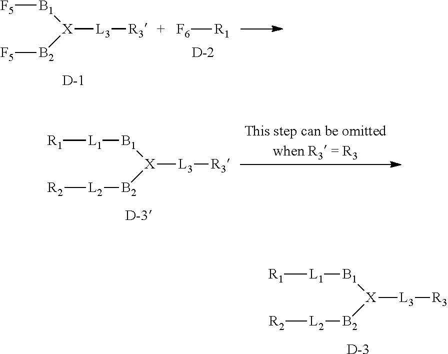

2.5. Description of Relevant Starting Materials and/or Steps in the Preparation Process

### 2.5.1. “Protection” and “Deprotection” of Relevant Groups Involved in the Reaction Process

In the present invention, the reaction process also involves the “protection” and “deprotection” processes of relevant groups. In order to prevent a functional group from affecting the reaction, the functional group is usually protected. In addition, when there are two or more functional groups, only the target functional group reacts selectively, so the other functional groups should be protected. The protecting group not only protects the functional group stably, but also needs to be removed easily as needed. Therefore, in organic synthesis, it is important to remove only the protecting group bonded to the specified functional group under appropriate conditions.

In the present invention, a “carboxyl protecting group” refers to the protecting group which can be transformed into a carboxyl group via hydrolysis or deprotection. A carboxyl protecting group is preferably an alkyl group (e.g., a methyl group, an ethyl group, a tert-butyl group) or an aralkyl group (e.g., a benzyl group), and more preferably a tert-butyl group (tBu), a methyl group (Me), or an ethyl group (Et). In the present invention, a “protected carboxyl group” refers to the group formed via the protection of a carboxyl group with an appropriate carboxyl protecting group, and is preferably a methoxycarbonyl group, an ethoxycarbonyl group, a t-butoxycarbonyl group, or a benzyloxycarbonyl group. Said carboxyl protecting group can be removed through hydrolysis catalyzed by acids or alkalis, or through pyrolysis reactions occasionally; for example, a t-butyl group can be removed under mild acidic conditions, and a benzyl group can be removed by hydrogenolysis. The reagents used for the removal of carboxyl protecting groups are selected from the group consisting of TFA, H₂O, LiOH, NaOH, KOH, MeOH, EtOH, and combinations thereof, preferably the combination of TFA and H₂O, the combination of LiOH and MeOH, or the combination of LiOH and EtOH. A protected carboxyl group can undergo deprotection and then produce the corresponding free acid; said deprotection can be conducted in the presence of an alkali, and said alkali forms pharmaceutically acceptable salts with said free acid obtained from said deprotection.

In the present invention, the “amino protecting group” includes all the groups which can be used as common amino protecting groups, such as an aryl C₁₋₆ alkyl group, a C₁₋₆ alkoxy C₁₋₆ alkyl group, a C₁₋₆ alkoxycarbonyl group, an aryloxycarbonyl group, a C₁₋₆ alkylsulfonyl group, an arylsulfonyl group, a silyl group, etc. An amino protecting group is preferably selected from the group consisting of a t-butoxycarbonyl group (Boc), a p-methoxybenzyloxycarbonyl group (Moz) and a 9-fluorenylmethoxycarbonyl group (Fmoc). The reagent used for removing amino protecting groups is selected from the group consisting of TFA, H₂O, LiOH, MeOH, EtOH and combinations thereof, preferably selected from the combination of TFA and H₂O, the combination of LiOH and MeOH, and the combination of LiOH and EtOH. The reagent used for removing the Boc protecting group is TFA or HC/EA, preferably TFA. The reagent used for removing the Fmoc protecting group is the N,N-dimethylformamide (DMF) solution containing 20% piperidine.

In the present invention, said hydroxyl group protected by a hydroxyl protecting group is not particularly limited, e.g., an alcoholic hydroxyl group, a phenolic hydroxyl group, and the like. Wherein, said amino group protected by an amino protecting group is not particularly limited, selected from the group consisting of a primary amine, a secondary amine, a hydrazine, an amide, and the like. In the present invention, amino groups are not particularly limited, including but not limited to primary amino groups, secondary amino groups, tertiary amino groups, and quaternary ammonium ions.

In the present invention, the deprotection of protected hydroxyl groups is related to the type of hydroxyl protecting group. Said type of hydroxyl protecting group is not particularly limited; for example, benzyl groups, silyl ethers, acetals, or tert-butyl groups can be used to protect terminal hydroxyl groups, and the corresponding deprotection methods include the following:

### A: Removal of Benzyl Groups

The removal of the benzyl groups can be achieved via hydrogenation using a hydrogenating reducing agent and a hydrogen donor. The water content in this reaction system should be less than 1% in order to facilitate the reaction.

The catalyst for hydrogenation reduction is not particularly limited, preferably palladium or nickel. The carrier is not particularly limited, preferably alumina or carbon, more preferably carbon. The amount of palladium is 1 to 100 wt % of that of compounds containing protected hydroxyl groups, preferably 1 to 20 wt %.

The reaction solvent is not particularly limited, as long as both the starting materials and the products can be dissolved, but is preferably methanol, ethanol, ethyl acetate, tetrahydrofuran, or acetic acid, more preferably methanol. The hydrogen donor is not particularly limited but is preferably hydrogen, cyclohexene, 2-propanol, or ammonium formate, etc. The reaction temperature is preferably in the range of 25 to 40° C. The reaction time is not particularly limited, which is negatively correlated with the amount of catalyst, preferably 1 to 5 hours.

### B: Removal of Acetals and Ketals

The compounds used for protecting hydroxyl groups in the forms of acetals or ketals are preferably ethyl vinyl ether, tetrahydropyran, acetone, 2,2-dimethoxypropane, or benzaldehyde, etc. The removal of such acetals or ketals can be realized under an acidic condition wherein the pH of the solution is preferably 0 to 4. The acid used is not particularly limited, preferably acetic acid, phosphoric acid, sulfuric acid, hydrochloric acid, or nitric acid, more preferably hydrochloric acid. The reaction solvent is not particularly limited as long as it allows the starting materials and the products to be dissolved. The solvent is preferably water. The reaction temperature is preferably 0 to 30° C.

### C: Removal of Silyl Ethers

The compounds used to protect the hydroxyl groups in the form of silyl ether include trimethylsilyl ethers, triethylsilyl ethers, tert-butyldimethylsilyl ethers, tert-butyldiphenylsilyl ethers, etc. The removal of such silyl ethers uses compounds containing fluoride ions; wherein, said compounds are preferably selected from the group consisting of tetrabutylammonium fluoride, tetraethylammonium fluoride, hydrofluoric acid, and potassium fluoride, and more preferably selected from tetrabutylammonium fluoride and potassium fluoride. The molar equivalent of the fluorine-containing compound used is 5 to 20 folds of that of the protected hydroxyl group, preferably 8 to 15 folds. When the molar equivalent of the fluorine-containing compound used is less than 5 folds of that of the protected hydroxyl group, the deprotonation might be incomplete. When the molar equivalent of the removal reagent used exceeds 20 folds of that of the protected hydroxyl group, the excess reagents or compounds will bring difficulty in the purification and possibly cause side reactions in subsequent steps. The reaction solvent is not particularly limited, as long as the reactants and the products can be dissolved, but is preferably an aprotic solvent and more preferably tetrahydrofuran or dichloromethane. The reaction temperature is preferably 0 to 30° C., when the temperature is lower than 0° C., the reaction rate is relatively slow, and the protecting group cannot be completely removed.

### D: Removal of Tert-Butyl Groups

The removal of tert-butyl groups is carried out under an acidic condition wherein the pH of the solution is preferably 0 to 4. The acid is not particularly limited, preferably acetic acid, phosphoric acid, sulfuric acid, hydrochloric acid, or nitric acid, more preferably hydrochloric acid. The reaction solvent is not particularly limited as long as the starting materials and the products can be dissolved. The solvent is preferably water. The reaction temperature is preferably 0° C. to 30° C.

In the terminal functionalization method, it is preferred that q=0, q₁=1, and Z₁ is a 1,2-methylene group. When q is not 0, for example, when a linking group such as an amino acid group or a succinyl group is present between A and R₀₁, the prior art capable of generating Z₂ or Z₁ (including but not limited to alkylation, condensation, click reactions, etc.) can be used, and the preparation process can be carried out referring to the following linear functionalization methods.

### 2.5.2. Alkylation Reaction

In the present invention, the alkylation reaction is preferably based on hydroxyl groups, mercapto groups, or amino groups, corresponding to the formation of ether bonds, thioether bonds, secondary amino groups, and tertiary amino groups, respectively. Examples are as follows:

### 2.5.2.1. Alkylation Reaction Between an Alcohol Substrate and a Sulfonate or Halide

An amine intermediate can be obtained via the nucleophilic substitution with a sulfonate or halide on an alcohol substrate under basic conditions. Wherein, the molar equivalent of the sulfonate or halide is 1 to 50 folds of that of the alcohol substrate, preferably 1 to 5 folds. When the molar equivalent of the sulfonate or halide is less than 1-fold of that of the alcohol substrate, the substitution might be incomplete and cause difficulty in the purification process. When the molar equivalent of the sulfonate or halide exceeds 50 folds of that of the alcohol substrate, the excess reagent tends to bring difficulty in the purification process and might be brought into the subsequent step and therefore cause increased side reactions which further increase the difficulty of purification.

The resulting product is a mixture of ether intermediate, excess sulfonate, and excess halide, and can be purified by means such as anion exchange resin, osmosis, ultrafiltration, etc. Wherein, the anion exchange resin is not particularly limited, as long as the target product can undergo ion-exchange and adsorption in the resin, and is preferably the ion exchange resin of tertiary amines or quaternary ammonia salts, with the matrix being dextran, agarose, polyacrylate, polystyrene, or poly(diphenylethylene), etc. The solvents used for osmosis or ultrafiltration are not limited, generally water or an organic solvent. Said organic solvent is not particularly limited, as long as the product can be dissolved within, but is preferably dichloromethane, chloroform, or the like.

The reaction solvent is not limited, preferably an aprotic solvent such as toluene, benzene, xylene, acetonitrile, ethyl acetate, tetrahydrofuran, chloroform, dichloromethane, dimethylsulfoxide, dimethylformamide, and dimethylacetamide, and more preferably dimethylformamide, dichloromethane, dimethylsulfoxide, or tetrahydrofuran.

The base used can be an organic base (e.g., triethylamine, pyridine, 4-dimethylaminopyridine, imidazole, diisopropylethylamine) or an inorganic base (e.g., sodium carbonate, sodium hydroxide, sodium bicarbonate, sodium acetate, potassium carbonate, potassium hydroxide), preferably an organic base, and more preferably triethylamine or pyridine. The molar equivalent of the base used is 1 to 50, preferably 1 to 10, and more preferably 3 to 5 folds of that of the sulfonate or halide.

### 2.5.2.2. Alkylation Reaction Between an Amine Substrate and a Sulfonate or Halide

An amine intermediate can be obtained via the nucleophilic substitution between an amine substrate and a sulfonate or halide under a basic condition. Wherein, the amount of the sulfonate or halide is 1 to 50 folds of that of the amine substrate, preferably 1 to 5 folds. When the amount of the sulfonate or halide is less than 1-fold of that of the amine substrate, the substitution may not sufficiently proceed and the purification tends to be difficult. When the amount of the sulfonate or halide exceeds 50 folds of that of the amine substrate, the excess reagent tends to cause difficulty in the purification process and might be brought to the subsequent steps to result in increased side reactions which further increase the difficulty of purification.

The resulting product is a mixture of amine intermediate, excess sulfonate, and excess halide, the mixture can be purified by purification means such as anion exchange resin, osmosis treatment, ultrafiltration treatment, or the like. Wherein, the anion exchange resin is not particularly limited as long as the target product can undergo ion-exchange and adsorption in the resin, and is preferably the ion exchange resin of tertiary amines or quaternary ammonia salts, with the matrix being dextran, agarose, polyacrylate, polystyrene, or poly(diphenylethylene), etc. The solvents used for osmosis or ultrafiltration are not limited, generally water or an organic solvent. Said organic solvent is not particularly limited, as long as the product can be dissolved within, but is preferably dichloromethane, chloroform, or the like.

The reaction solvent is not limited, preferably an aprotic solvent such as toluene, benzene, xylene, acetonitrile, ethyl acetate, tetrahydrofuran, chloroform, dichloromethane, dimethylsulfoxide, dimethylformamide, and dimethylacetamide, and more preferably dimethylformamide, dichloromethane, dimethyl sulfoxide, or tetrahydrofuran.

The base used can be an organic base (e.g., triethylamine, pyridine, 4-dimethylaminopyridine, imidazole, diisopropylethylamine) or an inorganic base (e.g., sodium carbonate, sodium hydroxide, sodium bicarbonate, sodium acetate, potassium carbonate, potassium hydroxide), preferably an organic base, and more preferably triethylamine or pyridine. The molar equivalent of the base used is 1 to 50, preferably 1 to 10, and more preferably 3 to 5 folds of that of the sulfonate or halide.

### 2.5.2.3. Alkylation Reaction Between an Amine Substrate and an Aldehyde Derivative

The amine substrate reacts with an aldehyde derivative to obtain an imine intermediate, which is followed by obtaining an intermediate using reducing agents. Wherein, the molar equivalent of the aldehyde derivative is 1 to 20, preferably 1 to 2, and more preferably 1 to 1.5 folds of that of the amine substrate. When the molar equivalent of the aldehyde derivative exceeds 20 folds of that of the amine substrate, the excess reagent tends to cause difficulty in the purification process and might be brought into the subsequent step and therefore increase difficulty in the purification. When the molar equivalent of the aldehyde derivative is less than 1-fold of that of the amine substrate, the reaction might be incomplete, causing further difficulty in the purification. Wherein, the resulting product can be obtained after purification by means such as cation exchange resin, osmosis, ultrafiltration, etc. Said cation exchange resin is not particularly limited, as long as it can undergo ion-exchange with quaternary ammonium cations and realize the isolation. The solvents used for osmosis or ultrafiltration treatment are not limited, generally water or an organic solvent. Said organic solvent is not particularly limited, as long as the product can be dissolved within, but is preferably dichloromethane, chloroform, or the like.

The reaction solvent is not limited, preferably an organic solvent such as methanol, ethanol, water, toluene, benzene, xylene, acetonitrile, ethyl acetate, tetrahydrofuran, chloroform, dichloromethane, dimethyl sulfoxide, dimethylformamide, dimethylacetamide, etc., and more preferably water or methanol.

The reducing agent is not particularly limited, as long as the imine can be reduced to an amine, but is preferably sodium borohydride, lithium aluminum hydride, sodium cyanoborohydride, or Zn/AcOH, etc., and more preferably sodium cyanoborohydride. The amount of the reducing agent used is generally 0.5 to 50 folds and preferably 1 to 10 folds of that of the aldehyde derivative.

### 2.5.3. Trifunctionalized Small Molecule D-1

Trifunctionalized small molecule D-1 contains two identical reactive groups Fₛ and R₃′, wherein Fₛ is a reactive group, the R₃′ end contains the functional group R₀₁ or a micro variant form of R₀₁; said micro variant form refers to the group which can be transformed into R₀₁ through any chemical process selected from deprotection, salt complexation and decomplexation, ionization, protonation, deprotonation, and leaving group transformation.

Specifically, the structure of said trifunctionalized small molecule D-1 includes but is not limited to the following structures:

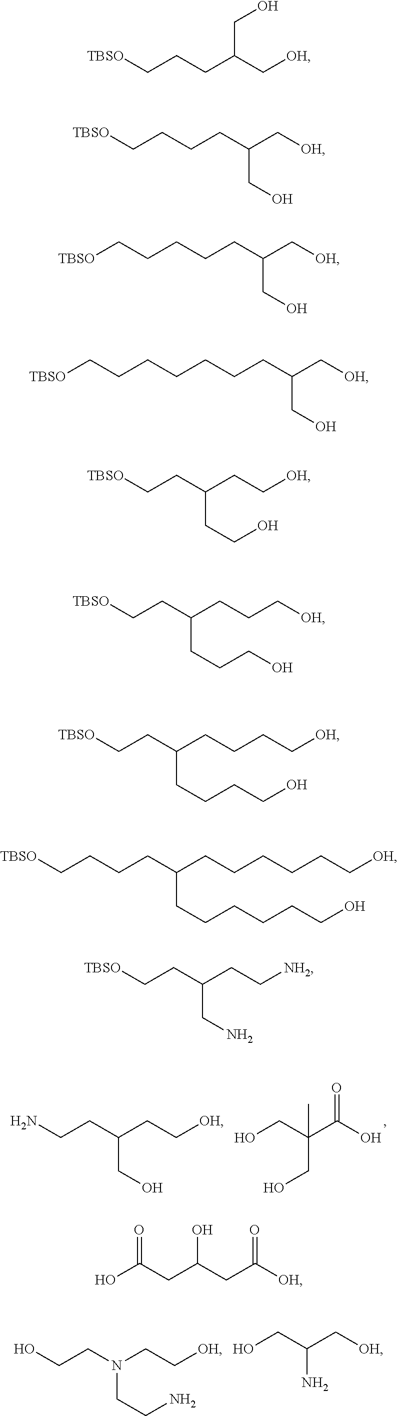

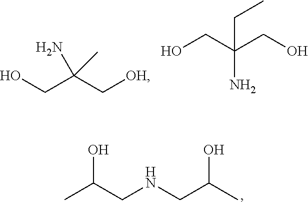

and the like, it also includes the aforementioned trifunctionalized small molecules with relevant groups protected, for example,

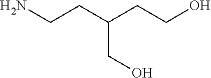

and its amimo group protected form, e.g.,

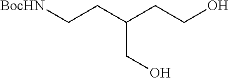 [SMILES: CC(C)(C)OC(=O)NCCC(CO)CCO]

are both included.

### 2.5.4. Linear Functionalization of the Terminal of Chains

The method for linear functionalization of the terminal of chains is not particularly limited, but is relevant to the type of the final functional group or its protected form.

The method for linear functionalization of the terminal hydroxyl group, which starts from the terminal hydroxyl group of compound A-5′ and obtains other functional groups or their protected form -L₃-R₃ through functionalization, the specific preparation method is described in the paragraphs from [0960] to [1205] of the document CN104530417A.

In the present invention, starting materials used in every preparation method can be obtained by purchase or synthetic means.

All the intermediates and end-products prepared in the present invention can be purified by purification methods including but not limited to extraction, recrystallization, adsorption treatment, precipitation, reverse precipitation, membrane dialysis, supercritical extraction and the like. The characterization of the structure and molecular weight of the end-products can be confirmed by characterization methods including but not limited to NMR, electrophoresis, UV-visible spectrophotometer, FTIR, AFM, GPC, HPLC, MALDI-TOF, circular dichroism spectroscopy, and the like.

### 3.1. Cationic Liposome

In the present invention, provided herein is a cationic liposome containing any foregoing cationic lipid whose structure is represented by the general formula (1).

In one specific embodiment of the present invention, the cationic liposome preferably contains not only the cationic lipid whose structure is represented by the general formula (1), but also one or more types of lipids selected from the group consisting of neutral lipid, steroid lipid, and PEGylated lipid; more preferably simultaneously contains three types of lipids as neutral lipid, steroid lipid, and PEGylated lipid. The foregoing neutral lipid is preferably phosphoric acid.

In one specific embodiment of the present invention, neutral lipids in the cationic liposomes preferably include but are not limited to 1,2-dilinoleoyl-sn-glycero-3-phosphocholines (DLPC), 1,2-dimyristoleoyl-sn-glycero-3-phosphocholines (DMPC), 1,2-dioleoyl-sn-glycero-3-phosphocholines (DOPC), 1,2-dipalmitoyl-sn-glycero-3-phosphocholines (DPPC), 1,2-distearoyl-sn-glycero-3-phosphatidylcholines (DSPC), 1,2-diundecanoyl-sn-glycero-3-phosphatidylcholines (DUPC), 1-plamitoyl-2-oleoyl-sn-glycero-3-phosphocholines (POPC), 1,2-di-O-octadecenyl-sn-glycero-3-phosphatidylcholines (18:0 Diether PC), 1-oleoyl-2-cholesterylhemisuccinyl-sn-glycero-3-phosphocholines (OChemsPC), 1-O-hexadecyl-sn-glycero-3-phosphatidylcholines (C16 Lyso PC), 1,2-dilinolenoyl-sn-glycero-3-phosphatidylcholines, 1,2-diarachidonoyl-sn-glycero-3-phosphatidylcholines, 1,2-didecosahexaenoyl-sn-glycero-3-phosphocholines, 1,2-dioleoyl-sn-glycero-3-phosphoethanolamines (DOPE), 1,2-diphytanyl-sn-glycero-3-phosphoethanolamines (ME 16.0 PE), 1,2-distearoyl-sn-glycero-3-phosphoethanolamines, 1,2-dilinoleoyl-sn-glycero-3-phosphoethanolamines, 1,2-dilinolenoyl-sn-glycero-3-phosphoethanolamines, 1,2-diarachidonoyl-sn-glycero-3-phosphoethanolamines, 1,2-didecosahexaenoyl-sn-glycero-3-phosphoethanolamines, 1,2-dioleoyl-sn-glycero-3-phospho-rac-(1-glycerol) sodium salts (DOPG), dioleoyl phosphatidylserines (DOPS), dipalmitoylphosphatidylglycerols (DPPG), palmitoyloleoyl phosphatidylethanolamines (POPE), distearoyl phosphatidylethanolamines (DSPE), dipalmitoyl phosphatidylethanolamines (DPPE), dimyristoleoyl phosphoethanolamines (DMPE), 1-stearoyl-2-oleoyl-stearoylethanolamines (SOPE), 1-stearoyl-2-oleoyl-phosphatidylcholines (SOPC), sphingomyelins, phosphatidylcholines, phosphatidylethnolamines, phosphatidylserines, phosphatidylinositols, phosphatidic acids, palmitoyloleoyl phosphatidylcholines, lysophosphatidylcholines, lysophosphatidylethanolamines (LPE), and combinations thereof.

In one specific embodiment of the present invention, steroid lipids in the cationic liposomes are preferably selected from the group consisting of cholesterol, coprostanol, sitosterol, ergosterol, campesterol, stigmasterol, brassicasterol, tomatidine, ursolic acid, α-tocopherol, and mixtures thereof.

In one specific embodiment of the present invention, PEGylated lipids in the cationic liposomes are preferably selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-cholesterol, PEG-diacylglycamide (PEG-DAG), PEG-dialkyloxypropyl (PEG-DAA), and specifically PEG500-dipalmitoylphosphatidylcholine, PEG2000-dipalmitoylphosphatidylcholine, PEG500-stearylphosphatidylethanolamine PEG2000-distearylphosphatidylethanolamine, PEG500-1,2-oleoylphosphatidylethanolamine, PEG2000-1,2-oleoylphosphatidylethanolamine, and PEG2000-2,3-distearoylglycerol (PEG-DMG).

In one specific embodiment of the present invention, the structure of PEGylated lipid in the cationic liposomes is preferably represented by the following general formula (2):

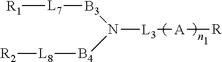

or pharmaceutically acceptable salts, tautomers, or stereoisomers thereof,
wherein, L₇ and L₈ are each independently a linking bond or a divalent linking group, said divalent linking group is selected from the group consisting of —O(C═O)—, —(C═O)O—, —O(C═O)O—, —C(═O)—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently a hydrogen atom or a C₁₋₁₂ alkyl group;
L₃ is a linking bond or a divalent linking group, when L₃ is a divalent linking group, it is selected from the divalent linking group formed by any one, two, or two more divalent linking group selected from L₄, L₅, and Z; more preferably selected from the group consisting of -L₄-, —Z-L₄-Z—, -L₄-Z-L₅-, —Z-L₄-Z-L₅-, and -L₄-Z-L₅-Z—; wherein, said L₄ and L₅ are carbon linking groups, which are each independently represented by —(CRₐR_{b})ₜ—(CRₐR_{b})ₒ—(CRₐR_{b})ₚ—, t, o, and p are each independently an integer from 0 to 12, not being 0 simultaneously; Rₐ and R_{b} are, at each occurrence, independently a hydrogen atom or a C₁₋₁₂ alkyl group; said Z is, at each occurrence, independently selected from the group consisting of —C(═O)—, —OC(═O)—, —C(═O)O—, —OC(═O)O—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently a hydrogen atom or a C₁₋₁₂ alkyl group;
B₃ and B₄ are each independently a linking bond or a C₁₋₁₂ alkylene group;
R₁ and R₂ are each independently a C₁₋₃₀ aliphatic hydrocarbon group;
R is a hydrogen atom, an alkyl group, an alkoxy group, —C(═O)R_{d}, —C(═O)OR_{d}, —OC(═O)R_{d}, —OC(═O)OR_{d}, or

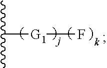

wherein, R_{d} is a C₁₋₁₂ alkyl group, G₁ is a (k+1)-valent terminal branching group, j is 0 or 1, and F contains the functional group, when j is 0, G₁ is absent; when j is 1, G₁ protrudes F with the number of k, and k is an integer from 2 to 8;
A is —(CRₐR_{b})ₛO— or —O(CRₐR_{b})ₛ—, wherein s is 2, 3 or 4, Rₐ and R_{b} are each independently a hydrogen atom or a C₁₋₁₂ alkyl group;
n₁ is an integer from 20 to 250;
said alkyl group, alkylene group, alkoxy group, and aliphatic hydrocarbon group are each independently substituted or unsubstituted.

In one specific embodiment of the present invention, the structure of a PEGylated lipid in a cationic liposome is represented by the general formula (2), and preferably selected from the group consisting of the following structures:

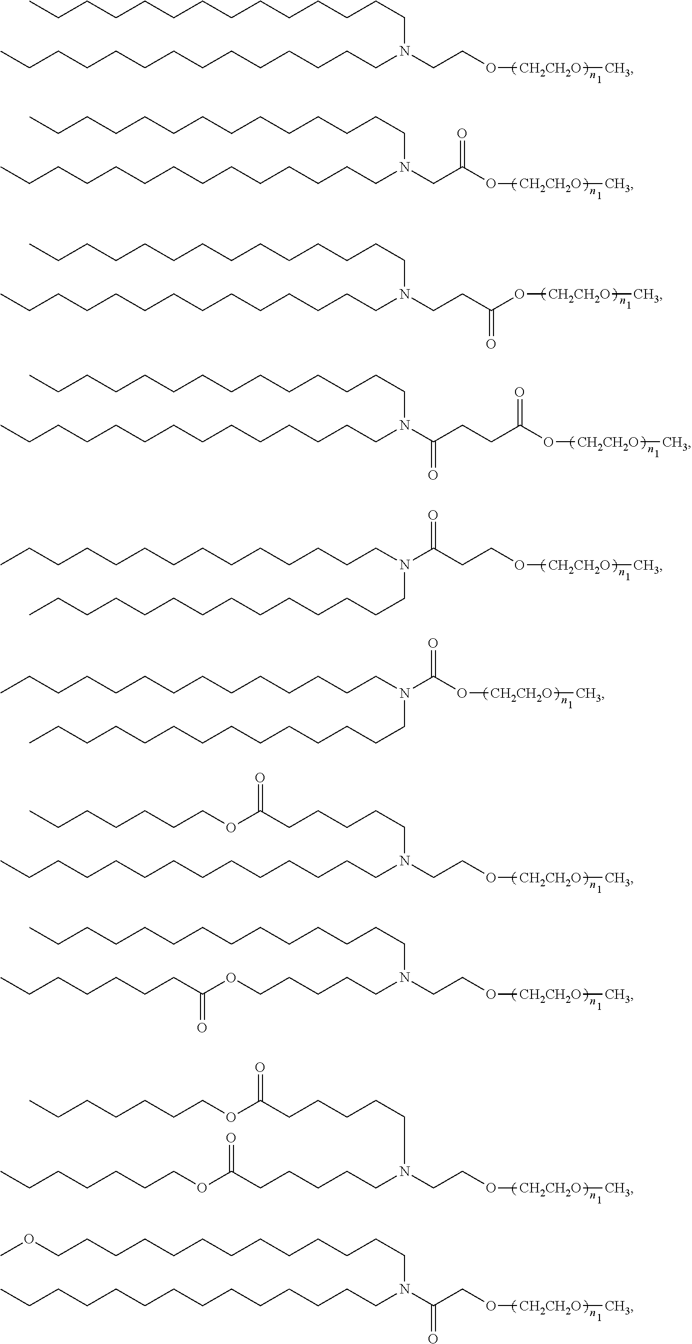

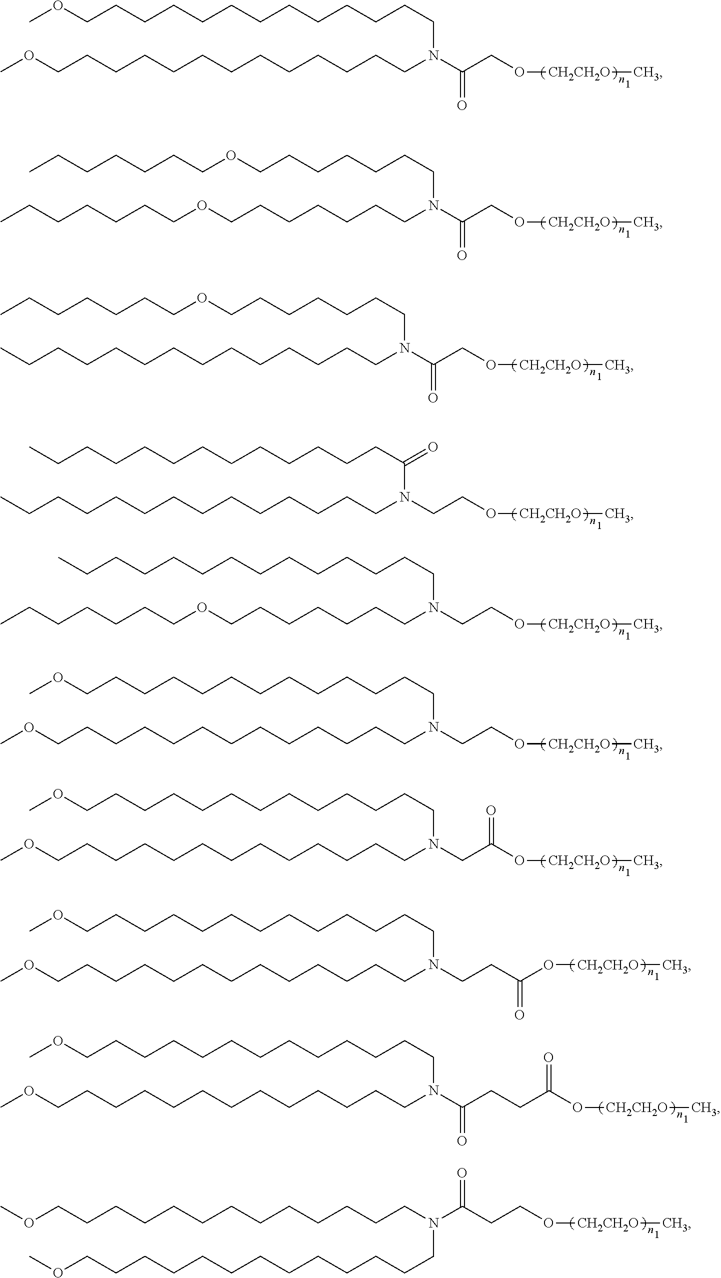

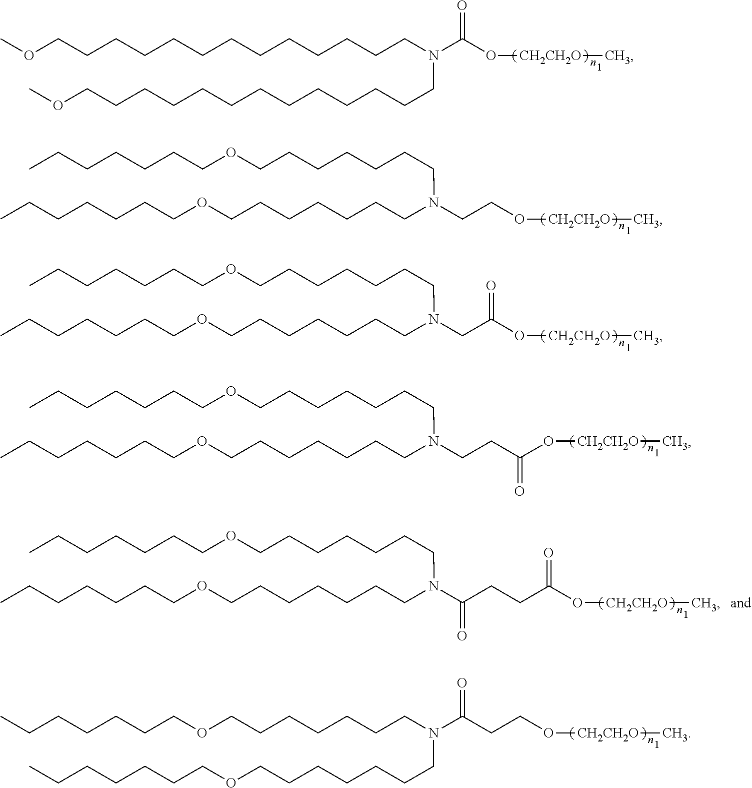

In one specific embodiment of the present invention, any foregoing cationic liposome preferably contains 20% to 80% cationic lipids represented by the general formula (1), 5% to 15% neutral lipids, 25% to 55% steroid lipids, and 0.5% to 10% PEGylated lipids, and said percentage is the molar percentage of each lipid relative to the total lipids in a solution containing the solvent.

In one specific embodiment of the present invention, in any foregoing cationic liposome, the molar percentage of cationic lipids relative to the total lipids in a solution containing the solvent is preferably 30% to 65%; more preferably about 35%, 40%, 45%, 46%, 47%, 48%, 49%, 50%, or 55%.

In one specific embodiment of the present invention, in any foregoing cationic liposomes, the molar percentage of neutral lipids relative to the total lipids in a solution containing the solvent is preferably 7.5% to 13%; more preferably about 8%, 9%, 10%, 11%, or 12%.

In one specific embodiment of the present invention, in any foregoing cationic liposomes, the molar percentage of steroid lipids relative to the total lipids in a solution containing the solvent is preferably 35% to 50%; more preferably about 40%, 41%, 42%, 43%, 44%, 45%, 46%, 47%, 48%, 49%, or 50%.

In one specific embodiment of the present invention, in any foregoing cationic liposomes, the molar percentage of PEGylated lipids relative to the total lipids in a solution containing the solvent is preferably 0.5% to 5%; preferably 1% to 3%; more preferably about 1.5%, 1.6%, 1.7%, 1.8%, or 1.9%.

### 3.2. Preparation of Cationic Liposomes

In the present invention, cationic liposomes can be prepared by the following methods, including but not limited to thin-film dispersion method, ultrasonic dispersion method, reverse phase evaporation method, freeze-drying method, freeze-thaw method, double emulsion method and/or injection method, and microfluidic method, preferably thin-film dispersion method and injection method.

### 4.1 Cationic Liposome Pharmaceutical Composition

In one embodiment of the present invention, provided herein is a cationic liposome pharmaceutical composition containing any foregoing cationic liposome and a drug, wherein the cationic liposome contains any foregoing cationic lipid whose structure is represented by the general formula (1), the drug includes but is not limited to nucleic acid drugs, genetic vaccines, antitumor drugs, small molecule drugs, polypeptide drugs, protein drugs, and the like.

In one specific embodiment of the present invention, the cationic liposome pharmaceutical composition is prepared by a simple mixing method or a microfluidic method, specifically, cationic lipids, neutral lipids, steroid lipids, and PEGylated lipids are dissolved in an organic phase according to a certain molar percentage to obtain an organic phase solution; a drug (therapeutic agent or preventive agent) is added to a water phase according to a certain N/P ratio to obtain a water phase solution; the foregoing organic phase solution and the water phase solution are mixed according to a suitable volumetric ratio (by microfluidic mixing or simple mixing); and then a post-processing purification is conducted to obtain the cationic liposome pharmaceutical composition.

In one specific embodiment of the present invention, in a cationic liposome pharmaceutical composition, the drug is preferably a nucleic acid drug, said nucleic acid drug is selected from the group consisting of RNA, DNA, antisense nucleic acid, plasmid, mRNA (messenger RNA), interfering nucleic acid, aptamer, miRNA inhibitor (antagomir), microRNA (miRNA), ribozyme, and small interfering RNA (siRNA), and preferably the group consisting of RNA, miRNA and siRNA.

In one specific embodiment of the present invention, a cationic liposome pharmaceutical composition is preferably used as a drug, including but not limited to antitumor agents, antiviral agents, antifungal agents, and vaccines.

In one specific embodiment of the present invention, the drug in cationic liposome pharmaceutical composition is nucleic acid drug, and the N/P ratio of said cationic liposomes to said nucleic acids is (0.5˜20):1, more preferably (1˜10):1, and most preferably 2:1, 4:1, 6:1, or 10:1.

In one specific embodiment of the present invention, the water phase used to dissolve the nucleic acid drugs is preferably selected from the group consisting of deionized water, ultrapure water, phosphate buffer, and physiological saline, more preferably selected from the group consisting of phosphate buffer or citrate buffer, and most preferably citrate buffer; the ratio of the cationic liposomes to the working solution is preferably (0.05˜20) g:100 mL, more preferably (0.1˜10) g:100 mL, and most preferably (0.2˜5) g:100 mL.

### 5.1 Formulation of Cationic Liposome Pharmaceutical Composition

In the present invention, provided herein is a formulation of cationic liposome pharmaceutical composition containing any foregoing cationic liposome pharmaceutical composition and pharmaceutically acceptable diluents or excipients; said diluents or excipients are preferably selected from the group consisting of deionized water, ultrapure water, phosphate buffer, and physiological saline, more preferably selected from the group consisting of phosphate buffer and physiological saline, and most preferably physiological saline.

The following is a further description with specific examples of the preparation methods of cationic lipids, cationic liposomes, and cationic liposome-nucleic acid pharmaceutical compositions, and the biological activity assays for cationic liposome-nucleic acid pharmaceutical compositions. The specific examples are for further illustration of the present invention, not limiting the protected scope of present invention. Wherein, in the embodiments of preparing cationic lipids, the structures of end-products were characterized by NMR, and the molecular weight was determined by MALDI-TOF.

### Example-1

### Example-1.1: Cationic Lipid (E1-1)

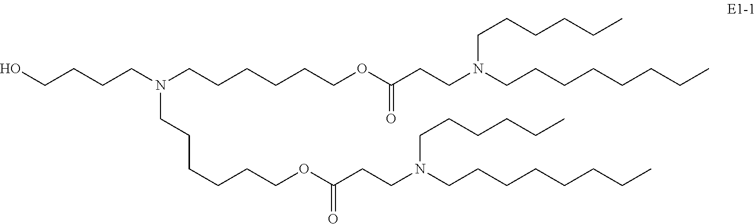 [SMILES: CCCCCCCCN(CCCCCC)CCC(=O)OCCCCCCN(CCCCO)CCCCCCOC(=O)CCN(CCCCCC)CCCCCCCC]

E1-1 corresponds to the general formula (1), wherein R₁ and R₂ are both

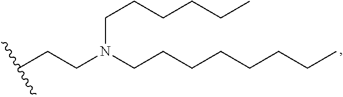

B₁ and B₂ are both hexylene groups, L₁ and L₂ are both ester groups (—C(═O)O—), X is N, L₃ is a butylene group, R₃ is a hydroxyl group, and the total molecular weight is approximately 824 Da.

The preparation process is represented as follows:

Step a: Compound N-hexyloctylamine (S1-1, 5.33 g, 25.0 mmol) was added into 100 mL anhydrous dichloromethane, and dissolved with stirring at room temperature. Potassium carbonate (K₂CO₃, 5.95 g, 50.0 mmol), 3-methylsulfonyloxypropionic acid (S1-2, 0.84 g, 5.0 mmol), and tetra-n-butylammonium bromide (0.19 g, 0.6 mmol) were added in sequence, and the reaction solution was stirred at room temperature for 72 h. After completion of the reaction, 50 mL water was added and stirred to mix, the pH was adjusted to 5-7, the solution was extracted twice with dichloromethane (50 mL*2), organic phases were combined, backwashed once with saturated NaCl solution (50 mL), and then, the organic phase was remained, dried with anhydrous sodium sulfate, filtered, and concentrated to obtain the crude product of S1-3. The crude product was purified by column chromatography, concentrated, and dried with an oil pump to obtain 3-(N-hexyloctylamino)propanoic acid (S1-3, 2.20 g).

Step b: Under argon atmosphere, into the round-bottom flask containing S1-3 (2.00 g, 7.0 mmol), 6-bromohexanol (S1-4, 1.51 g, 8.4 mmol) and 4-dimethylaminopyridine (DMAP, 0.21 g, 1.8 mmol) dissolved in dichloromethane (50 mL), N,N′-dicyclohexylcarbodiimide (DCC, 3.17 g, 15.4 mmol) was added and the reaction was conducted for 16 h at room temperature. After completion of the reaction, the precipitate was removed by filtering. The filtrate was concentrated and purified by silica gel column chromatography to obtain the bromoester S1-5 (2.55 g).

Step c: Under nitrogen protection, the compound 4-amino-1-butanol (S1-6, 0.18 g, 2.0 mmol) was dissolved in acetonitrile (50 mL), S1-5 (2.24 g, 5.0 mmol) and N,N-diisopropylethylamine (DIPEA, 0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E1-1 (1.32 g). The main data of ¹H-NMR spectrum of E1-1 were as follows: ¹H NMR (400 MHz, CDCl₃) δ: 4.06 (t, 4H), 3.64-3.61 (m, 2H), 3.24 (t, 4H), 3.03 (t, 4H), 3.02-2.81 (m, 14H), 1.80-1.21 (m, 60H), 0.87 (t, 12H). The molecular weight of E1-1 was determined to be 823.76 Da by MALDI-TOF.

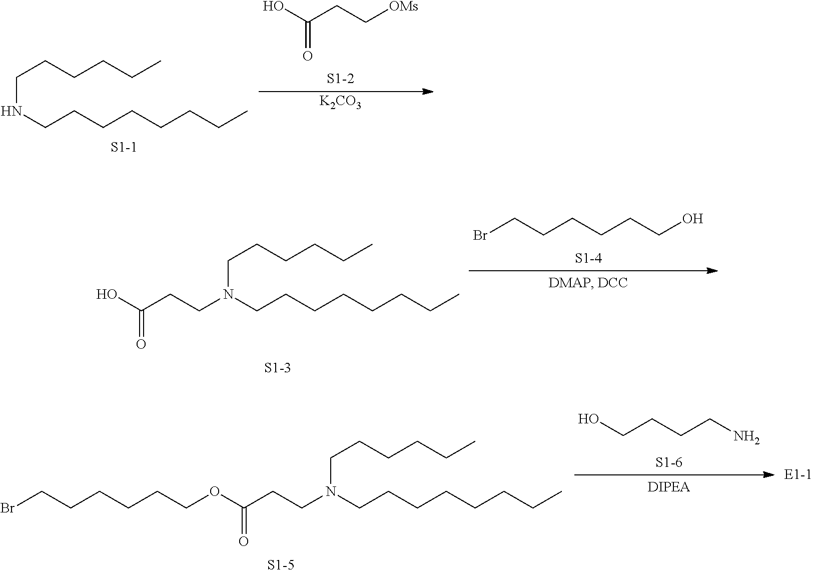

### Example-1.2: Cationic Lipid (E1-2)

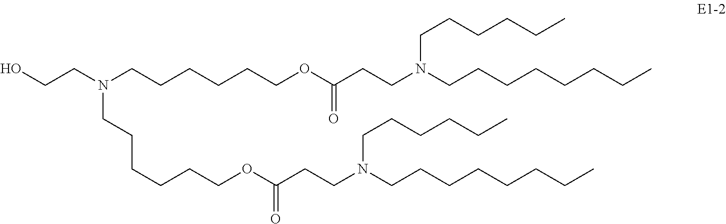 [SMILES: CCCCCCCCN(CCCCCC)CCC(=O)OCCCCCCN(CCO)CCCCCCOC(=O)CCN(CCCCCC)CCCCCCCC]

E1-2 corresponds to the general formula (1), wherein R₁ and R₂ are both

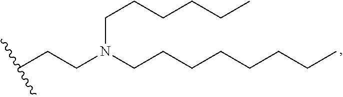

B₁ and B₂ are both hexylene groups, L₁ and L₂ are both ester groups (—C(═O)O—), X is N, L₃ is an ethylene group, R₃ is a hydroxyl group, and the total molecular weight is approximately 796 Da.

The preparation process is represented as follows:

Under nitrogen protection, the compound 2-amino-1-ethanol (S1-7, 0.12 g, 2.0 mmol) was dissolved in acetonitrile (50 mL), S1-5 (2.24 g, 5.0 mmol) and N,N-diisopropylethylamine (DIPEA, 0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E1-2 (1.27 g). The main data of ¹H-NMR spectrum of E1-2 were as follows: ¹H NMR (400 MHz, CDCl₃) δ: 4.04 (t, 4H), 3.86-3.78 (m, 2H), 3.23 (t, 4H), 3.03 (t, 4H), 3.02-2.81 (m, 14H), 1.81-1.22 (m, 56H), 0.87 (t, 12H). The molecular weight of E1-2 was determined to be 795.75 Da by MALDI-TOF.

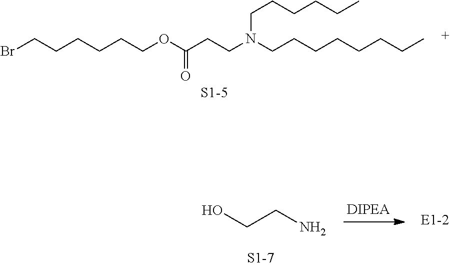

### Example-1.3: Cationic Lipid (E1-3)

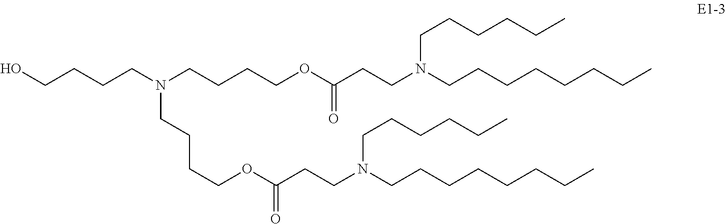 [SMILES: CCCCCCCCN(CCCCCC)CCC(=O)OCCCCN(CCCCO)CCCCOC(=O)CCN(CCCCCC)CCCCCCCC]

E1-3 corresponds to the general formula (1), wherein R₁ and R₂ are both

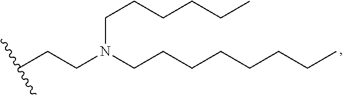

B₁ and B₂ are both butylene groups, L₁ and L₂ are both ester groups (—C(═O)O—), X is N, L₃ is a butylene group, R₃ is a hydroxyl group, and the total molecular weight is approximately 768 Da.

The preparation process is represented as follows:

Step a: Under argon atmosphere, into the round-bottom flask containing S1-3 (2.85 g, 10.0 mmol), 4-bromo-n-butanol (S1-8, 1.82 g, 12.0 mmol) and DMAP (0.31 g, 2.5 mmol) dissolved in dichloromethane (150 mL), DCC (4.53 g, 22.0 mmol) was added and the reaction was conducted for 16 h at room temperature. After completion of the reaction, the precipitate was removed by filtering. The filtrate was concentrated and purified by silica gel column chromatography to obtain the bromoester S1-9 (3.59 g).

Step b: Under nitrogen protection, the compound S1-6 (0.18 g, 2.0 mmol) was dissolved in acetonitrile (50 mL), S1-9 (2.10 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E1-3 (1.23 g). The main data of ¹H-NMR spectrum of E1-3 were as follows: ¹H NMR (400 MHz, CDCl₃) δ: 4.07 (t, 4H), 3.64-3.61 (m, 2H), 3.24 (t, 4H), 3.03 (t, 4H), 3.02-2.81 (m, 14H), 1.81-1.22 (m, 52H), 0.87 (t, 12H). The molecular weight of E1-3 was determined to be 767.70 Da by MALDI-TOF.

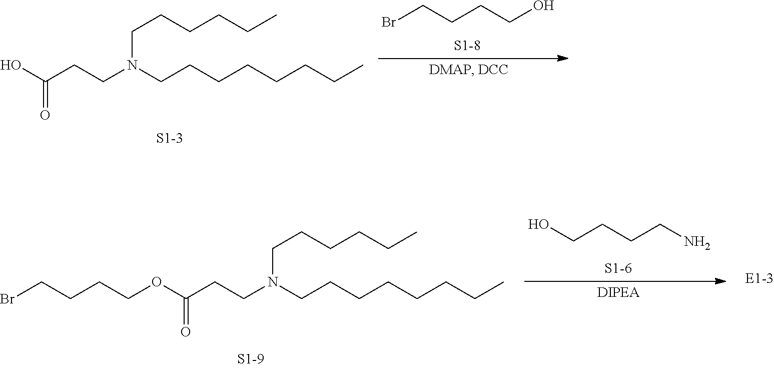 [SMILES: CCCCCCCCN(CCCCCC)CCC(=O)O ; OCCCCBr ; CCCCCCCCN(CCCCCC)CCC(=O)OCCCCBr ; NCCCCO]

### Example-2: Cationic Lipid (E2-1)

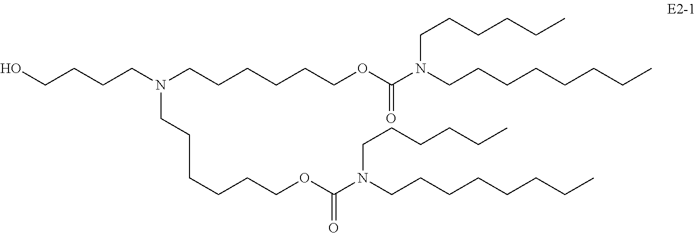 [SMILES: CCCCCCCCN(CCCCCC)C(=O)OCCCCCCN(CCCCO)CCCCCCOC(=O)N(CCCCCC)CCCCCCCC]

E2-1 corresponds to the general formula (1), wherein R₁ and R₂ are both

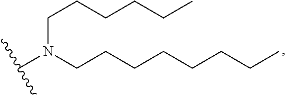

B₁ and B₂ are both hexylene groups, L₁ and L₂ are both ester groups (—C(═O)O—), X is N, L₃ is a butylene group, R₃ is a hydroxyl group, and the total molecular weight is approximately 768 Da.

The preparation process is represented as follows:

Step a: Compound S1-1 (2.57 g, 12.0 mmol) was dissolved in dichloromethane (50 mL), and then 6-bromohexyl-N-succinimide carbonate (S2-1, 3.22 g, 10.0 mmol) and triethylamine (TEA, 1.10 mL, 15.0 mmol) were added successively, and the reaction was stirred at room temperature overnight. After completion of the reaction, the reaction solution was concentrated to obtain the crude product. The crude product was purified by column chromatography, concentrated, and dried with an oil pump to obtain the bromoester S2-2 (3.31 g).

Step b: Under nitrogen protection, the compound S1-6 (0.18 g, 2.0 mmol) was dissolved in acetonitrile (50 mL), S2-2 (2.10 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E2-1 (1.25 g). The main data of ¹H-NMR spectrum of E2-1 were as follows: ¹H NMR (400 MHz, CDCl₃) δ: 4.05 (t, 4H), 3.65-3.61 (m, 2H), 3.22-3.06 (m, 8H), 2.91-2.63 (m, 6H), 1.81-1.22 (m, 60H), 0.88 (t, 12H). The molecular weight of E2-1 was determined to be 767.73 Da by MALDI-TOF.

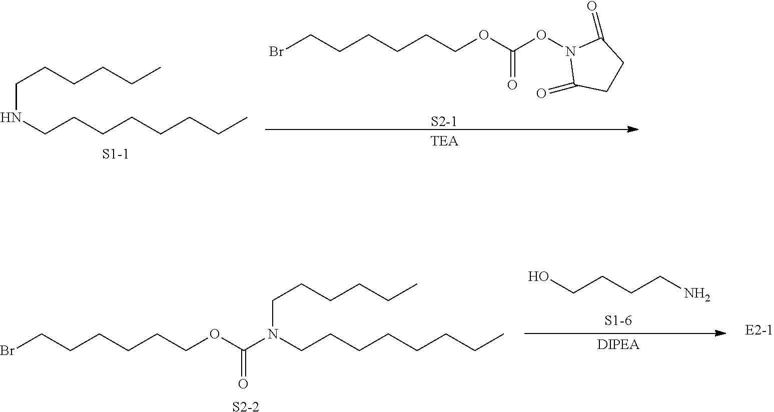

### Example-3: Cationic Lipid (E3-1)

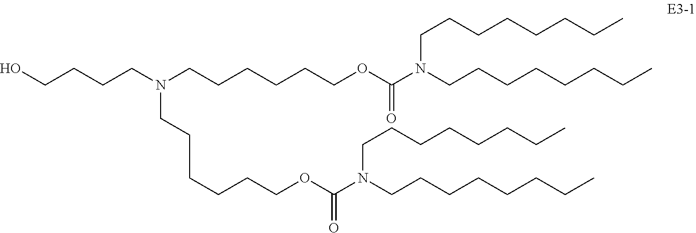 [SMILES: CCCCCCCCN(CCCCCCCC)C(=O)OCCCCCCN(CCCCO)CCCCCCOC(=O)N(CCCCCCCC)CCCCCCCC]

E3-1 corresponds to the general formula (1), wherein R₁ and R₂ are both

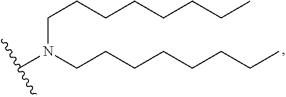

B₁ and B₂ are both hexylene groups, L₁ and L₂ are both ester groups (—C(═O)O—), X is N, L₃ is a butylene group, R₃ is a hydroxyl group, and the total molecular weight is approximately 824 Da.

The preparation process is represented as follows:

Step a: Compound S3-1 (2.89 g, 12.0 mmol) was dissolved in dichloromethane (50 mL), and then S2-1 (3.22 g, 10.0 mmol) and TEA (1.10 mL, 15.0 mmol) were added successively, the reaction was stirred at room temperature overnight. After completion of the reaction, the reaction solution was concentrated to obtain the crude product. The crude product was purified by column chromatography, concentrated, and dried with an oil pump to obtain the bromoester S3-2 (3.49 g).

Step b: Under nitrogen protection, the compound S1-6 (0.18 g, 2.0 mmol) was dissolved in acetonitrile (50 mL), S3-2 (2.24 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E3-1 (1.35 g). The main data of ¹H-NMR spectrum of E3-1 were as follows: ¹H NMR (400 MHz, CDCl₃) δ: 4.03 (t, 4H), 3.64-3.61 (m, 2H), 3.20-3.06 (m, 8H), 2.91-2.59 (m, 6H), 1.81-1.22 (m, 68H), 0.85 (t, 12H). The molecular weight of E3-1 was determined to be 832.75 Da by MALDI-TOF.

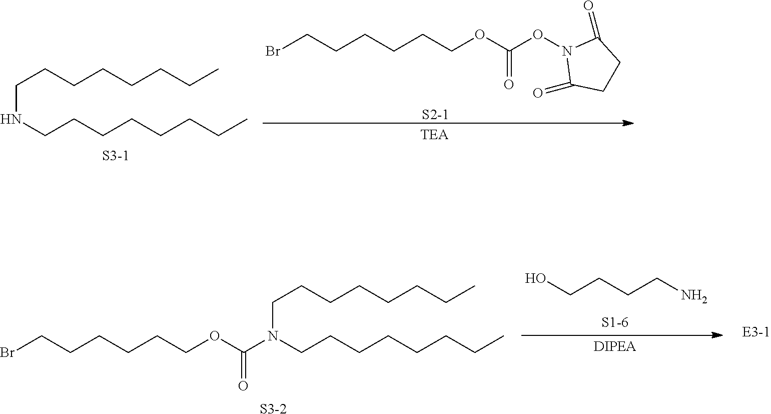

### Example-4: Cationic Lipid (E4-1)

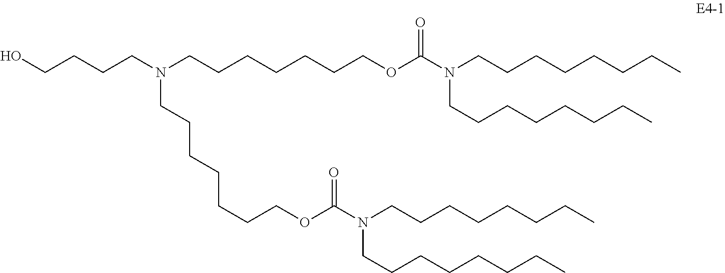 [SMILES: CCCCCCCCN(CCCCCCCC)C(=O)OCCCCCCCN(CCCCO)CCCCCCCOC(=O)N(CCCCCCCC)CCCCCCCC]

E4-1 corresponds to the general formula (1), wherein R₁ and R₂ are both

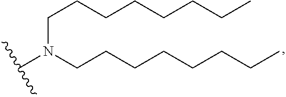

B₁ and B₂ are both heptylene groups, L₁ and L₂ are both ester groups (—C(═O)O—), X is N, L₃ is a butylene group, R₃ is a hydroxyl group, and the total molecular weight is approximately 852 Da.

The preparation process is represented as follows:

Step a: Compound S3-1 (2.89 g, 12.0 mmol) was dissolved in dichloromethane (50 mL), and then 7-bromoheptyl-N-succinimide carbonate (S4-1, 3.36 g, 10.0 mmol) and TEA (1.10 mL, 15.0 mmol) were added successively, and the reaction was stirred at room temperature overnight. After completion of the reaction, the reaction solution was concentrated to obtain the crude product. The crude product was purified by column chromatography, concentrated, and dried with an oil pump to obtain the bromoester S4-2 (3.61 g).

Step b: Under nitrogen protection, the compound S1-6 (0.18 g, 2.0 mmol) was dissolved in acetonitrile (50 mL), S4-2 (2.24 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E4-1 (1.40 g). The main data of ¹H-NMR spectrum of E4-1 were as follows: ¹H NMR (400 MHz, CDCl₃) δ: 4.05 (t, 4H), 3.64-3.61 (m, 2H), 3.24-3.06 (m, 8H), 2.90-2.61 (m, 6H), 1.82-1.20 (m, 72H), 0.86 (t, 12H). The molecular weight of E4-1 was determined to be 851.83 Da by MALDI-TOF.

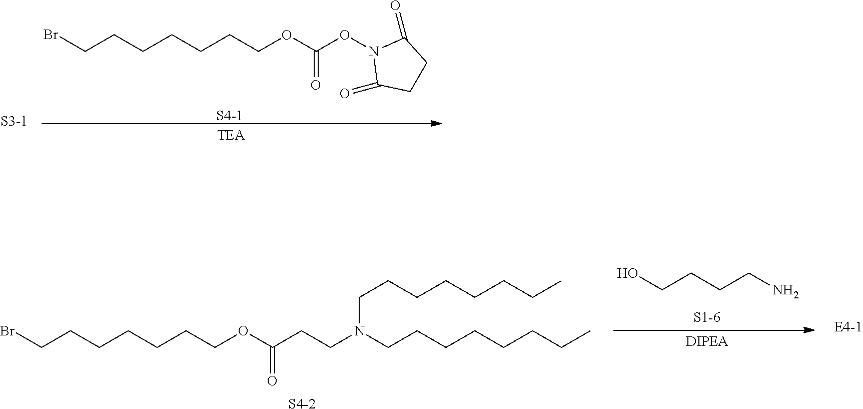

### Example-5: Cationic Lipid (E5-1)

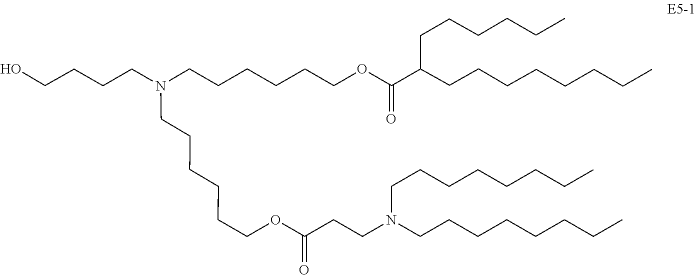 [SMILES: CCCCCCCCC(CCCCCC)C(=O)OCCCCCCN(CCCCO)CCCCCCOC(=O)CCN(CCCCCCCC)CCCCCCCC]

E5-1 corresponds to the general formula (1), wherein R₁ is

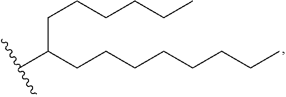

### R₂ is

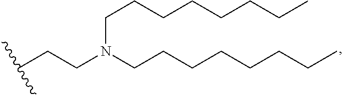 [SMILES: *CCN(CCCCCCCC)CCCCCCCC]

B₁ and B₂ are both hexylene groups, L₁ and L₂ are both ester groups (—C(═O)O—), X is N, L₃ is a butylene group, R₃ is a hydroxyl group, and the total molecular weight is approximately 823 Da.

The preparation process is represented as follows:

Step a: Under nitrogen atmosphere, into the round-bottom flask containing 2-hexyldecanoic acid (S5-1, 2.56 g, 10.0 mmol), S1-4 (2.16 g, 12.0 mmol) and DMAP (0.31 g, 2.5 mmol) dissolved in dichloromethane (100 mL), DCC (4.53 g, 22.0 mmol) was added and the reaction was conducted for 16 h at room temperature. After completion of the reaction, the precipitate was removed by filtering. The filtrate was concentrated and purified by silica gel column chromatography to obtain the bromoester S5-2 (3.39 g).

Step b: Under nitrogen protection, the compound S1-6 (0.36 g, 4.0 mmol) was dissolved in acetonitrile (50 mL), S5-2 (2.10 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S5-3 (1.40 g).

Step c: Under nitrogen protection, the compound S5-3 (0.86 g, 2.0 mmol) was dissolved in acetonitrile (30 mL), S5-4 (1.19 g, 2.5 mmol, wherein S5-4 is a product of a reaction between S1-4 and

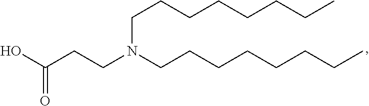 [SMILES: CCCCCCCCN(CCCCCCCC)CCC(=O)O]

referring to Step b in Example-1.1 for specific experimental steps) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E5-1 (1.34 g). The main data of ¹H-NMR spectrum of E5-1 were as follows: ¹H NMR (400 MHz, CDCl₃) δ: 4.06 (t, 2H), 4.01 (t, 2H), 3.66-3.62 (m, 2H), 3.22 (t, 2H), 3.04 (t, 2H), 3.02-2.81 (m, 10H), 2.29-2.22 (m, 1H), 1.92-1.21 (m, 68H), 0.83 (t, 12H). The molecular weight of E5-1 was determined to be 822.77 Da by MALDI-TOF.

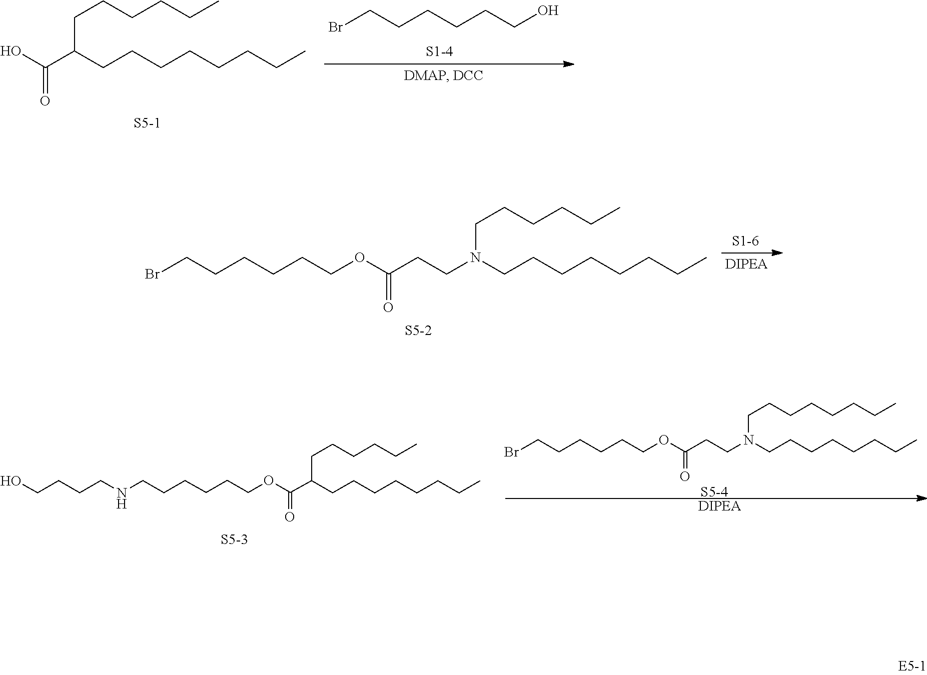

### Example-6.1: Cationic Lipid (E6-1)

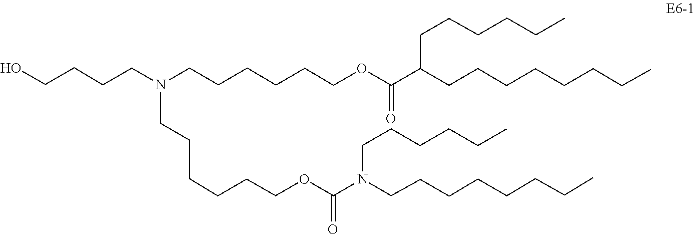 [SMILES: CCCCCCCCC(CCCCCC)C(=O)OCCCCCCN(CCCCO)CCCCCCOC(=O)N(CCCCCC)CCCCCCCC]

E6-1 corresponds to the general formula (1), wherein R₁ is

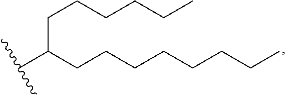

### R₂ is

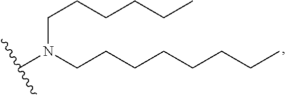 [SMILES: *N(CCCCCC)CCCCCCCC]

B₁ and B₂ are both hexylene groups, L₁ and L₂ are both ester groups (—C(═O)O—), X is N, L₃ is a butylene group, R₃ is a hydroxyl group, and the total molecular weight is approximately 767 Da.

The preparation process is represented as follows:

Under nitrogen protection, the compound S5-3 (0.86 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E6-1 (1.25 g). The main data of ¹H-NMR spectrum of E6-1 were as follows: ¹H NMR (400 MHz, CDCl₃) δ: 4.06-4.00 (m, 4H), 3.65 (t, 2H), 3.22-3.09 (m, 4H), 2.90-2.75 (m, 6H), 2.33-2.22 (m, 1H), 1.83-1.22 (m, 64H), 0.86 (t, 12H). The molecular weight of E6-1 was determined to be 766.87 Da by MALDI-TOF.

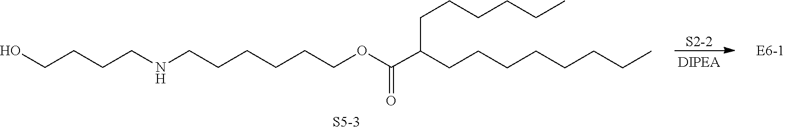

### Example-6.2: Cationic Lipid (E6-2)

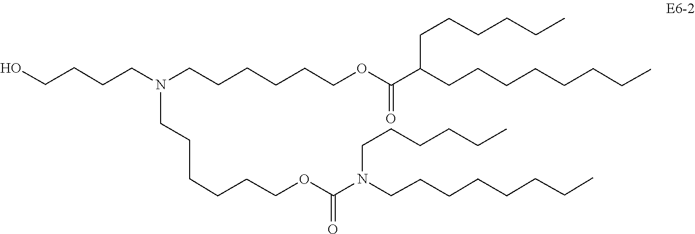 [SMILES: CCCCCCCCC(CCCCCC)C(=O)OCCCCCCN(CCCCO)CCCCCCOC(=O)N(CCCCCC)CCCCCCCC]

E6-2 corresponds to the general formula (1), wherein R₁ is

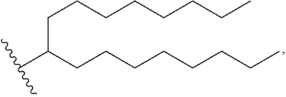

### R₂ is

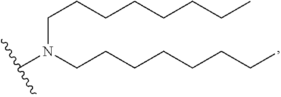

B₁ and B₂ are both hexylene groups, L₁ and L₂ are both ester groups (—C(═O)O—), X is N, L₃ is a butylene group, R₃ is a hydroxyl group, and the total molecular weight is approximately 823 Da.

The preparation process is represented as follows:

According to the method of Step a and Step b in Example-5, the starting material 2-hexyldecanoic acid in Step a was replaced with 2-octyldecanoic acid to prepare the S6-1, and then the reaction between S6-1 and S3-2 was carried out according to the dosing amount and operation procedure in Example-6.1 to obtain the cationic lipid E6-2. The main data of ¹H-NMR spectrum of E6-2 were as follows: ¹H NMR (400 MHz, CDCl₃) δ: 4.06-4.02 (m, 4H), 3.65 (t, 2H), 3.22-3.10 (m, 4H), 2.92-2.76 (m, 6H), 2.33-2.24 (m, 1H), 1.79-1.22 (m, 72H), 0.85 (t, 12H). The molecular weight of E6-2 was determined to be 822.62 Da by MALDI-TOF.

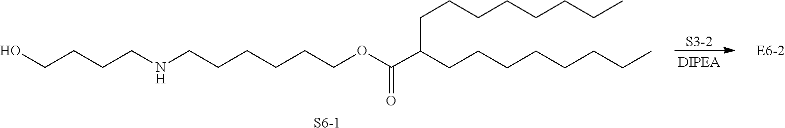

### Example-7.1: Cationic Lipid (E7-1)

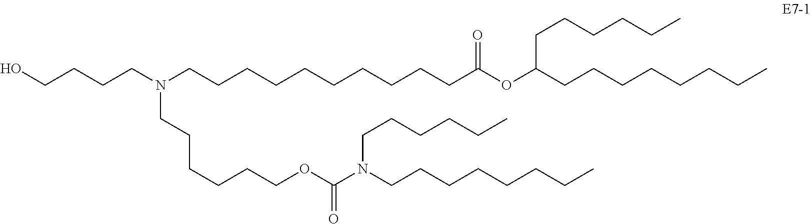 [SMILES: CCCCCCCCC(CCCCCC)OC(=O)CCCCCCCCCCN(CCCCO)CCCCCCOC(=O)N(CCCCCC)CCCCCCCC]

E7-1 corresponds to the general formula (1), wherein R₁ is

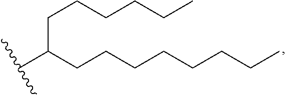

### R₂ is

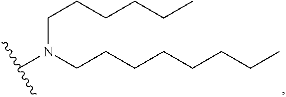 [SMILES: *N(CCCCCC)CCCCCCCC]

B₁ and B₂ are both hexylene groups, L₁ is an ester group (—OC(═O)O—), L₂ is an ester group (—C(═O)O—), X is N, L₃ is a butylene group, R₃ is a hydroxyl group, and the total molecular weight is approximately 767 Da.

The preparation process is represented as follows:

Step a: Under nitrogen atmosphere, into the round-bottom flask containing S5-1 (2.08 g, 10.0 mmol), 7-pentadecanol (S7-2, 2.74 g, 12.0 mmol) and DMAP (0.31 g, 2.5 mmol) dissolved in dichloromethane (100 mL), DCC (4.53 g, 22.0 mmol) was added and the reaction was conducted for 16 h at room temperature. After completion of the reaction, the precipitate was removed by filtering. The filtrate was concentrated and purified by silica gel column chromatography to obtain the bromoester (S7-3, 3.47 g).

Step b: Under nitrogen protection, the compound S1-6 (0.36 g, 4.0 mmol) was dissolved in acetonitrile (50 mL), S7-3 (2.10 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S7-4 (1.40 g).

Step c: Under nitrogen protection, the compound S7-4 (0.86 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E7-1 (1.24 g). The main data of ¹H-NMR spectrum of E7-1 were as follows: ¹H NMR (400 MHz, CDCl₃) δ: 4.87-4.79 (m, 1H), 4.03-3.99 (t, 2H), 3.64-3.58 (m, 2H), 3.24-3.06 (m, 4H), 2.90-2.59 (m, 6H), 2.30-2.24 (t, 2H), 1.81-1.22 (m, 64H), 0.85 (t, 12H). The molecular weight of E7-1 was determined to be 766.71 Da by MALDI-TOF.

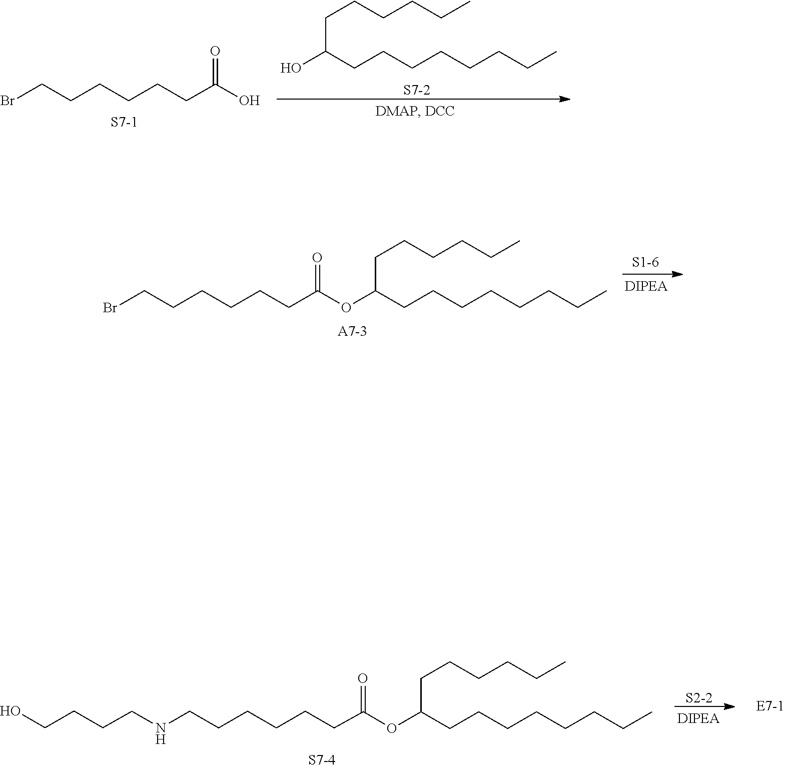

### Example-7.2: Cationic Lipid (E7-2)

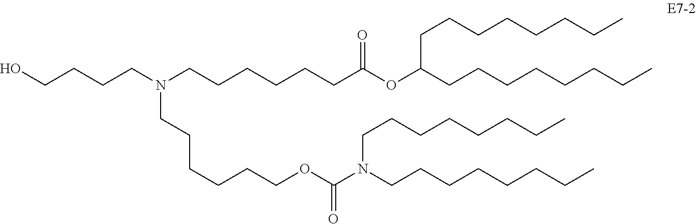 [SMILES: CCCCCCCCC(CCCCCCCC)OC(=O)CCCCCCN(CCCCO)CCCCCCOC(=O)N(CCCCCCCC)CCCCCCCC]

E7-2 corresponds to the general formula (1), wherein R₁ is

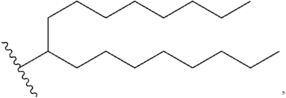

### R₂ is

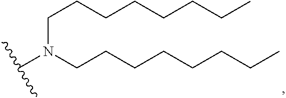 [SMILES: *N(CCCCCCCC)CCCCCCCC]

B₁ and B₂ are both hexylene groups, L₁ is an ester group (—OC(═O)O—), L₂ is an ester group (—C(═O)O—), X is N, L₃ is a butylene group, R₃ is a hydroxyl group, and the total molecular weight is approximately 823 Da.

The preparation process is represented as follows:

According to the method of Step a and Step b in Example-7, the starting material 7-pentadecanol in Step a was replaced with 9-heptadecanol to prepare the S7-5, and then the reaction between S7-5 and S3-2 was carried out according to the dosing amount and operation procedure in Example-7.1 to obtain the cationic lipid E7-2. The main data of ¹H-NMR spectrum of E7-2 were as follows: ¹H NMR (400 MHz, CDCl₃) δ: 4.86-4.80 (m, 1H), 4.02-3.98 (t, 2H), 3.64-3.60 (m, 2H), 3.26-3.08 (m, 4H), 2.90-2.62 (m, 6H), 2.31-2.25 (t, 2H), 1.80-1.22 (m, 72H), 0.85 (t, 12H). The molecular weight of E7-2 was determined to be 822.68 Da by MALDI-TOF.

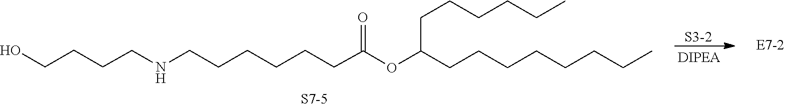

### Example-8: Cationic Lipid (E8-1)

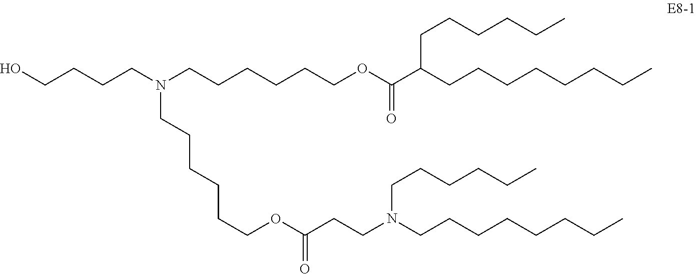 [SMILES: CCCCCCCCC(CCCCCC)C(=O)OCCCCCCN(CCCCO)CCCCCCOC(=O)CCN(CCCCCC)CCCCCCCC]

E8-1 corresponds to the general formula (1), wherein R₁ is

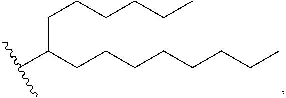

### R₂ is

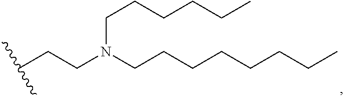 [SMILES: *CCN(CCCCCC)CCCCCCCC]

B₁ and B₂ are both hexylene groups, L₁ and L₂ are both ester groups (—C(═O)O—), X is N, L₃ is a butylene group, R₃ is a hydroxyl group, and the total molecular weight is approximately 795 Da.

The preparation process is represented as follows:

Under nitrogen protection, the compound S5-3 (0.86 g, 2.0 mmol) was dissolved in acetonitrile (30 mL), S1-5 (1.12 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E8-1 (1.29 g). The main data of ¹H-NMR spectrum of E8-1 were as follows: ¹H NMR (400 MHz, CDCl₃) δ: 4.07 (t, 2H), 4.01 (t, 2H), 3.67-3.62 (m, 2H), 3.22 (t, 2H), 3.04 (t, 2H), 2.99-2.85 (m, 10H), 2.30-2.21 (m, 1H), 1.93-1.47 (m, 20H), 1.45-1.16 (m, 44H), 0.84 (t, 12H).

The molecular weight of E8-1 was determined to be 793.83 Da by MALDI-TOF.

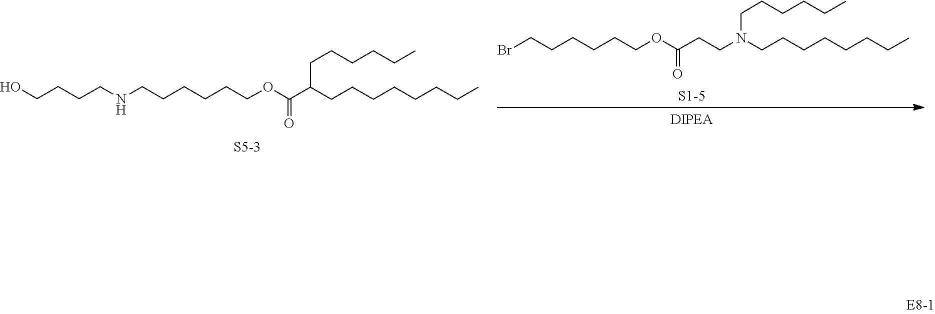

### Example-9: Cationic Lipid (E9-1)

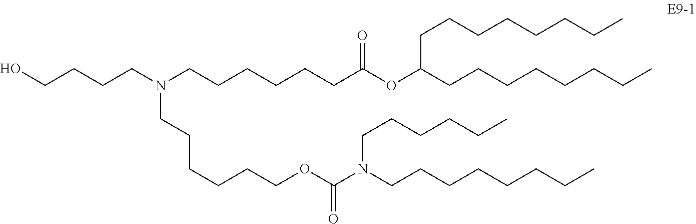 [SMILES: CCCCCCCCC(CCCCCCCC)OC(=O)CCCCCCN(CCCCO)CCCCCCOC(=O)N(CCCCCC)CCCCCCCC]

E9-1 corresponds to the general formula (1), wherein R₁ is

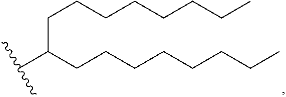

### R₂ is

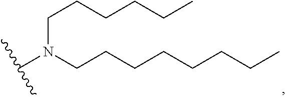 [SMILES: *N(CCCCCC)CCCCCCCC]

B₁ and B₂ are both hexylene groups, L₁ is an ester group (—OC(═O)—), L₂ is an ester group (—C(═O)O—), X is N, L₃ is a butylene group, R₃ is a hydroxyl group, and the total molecular weight is approximately 795 Da.

The preparation process is represented as follows:

Step a: Under nitrogen protection, the compound S1-6 (0.36 g, 4.0 mmol) was dissolved in acetonitrile (50 mL), S9-1 (2.24 g, 5.0 mmol, wherein S9-1 is a product of a reaction between S6-1 and

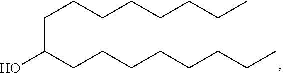 [SMILES: CCCCCCCCC(O)CCCCCCCC]

referring to Step b in Example-6 for specific experimental steps) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid S9-2 (1.48 g).

Step b: Under nitrogen protection, the compound S9-2 (0.91 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E9-1 (1.30 g). The main data of ¹H-NMR spectrum of E9-1 were as follows: ¹H NMR (400 MHz, CDCl₃) δ: 4.87-4.78 (m, 1H), 4.03-3.99 (t, 2H), 3.58 (t, 2H), 3.24-3.06 (m, 4H), 2.90-2.59 (m, 6H), 2.30-2.24 (t, 2H), 1.81-1.22 (m, 68H), 0.85 (t, 12H). The molecular weight of E9-1 was determined to be 794.74 Da by MALDI-TOF.

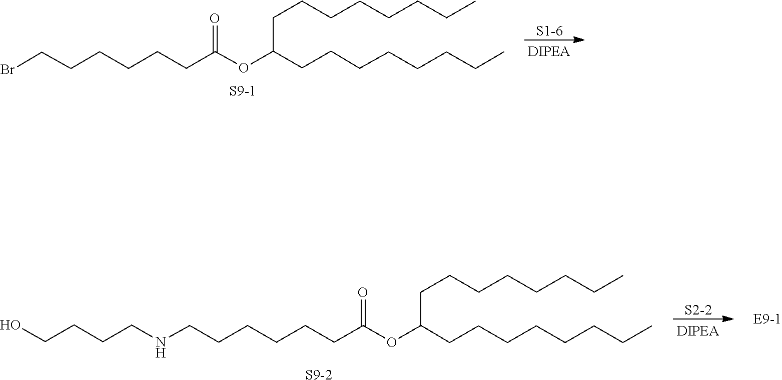

### Example-10: Cationic Lipid (E10-1)

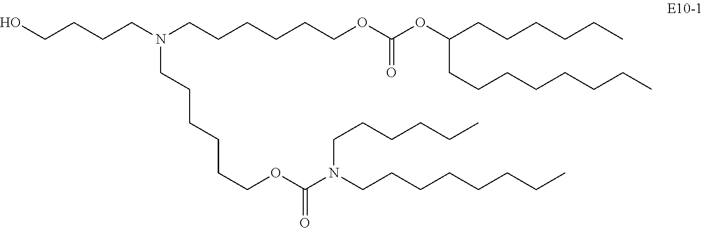 [SMILES: CCCCCCCCC(CCCCCC)OC(=O)OCCCCCCN(CCCCO)CCCCCCOC(=O)N(CCCCCC)CCCCCCCC]

E10-1 corresponds to the general formula (1), wherein R₁ is

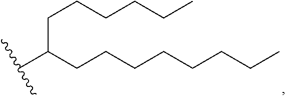

### R₂ is

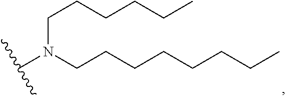 [SMILES: *N(CCCCCC)CCCCCCCC]

B₁ and B₂ are both hexylene groups, L₁ is a carbonate group (—OC(═O)O—), L₂ is an ester group (—C(═O)O—), X is N, L₃ is a butylene group, R₃ is a hydroxyl group, and the total molecular weight is approximately 783 Da.

The preparation process is represented as follows:

Step a: Under nitrogen protection, 6-bromohexyl-4-nitrophenyl carbonate (S10-1, 3.45 g, 10.0 mmol, wherein S10-1 is a product of a reaction between p-nitrophenyl chloroformate and 6-bromohexanol) was dissolved in dichloromethane (300 mL), S6-2 (9.12 g, 40.0 mmol) was added dropwise slowly with stirring at room temperature, pyridine (1.00 mL, 12.5 mmol) was then added dropwise slowly over 10 min, followed by a single addition of DMAP (0.24 g, 2.0 mmol). The reaction was stirred at room temperature for 16 h, after completion of the reaction, the reaction solution was extracted twice with dichloromethane, the organic phases were combined and washed with saline, dried with anhydrous MgSO₄, and filtered to obtain the crude product. The crude product was purified by silica gel column. The target eluent was collected and concentrated to obtain the oxidation product S10-2 (1.21 g).

Step b: Under nitrogen protection, the compound S1-6 (0.18 g, 2.0 mmol) was dissolved in acetonitrile (50 mL), S10-2 (1.06 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S10-3 (0.72 g).

Step c: Under nitrogen protection, the compound S10-3 (0.44 g, 1.0 mmol) was dissolved in acetonitrile (20 mL), S2-2 (0.52 g, 1.3 mmol) and DIPEA (0.09 g, 1.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E10-1 (0.64 g). The main data of ¹H-NMR spectrum of E10-1 were as follows: ¹H NMR (400 MHz, CDCl₃) δ: 4.71-4.68 (m, 1H), 4.21 (t, 2H), 4.03 (t, 2H), 3.68-3.60 (t, 2H), 2.55-2.46 (m, 10H), 1.75-1.25 (m, 64H), 0.89 (t, 12H). The molecular weight of E10-1 was determined to be 782.72 Da by MALDI-TOF.

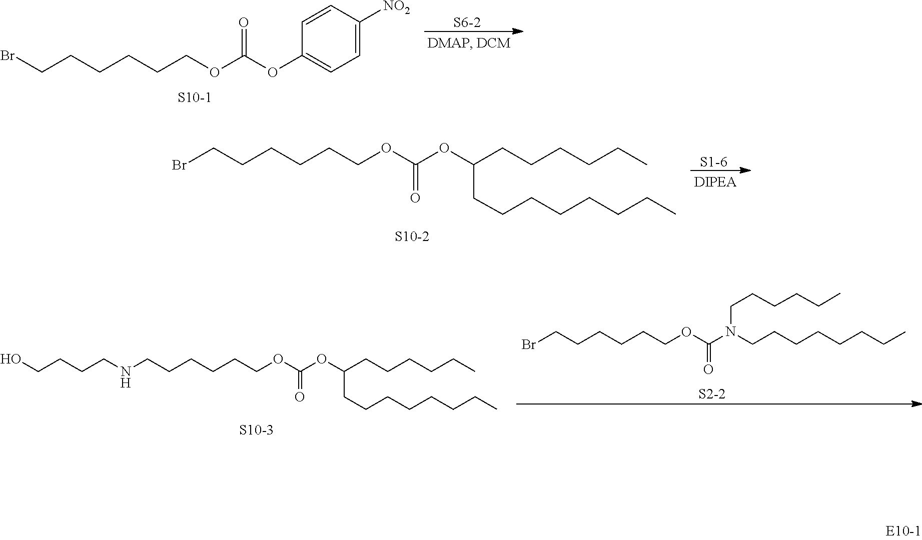

### Example-11: Cationic Lipid (E11-1)

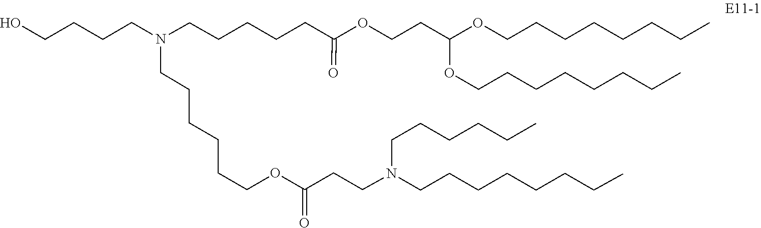 [SMILES: CCCCCCCCOC(CCOC(=O)CCCCCN(CCCCO)CCCCCCOC(=O)CCN(CCCCCC)CCCCCCCC)OCCCCCCCC]

E11-1 corresponds to the general formula (1), wherein R₁ is

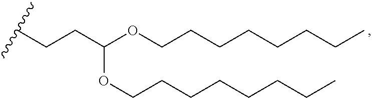

### R₂ is

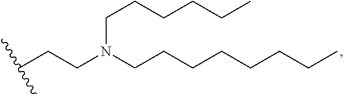 [SMILES: *CCN(CCCCCC)CCCCCCCC]

B₁ is a pentylene group, B₂ is a hexylene group, L₁ is an ester group (—OC(═O)—), L₂ is an ester group (—C(═O)O—), X is N, L₃ is a butylene group, R₃ is a hydroxyl group, and the total molecular weight is approximately 869 Da.

The preparation process is represented as follows:

Step a: 1,3-propanediol (S11-1, 9.50 g, 50 mmol) containing a TBS protected hydroxy group was dissolved in 400 mL dichloromethane. Pyridinium chlorochromate (PCC, 16.13 g, 75.0 mmol) was added and the reaction was conducted for at least 2 h at 15° C. After completion of the reaction, the reaction solution was filtered, concentrated under reduced pressure, and purified by silica gel column chromatography to obtain 3-hydroxypropionic acid with TBS protected hydroxy group (S11-2, 6.02 g).

Step b: Compound S11-2 (5.64 g, 30.0 mmol) and 1-octanol (S11-3, 9.75 g, 75.0 mmol) were dissolved in 200 mL dichloromethane, followed by adding p-toluenesulfonic acid monohydrate (TsOH H₂O, 1.14 g, 6.0 mmol) and anhydrous sodium sulfate (10.65 g, 75.0 mmol). After the reaction was conducted for at least 24 h at 15° C., the solution was filtered and concentrated under reduced pressure to obtain the crude product. The crude product was purified by silica gel column chromatography to obtain the acetal with TBS protected hydroxy group (S11-4, 2.84 g).

Step c: Into a flask under nitrogen protection, the above product S11-4 (2.16 g, 5.0 mmol) was dissolved in THE (50 mL), tetrabutylammonium fluoride solution (TBAF, 50 mL, 1M) was added, and the reaction was conducted overnight to remove the TBS protecting group. The reaction solution was dried with anhydrous sodium sulfate, filtered, and concentrated to obtain the crude product of S11-5. The crude product was purified by column chromatography, concentrated, and dried with an oil pump to obtain the acetal with naked hydroxy group S11-5 (1.40 g, 88.6%).

Step d: Under argon atmosphere, into the round-bottom flask containing S11-5 (0.76 g, 2.4 mmol), S11-6 (0.39 g, 2.0 mmol) and DMAP (61.00 mg, 0.5 mmol) dissolved in dichloromethane (100 mL), DCC (0.91 g, 4.4 mmol) was added and the reaction was conducted for 16 h at room temperature. After completion of the reaction, the precipitate was removed by filtering. The filtrate was concentrated and purified by silica gel column chromatography to obtain the bromoester S11-7 (0.81 g).

Step e: Under nitrogen protection, the compound S1-6 (0.09 g, 1.0 mmol) was dissolved in acetonitrile (20 mL), S11-7 (0.62 g, 1.3 mmol) and DIPEA (0.09 g, 1.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S11-8 (0.42 g).

Step f: Under nitrogen protection, the compound S11-8 (0.30 g, 0.6 mmol) was dissolved in acetonitrile (20 mL), S1-5 (0.34 g, 0.8 mmol) and DIPEA (0.05 g, 0.6 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E11-1 (0.43 g). The main data of ¹H-NMR spectrum of E11-1 were as follows: ¹H NMR (400 MHz, CDCl₃) δ: 4.64 (t, 1H), 4.06 (t, 4H), 3.64-3.61 (m, 4H), 3.52-3.36 (m, 4H), 3.02-2.81 (m, 10H), 2.32 (t, 4H), 1.80-1.21 (m, 64H), 0.87 (t, 12H). The molecular weight of E11-1 was determined to be 868.79 Da by MALDI-TOF.

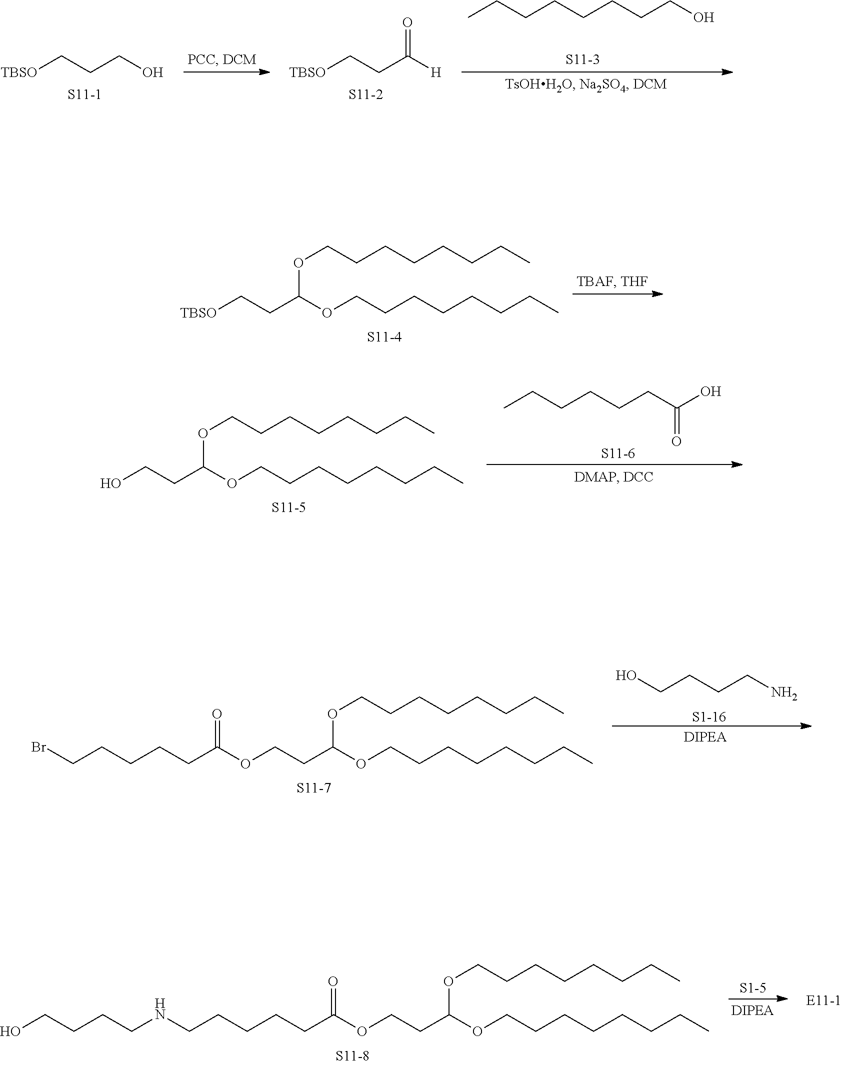

### Example-12: Cationic Lipid (E12-1)

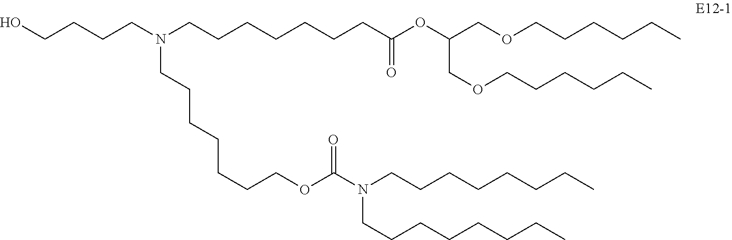 [SMILES: CCCCCCCCN(CCCCCCCC)C(=O)OCCCCCCCN(CCCCO)CCCCCCCC(=O)OC(COCCCCCC)COCCCCCC]

E12-1 corresponds to the general formula (1), wherein R₁ is

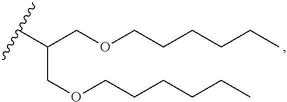

### R₂ is

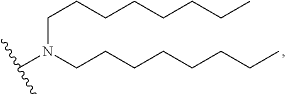 [SMILES: *N(CCCCCCCC)CCCCCCCC]

B₁ and B₂ are both heptylene groups, B₂ is a hexylene group, L₁ is an ester group (—OC(═O)—), L₂ is an ester group (—C(═O)O—), X is N, L₃ is a butylene group, R₃ is a hydroxyl group, and the total process is represented as follows:

The preparation process is represented as follows:

Step a: Under argon atmosphere, glycerol containing a TBS protected hydroxyl group (S12-1, 3.09 g, 15.0 mmol), K₂CO₃ (6.21 g, 45.0 mmol), and bromohexane (S12-2, 2.71 g, 16.5 mmol) were dissolved in 100 mL DMF, the mixture was stirred at 110° C. for 16h. After confirming the completion of the reaction through thin layer chromatography, the reaction solution was poured into water for precipitation, the precipitate was filtered, separated and purified by column chromatography to obtain the glycerol etherification compound with a TBS protected hydroxyl group S12-3 (3.35 g, 89.3%).

Step b: Into a flask under nitrogen protection, the above product S12-3 (1.88 g, 5.0 mmol) was dissolved in THF (50 mL), tetrabutylammonium fluoride solution (TBAF, 50 mL, 1M) was added, and the reaction was conducted overnight to remove the TBS protecting group. The reaction solution was dried with anhydrous sodium sulfate, filtered, and concentrated to obtain the crude product of S12-4. The crude product was purified by column chromatography, concentrated, and dried with an oil pump to obtain the glycerol etherification compound S12-4 (1.14 g, 87.9%).

Step c: Under argon atmosphere, into the round-bottom flask containing S12-4 (0.62 g, 2.4 mmol), 8-bromooctanoic acid (S12-5, 0.45 g, 2.0 mmol) and DMAP (61.00 mg, 0.5 mmol) dissolved in dichloromethane (50 mL), DCC (0.91 g, 4.4 mmol) was added and the reaction was conducted for 16 h at room temperature. After completion of the reaction, the precipitate was removed by filtering. The filtrate was concentrated and purified by silica gel column chromatography to obtain the bromoester S12-6 (0.76 g).

Step d: Under nitrogen protection, the compound S1-6 (0.09 g, 1.0 mmol) was dissolved in acetonitrile (20 mL), S12-6 (0.58 g, 1.3 mmol) and DIPEA (0.09 g, 1.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S12-7 (0.39 g).

Step e: Under nitrogen protection, the compound S12-7 (0.28 g, 0.6 mmol) was dissolved in acetonitrile (20 mL), S4-2 (0.37 g, 0.8 mmol) and DIPEA (0.05 g, 0.6 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E12-1 (0.42 g). The main data of ¹H-NMR spectrum of E12-1 were as follows: ¹H NMR (400 MHz, CDCl₃) δ: 5.15-5.07 (m, 1H), 4.03 (t, 2H), 3.70 (t, 2H), 3.58-3.50 (m, 4H), 3.48-3.36 (m, 4H), 3.22-2.91 (m, 10H), 2.35-2.28 (m, 2H), 1.96-1.47 (m, 20H), 1.38-1.23 (m, 44H), 0.87 (t, 12H). The molecular weight of E12-1 was determined to be 854.58 Da by MALDI-TOF.

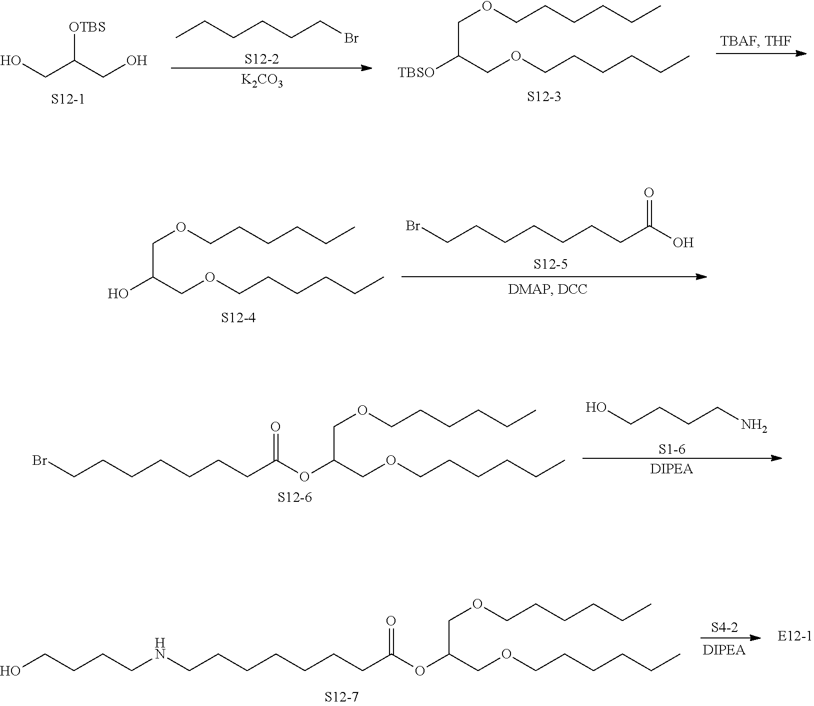

### Example-13: Cationic Lipid (E13-1)

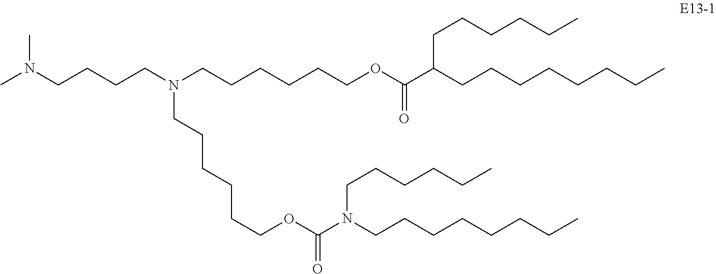 [SMILES: CCCCCCCCC(CCCCCC)C(=O)OCCCCCCN(CCCCCCOC(=O)N(CCCCCC)CCCCCCCC)CCCCN(C)C]

E13-1 corresponds to the general formula (1), wherein R₁ is

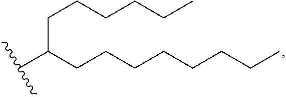

### R₂ is

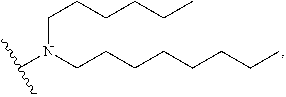 [SMILES: *N(CCCCCC)CCCCCCCC]

B₁ and B₂ are both hexylene groups, L₁ and L₂ are both ester groups (—C(═O)O—), X is N, L₃ is a butylene group, R₃ is

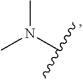 [SMILES: *N(C)C]

and the total molecular weight is approximately 794 Da.

The preparation process is represented as follows:

Step a: Under nitrogen protection, 4-dimethylaminobutylamine (S13-1, 0.09 g, 1.0 mmol) was dissolved in acetonitrile (50 mL), S5-2 (2.10 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S13-2 (1.51 g).

Step b: Under nitrogen protection, the compound S13-2 (0.91 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E13-1 (1.28 g). The main data of ¹H-NMR spectrum of E13-1 were as follows: ¹H NMR (400 MHz, CDCl₃) δ: 4.07-4.01 (m, 4H), 3.22-3.08 (m, 4H), 2.92-2.76 (m, 8H), 2.33-2.26 (m, 1H), 2.23 (s, 6H), 1.83-1.22 (m, 64H), 0.87 (t, 12H). The molecular weight of E13-1 was determined to be 793.74 Da by MALDI-TOF.

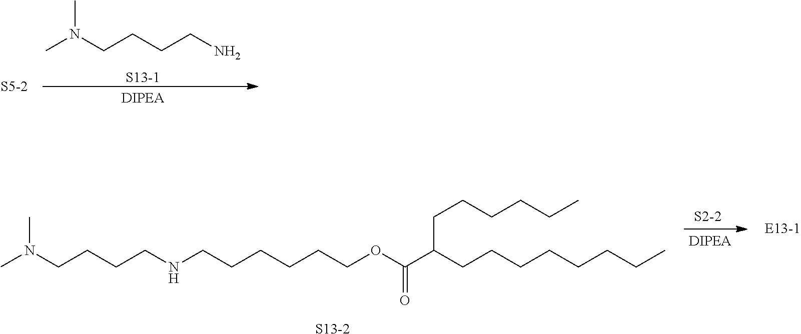

### Example-14: Cationic Lipid (E14-1)

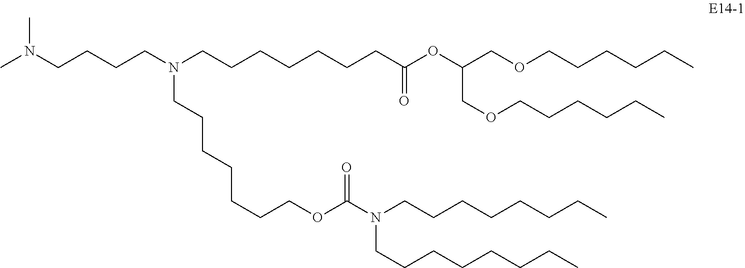 [SMILES: CCCCCCCCN(CCCCCCCC)C(=O)OCCCCCCCN(CCCCCCCC(=O)OC(COCCCCCC)COCCCCCC)CCCCN(C)C]

E14-1 corresponds to the general formula (1), wherein R₁ is

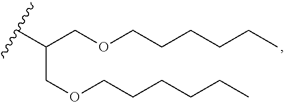

### R₂ is

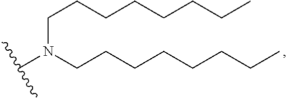 [SMILES: *N(CCCCCCCC)CCCCCCCC]

B₁ and B₂ are both heptylene groups, L₁ and L₂ are both ester groups (—C(═O)O—), X is N, L₃ is a butylene group, R₃ is

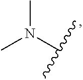 [SMILES: *N(C)C]

and the total molecular weight is approximately 822 Da.

The preparation process is represented as follows:

Referring to the preparation process of E13-1, the same molar equivalent of S13-1, S12-6 and S4-2 as in Example-13 were used as starting materials to obtain the cationic lipid E14-1 (1.43 g). The main data of ¹H-NMR spectrum of E14-1 were as follows: ¹H NMR (400 MHz, CDCl₃) δ: 5.12-5.08 (m, 1H), 4.04 (t, 2H), 3.58-3.52 (m, 4H), 3.48-3.36 (m, 4H), 3.23-2.92 (m, 12H), 2.32-2.28 (m, 2H), 2.23 (s, 6H), 1.96-1.22 (m, 64H), 0.87 (t, 12H). The molecular weight of E14-1 was determined to be 881.82 Da by MALDI-TOF.

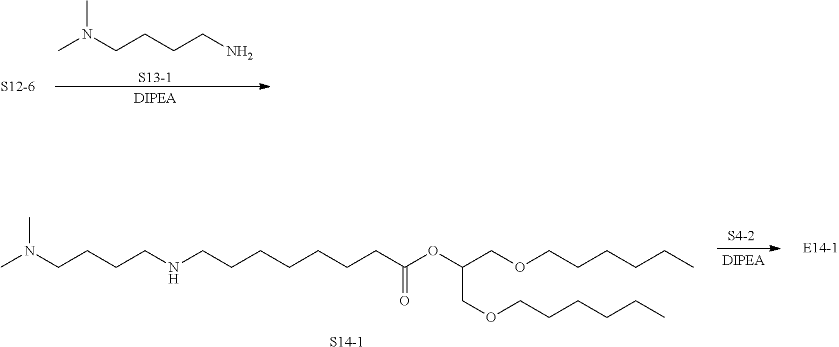

### Example-15: Cationic Lipid (E15-1)

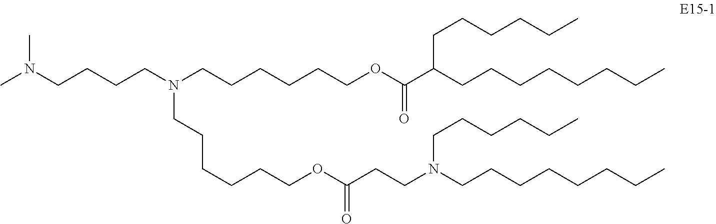 [SMILES: CCCCCCCCC(CCCCCC)C(=O)OCCCCCCN(CCCCCCOC(=O)CCN(CCCCCC)CCCCCCCC)CCCCN(C)C]

E15-1 corresponds to the general formula (1), wherein R₁ is

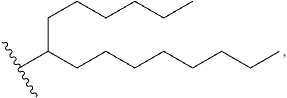

### R₂ is

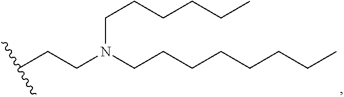 [SMILES: *CCN(CCCCCC)CCCCCCCC]

B₁ and B₂ are both hexylene groups, L₁ is an ester group (—OC(═O—), L₂ is an ester group (—C(═O)O—), X is N, L₃ is a butylene group, R₃ is

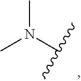 [SMILES: *N(C)C]

and the total molecular weight is approximately 882 Da.

The preparation process is represented as follows:

Referring to the preparation process of E13-1, cationic lipid E15-1 (1.33 g) was obtained by using the S13-1, S5-2 and S1-5 as the raw material, with the same molar equivalent in Example-13. The main data of ¹H-NMR spectrum of E15-1 were as follows: ¹H NMR (400 MHz, CDCl₃) δ: 4.06 (t, 2H), 4.01 (t, 2H), 3.63 (t, 2H), 3.20 (t, 2H), 3.02-2.81 (m, 12H), 2.26 (t, 1H), 2.20 (s, 6H), 1.92-1.21 (m, 64H), 0.83 (t, 12H). The molecular weight of E15-1 was determined to be 821.77 Da by MALDI-TOF.

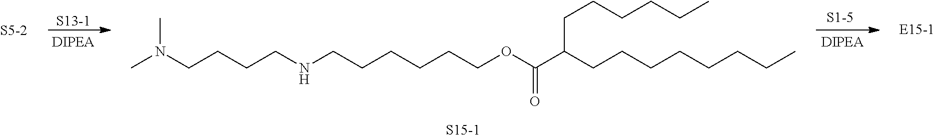 [SMILES: "S13-1" ; "S5-2" ; CCCCCCCCC(CCCCCC)C(=O)OCCCCCCNCCCCN(C)C ; "S1-5" ; "E15-1" ; "S15-1"]

### Example-16: Cationic Lipid (E16-1)

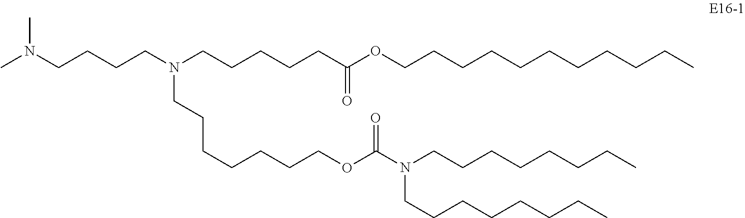 [SMILES: CCCCCCCCCCCOC(=O)CCCCCN(CCCCCCCOC(=O)N(CCCCCCCC)CCCCCCCC)CCCCN(C)C]

E16-1 corresponds to the general formula (1), wherein R₁ is an undecyl group, R₂ is

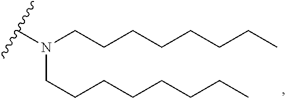

B₁ is a pentylene group, B₂ is a heptylene group, L₁ is an ester group (—OC(═O)—), L₂ is an ester group (—C(═O)O—), X is N, L₃ is a butylene group, R₃ is

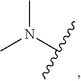 [SMILES: *N(C)C]

and the total molecular weight is approximately 766 Da.

The preparation process is represented as follows:

Step a: Compound S3-1 (2.89 g, 12.0 mmol) was dissolved in dichloromethane (60 mL), and then 7-bromoheptyl-N-succinimide carbonate (S16-1, 3.35 g, 10.0 mmol) and TEA (1.10 mL, 15.0 mmol) were added successively, and the reaction was stirred at room temperature overnight. After completion of the reaction, the reaction solution was concentrated to obtain the crude product. The crude product was purified by column chromatography, concentrated, and dried with an oil pump to obtain the bromoester S16-2 (3.65 g).

Step b: Under nitrogen protection, the compound S13-1 (0.46 g, 4.0 mmol) was dissolved in acetonitrile (50 mL), S16-2 (2.31 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid S16-3 (1.62 g).

Step c: Under nitrogen protection, the S1-6 (1.00 g, 2.0 mmol) was dissolved in acetonitrile (30 mL), undecyl 6-bromohexanoate (S16-4, 0.87 g, 2.5 mmol, wherein S16-4 is a product of reaction between 6-bromohexanoic acid and undecanol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E16-1 (1.26 g). The main data of ¹H-NMR spectrum of E16-1 were as follows: ¹H NMR (400 MHz, CDCl₃) δ: 4.03 (t, 4H), 3.22-3.09 (m, 4H), 2.90-2.79 (m, 8H), 2.30 (t, 2H), 2.23 (s, 6H), 1.76-1.19 (m, 62H), 0.87 (t, 9H). The molecular weight of E16-1 was determined to be 765.74 Da by MALDI-TOF.

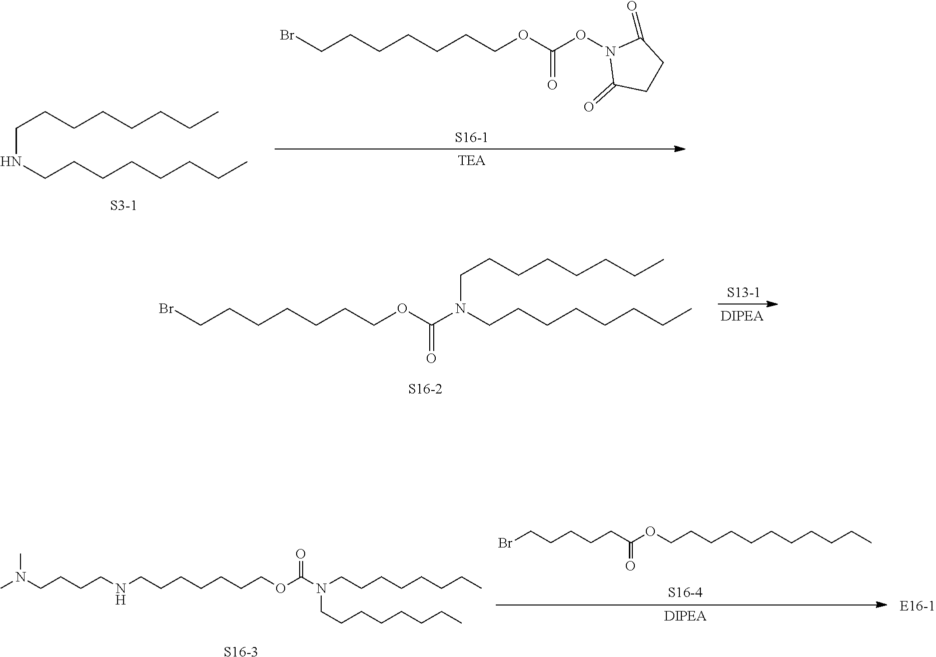

### Example-17: Cationic Lipid (E17-1)

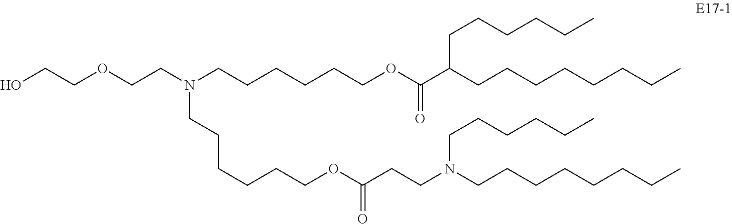 [SMILES: CCCCCCCCC(CCCCCC)C(=O)OCCCCCCN(CCCCCCOC(=O)CCN(CCCCCC)CCCCCCCC)CCOCCO]

E17-1 corresponds to the general formula (1), wherein R₁ is

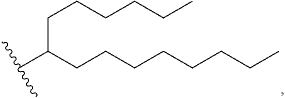

### R₂ is

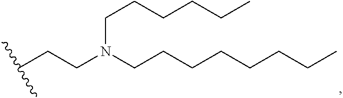 [SMILES: *CCN(CCCCCC)CCCCCCCC]

B₁ and B₂ are both hexylene groups, L₁ and L₂ are both ester groups (—C(═O)O—), X is N, L₃ is —CH₂CH₂OCH₂CH₂—, R₃ is a hydroxyl group, and the total molecular weight is approximately 811 Da.

The preparation process is represented as follows:

Step a: Under nitrogen protection, diglycolamine (S17-1, 0.42 g, 4.0 mmol) was dissolved in acetonitrile (50 mL), S5-2 (2.10 g, 5.0 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S16-3 (1.44 g).

Step b: Under nitrogen protection, the compound S16-3 (0.89 g, 2.0 mmol) was dissolved in acetonitrile (30 mL), S1-5 (0.87 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E17-1 (1.33 g). The main data of ¹H-NMR spectrum of E17-1 were as follows: ¹H NMR (400 MHz, CDCl₃) δ: 4.06 (t, 2H), 4.00 (t, 2H), 3.70 (t, 2H), 3.65-3.63 (m, 6H), 3.20 (t, 2H), 3.02-2.81 (m, 8H), 2.65 (t, 2H), 2.25 (t, 1H), 1.80-1.19 (m, 60H), 0.83 (t, 12H). The molecular weight of E17-1 was determined to be 810.74 Da by MALDI-TOF.

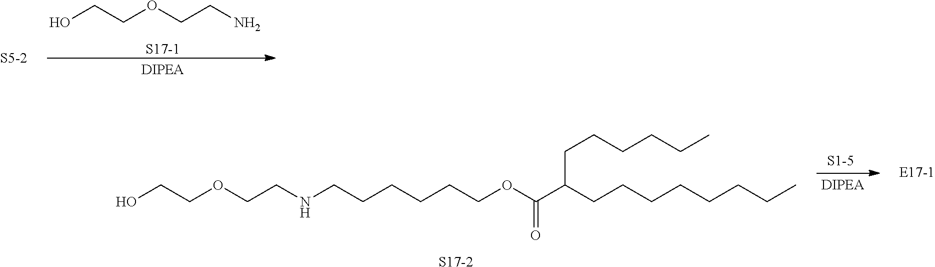

### Example-18: Cationic Lipid (E18-1)

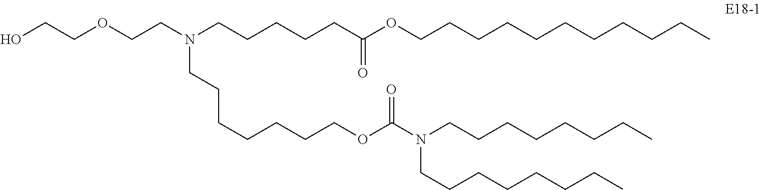 [SMILES: CCCCCCCCCCCOC(=O)CCCCCN(CCCCCCCOC(=O)N(CCCCCCCC)CCCCCCCC)CCOCCO]

E18-1 corresponds to the general formula (1), wherein R₁ is an undecyl group, R₂ is

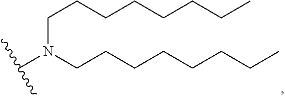

B₁ is a pentylene group, B₂ is a butylene group, L₁ is an ester group (—OC(═O—), L₂ is an ester group (—C(═O)O—), X is N, L₃ is —CH₂CH₂OCH₂CH₂—, R₃ is a hydroxyl group, and the total molecular weight is approximately 755 Da.

The preparation process is represented as follows:

Referring to the preparation process of E13-1, cationic lipid E18-1 (1.24 g) was obtained by using the S16-2, S17-1 and S16-4 as starting materials, with the same molar equivalent in Example-13. The main data of ¹H-NMR spectrum of E18-1 were as follows: ¹H NMR (400 MHz, CDCl₃) δ: 4.03 (t, 4H), 3.71 (t, 2H), 3.63 (t, 4H), 3.22-2.81 (m, 8H), 2.65 (t, 2H), 2.30 (t, 2H), 1.77-1.19 (m, 58H), 0.87 (t, 9H). The molecular weight of E18-1 was determined to be 754.64 Da by MALDI-TOF.

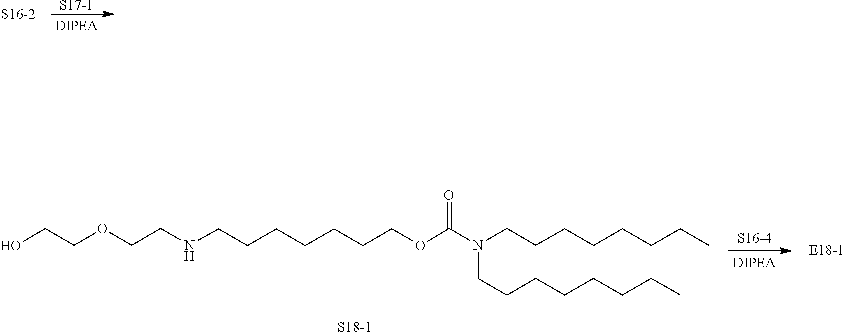

### Example-19: Cationic Lipid (E19-1)

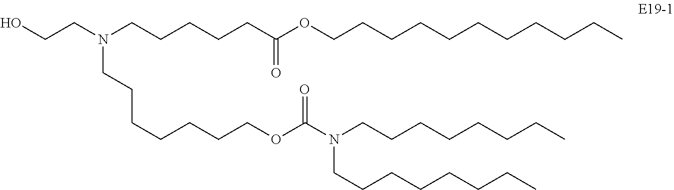 [SMILES: CCCCCCCCCCCOC(=O)CCCCCN(CCO)CCCCCCCOC(=O)N(CCCCCCCC)CCCCCCCC]

E19-1 corresponds to the general formula (1), wherein R₁ is an undecyl group, R₂ is

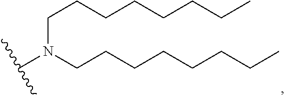

B₁ is a pentylene group, B₂ is a heptylene group, L₁ is an ester group (—OC(═O)—), L₂ is an ester group (—C(═O)O—), X is N, L₃ is an ethylene group, R₃ is a hydroxyl group, and the total molecular weight is approximately 711 Da.

The preparation process is represented as follows:

Referring to the preparation process of E13-1, cationic lipid E19-1 (1.15 g) was obtained by using the S16-2, S17-1 and S16-4 as starting materials, with the same molar equivalent in Example-13. The main data of ¹H-NMR spectrum of E19-1 were as follows: ¹H NMR (400 MHz, CDCl₃) δ: 4.03 (t, 4H), 3.86-3.78 (m, 2H), 3.22-3.09 (m, 4H), 2.98-2.81 (m, 6H), 2.30 (t, 2H), 1.79-1.20 (m, 58H), 0.88 (t, 9H). The molecular weight of E19-1 was determined to be 710.70 Da by MALDI-TOF.

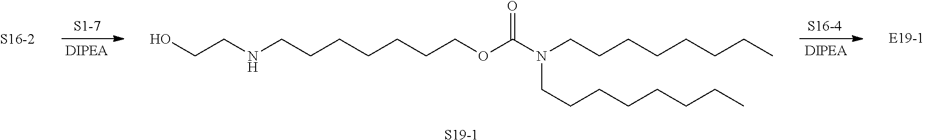 [SMILES: "S16-2" ; "S1-7" ; CCCCCCCCN(CCCCCCCC)C(=O)OCCCCCCCNCCO ; "S16-4" ; "E19-1" ; "S19-1"]

### Example-20: Cationic Lipid (E20-1)

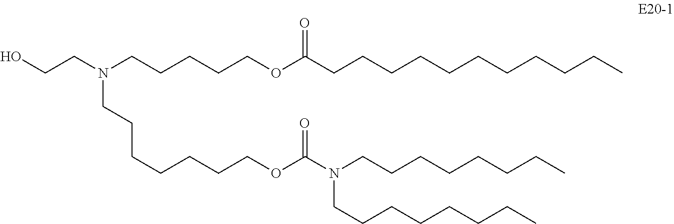 [SMILES: CCCCCCCCCCCC(=O)OCCCCCN(CCO)CCCCCCCOC(=O)N(CCCCCCCC)CCCCCCCC]

E20-1 corresponds to the general formula (1), wherein R₁ is an undecyl group, R₂ is

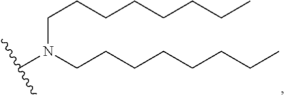

B₁ is a pentylene group, B₂ is a heptylene group, L₁ and L₂ are both ester groups (—C(═O)O—), X is N, L₃ is an ethylene group, R₃ is a hydroxyl group, and the total molecular weight is approximately 711 Da.

The preparation process is represented as follows:

Under nitrogen protection, S16-3 (0.89 g, 2.0 mmol) was dissolved in acetonitrile (50 mL), 5-bromopentyl laurate (S20-1, 0.87 g, 2.5 mmol, wherein S20-1 is a product of reaction between lauric acid and 5-bromo-1-pentanol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound E20-1 (1.11 g). The main data of ¹H-NMR spectrum of E20-1 were as follows: ¹H NMR (400 MHz, CDCl₃) δ: 4.03 (t, 4H), 3.86-3.78 (m, 2H), 3.22-3.09 (m, 4H), 2.98-2.83 (m, 6H), 2.32 (t, 2H), 1.76-1.22 (m, 58H), 0.86 (t, 9H). The molecular weight of E20-1 was determined to be 710.92 Da by MALDI-TOF.

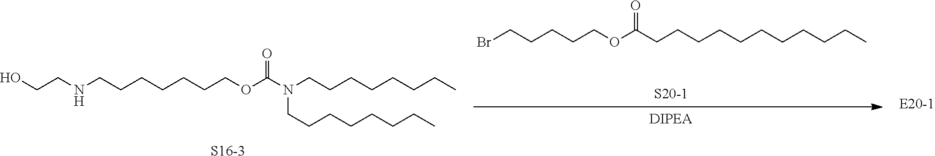 [SMILES: "S16-3" ; CCCCCCCCN(CCCCCCCC)C(=O)OCCCCCCCNCCO ; CCCCCCCCCCCC(=O)OCCCCCBr ; "S20-1" ; "E20-1"]

### Example-21: Cationic Lipid (E21-1)

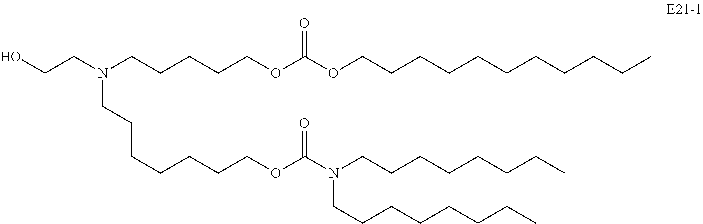 [SMILES: CCCCCCCCCCCOC(=O)OCCCCCN(CCO)CCCCCCCOC(=O)N(CCCCCCCC)CCCCCCCC]

E21-1 corresponds to the general formula (1), wherein R₁ is an undecyl group, R₂ is

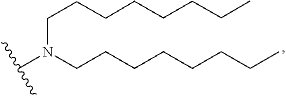

B₁ is a pentylene group, B₂ is a heptylene group, L₁ is a carbonate group (—OC(═O)O—), L₂ is an ester group (—C(═O)O—), X is N, L₃ is an ethylene group, R₃ is a hydroxyl group, and the total molecular weight is approximately 727 Da.

The preparation process is represented as follows:

Step a: Under nitrogen protection, S10-1 (4.14 g, 12.0 mmol) was dissolved in dichloromethane (200 mL), 1-undecanol (S12-1 8.26 g, 48.0 mmol) was added dropwise slowly with stirring at room temperature, pyridine (0.29 mL, 2.4 mmol) was then added dropwise slowly over 10 min, followed by a single addition of DMAP (0.29 g, 2.4 mmol). The reaction was stirred at room temperature for 16 h, after completion of the reaction, the reaction solution was extracted twice with dichloromethane, the organic phases were combined and washed with saline, dried with anhydrous MgSO₄, and filtered to obtain the crude product. The crude product was purified by silica gel column. The target eluent was collected and concentrated to obtain the 6-bromohexadecyl carbonate (S21-2, 1.18 g).

Step b: Under nitrogen protection, the compound S1-7 (0.12 g, 2.0 mmol) was dissolved in acetonitrile (30 mL), S21-2 (1.08 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S21-3 (0.60 g).

Step c: Under nitrogen protection, the compound S21-3 (0.36 g, 1.0 mmol) was dissolved in acetonitrile (20 mL), S16-2 (0.58 g, 1.3 mmol) and DIPEA (0.09 g, 1.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E21-1 (0.59 g). The main data of ¹H-NMR spectrum of E21-1 were as follows: ¹H NMR (400 MHz, CDCl₃) δ: 4.19 (t, 4H), 4.03 (t, 2H), 3.86-3.78 (m, 2H), 3.22-3.09 (m, 4H), 2.96-2.81 (m, 6H), 1.76-1.23 (m, 58H), 0.87 (t, 9H). The molecular weight of E21-1 was determined to be 726.63 Da by MALDI-TOF.

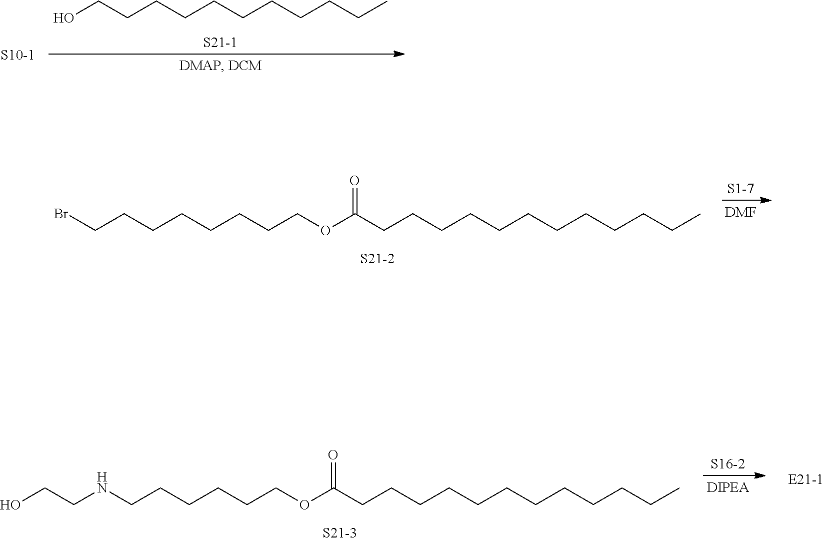

### Example-22: Cationic Lipid (E22-1)

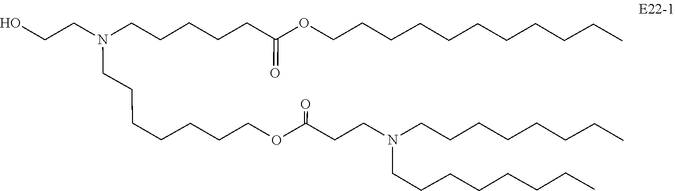 [SMILES: CCCCCCCCCCCOC(=O)CCCCCN(CCO)CCCCCCCOC(=O)CCN(CCCCCCCC)CCCCCCCC]

E22-1 corresponds to the general formula (1), wherein R₁ is an undecyl group, R₂ is

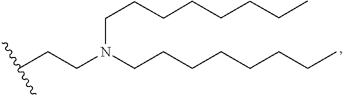

B₁ is a pentylene group, B₂ is a heptylene group, L₁ is an ester group (—OC(═O—), L₂ is an ester group (—C(═O)O—), X is N, L₃ is an ethylene group, R₃ is a hydroxyl group, and the total molecular weight is approximately 739 Da.

The preparation process is represented as follows:

Step a: Under nitrogen protection, the compound S1-7 (0.12 g, 2.0 mmol) was dissolved in acetonitrile (30 mL), S22-1 (1.23 g, 2.5 mmol, wherein S22-1 is a product of a reaction between 7-bromo-1-heptanol and

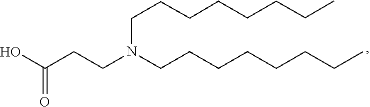 [SMILES: CCCCCCCCN(CCCCCCCC)CCC(=O)O]

referring to Step b in Example-1.1 for specific experimental steps) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid S22-2 (0.78 g).

Step b: Under nitrogen protection, the S22-2 (0.47 g, 1.0 mmol) was dissolved in acetonitrile (20 mL), S16-4 (0.44 g, 1.3 mmol) and DIPEA (0.09 g, 1.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E22-1 (0.59 g). The main data of ¹H-NMR spectrum of E22-2 were as follows: ¹H NMR (400 MHz, CDCl₃) δ: 4.03 (t, 4H), 3.86-3.78 (m, 2H), 3.63 (t, 2H), 3.22-3.09 (m, 4H), 2.99-2.81 (m, 6H), 2.30 (t, 4H), 1.81-1.19 (m, 58H), 0.88 (t, 9H). The molecular weight of E22-1 was determined to be 738.65 Da by MALDI-TOF.

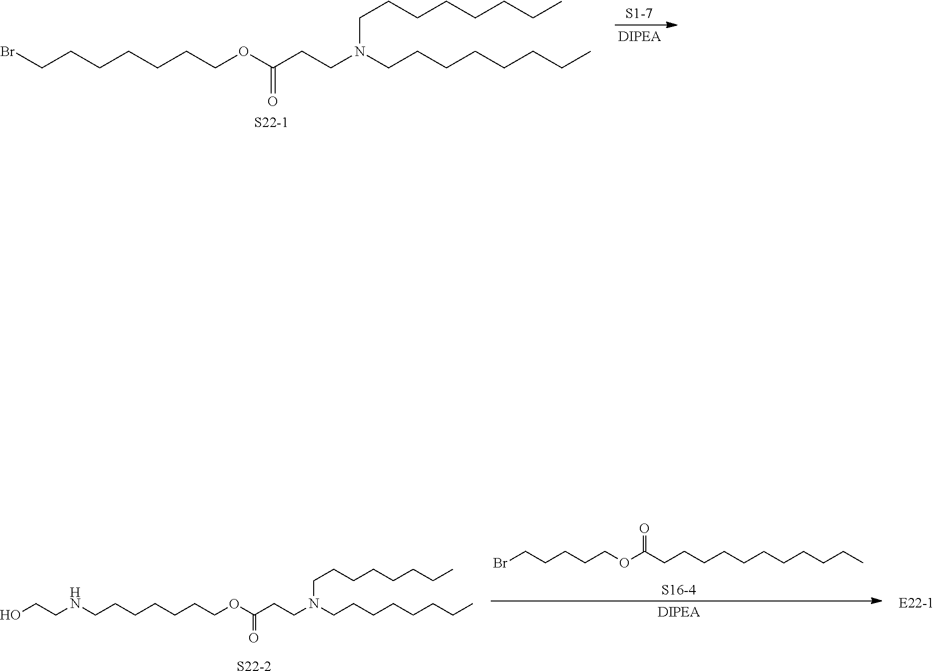

### Example-23: Cationic Lipid (E23-1)

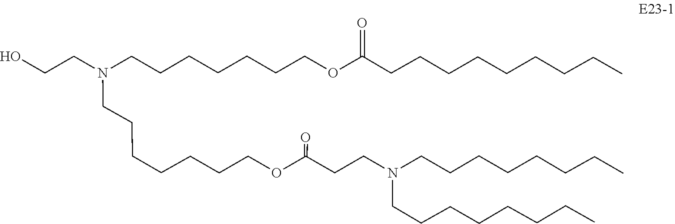 [SMILES: CCCCCCCCCC(=O)OCCCCCCCN(CCO)CCCCCCCOC(=O)CCN(CCCCCCCC)CCCCCCCC]

E23-1 corresponds to the general formula (1), wherein R₁ is a nonyl group, R₂ is

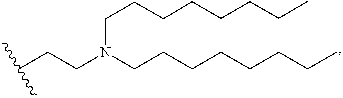

B₁ and B₂ are both heptylene groups, L₁ and L₂ are both ester groups (—C(═O)O—), X is N, L₃ is an ethylene group, R₃ is a hydroxyl group, and the total molecular weight is approximately 739 Da.

The preparation process is represented as follows:

Under nitrogen protection, S22-2 (0.94 g, 2.0 mmol) was dissolved in acetonitrile (20 mL), S23-1 (0.87 g, 2.5 mmol, wherein S23-1 is a product of reaction between 7-bromo-1-heptanol and decanoic acid, referring to Step b in Example-1.1 for specific experimental steps) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound E23-1 (1.21 g). The main data of ¹H-NMR spectrum of E23-1 were as follows: ¹H NMR (400 MHz, CDCl₃) δ: 4.03 (t, 4H), 3.86-3.76 (m, 2H), 3.64 (t, 2H), 3.22-3.09 (m, 4H), 2.98-2.81 (m, 6H), 2.31 (t, 4H), 1.75-1.21 (m, 58H), 0.87 (t, 9H). The molecular weight of E23-1 was determined to be 738.69 Da by MALDI-TOF.

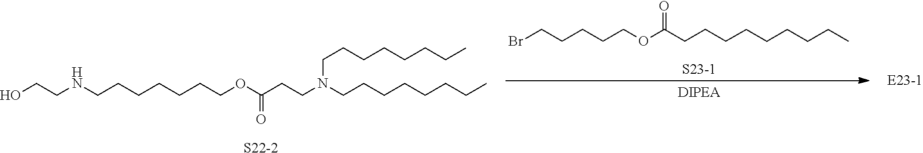

### Example-24: Cationic Lipid (E24-1)

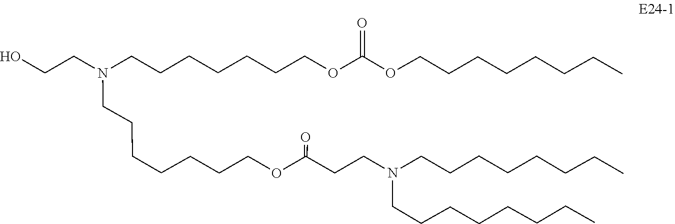 [SMILES: CCCCCCCCOC(=O)OCCCCCCCN(CCO)CCCCCCCOC(=O)CCN(CCCCCCCC)CCCCCCCC]

E24-1 corresponds to the general formula (1), wherein R₁ is an octyl group, R₂ is

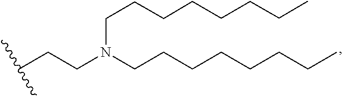

B₁ and B₂ are both heptylene groups, L₁ is a carbonate group (—OC(═O)O—), L₂ is an ester group (—C(═O)O—), X is N, L₃ is an ethylene group, R₃ is a hydroxyl group, and the total molecular weight is approximately 741 Da.

The preparation process is represented as follows:

Under nitrogen protection, S22-2 (0.94 g, 2.0 mmol) was dissolved in acetonitrile (20 mL), S24-1 (0.88 g, 2.5 mmol, wherein S24-1 is a product of a reaction between 7-bromoheptyl-4-nitrophenyl carbonate and octanol, referring to Step a in Example-10 for specific experimental steps) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound E24-1 (1.22 g). The main data of ¹H-NMR spectrum of E24-1 were as follows: ¹H NMR (400 MHz, CDCl₃) δ: 4.19 (t, 4H), 4.03 (t, 2H), 3.85-3.78 (m, 2H), 3.63 (t, 2H), 3.22-3.09 (m, 4H), 2.98-2.83 (m, 6H), 2.30 (t, 2H), 1.79-1.19 (m, 56H), 0.87 (t, 9H). The molecular weight of E24-1 was determined to be 740.68 Da by MALDI-TOF.

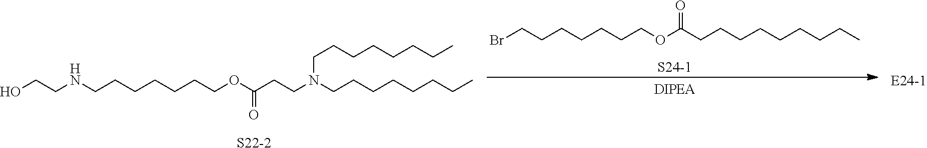

### Example-25: Cationic Lipid (E25-1)

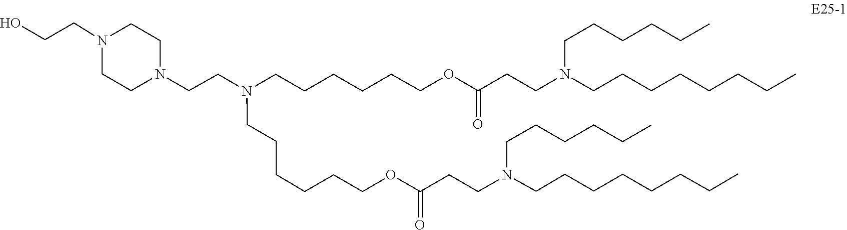 [SMILES: CCCCCCCCN(CCCCCC)CCC(=O)OCCCCCCN(CCCCCCOC(=O)CCN(CCCCCC)CCCCCCCC)CCN1CCN(CCO)CC1]

E25-1 corresponds to the general formula (1), wherein R₁ and R₂ are both

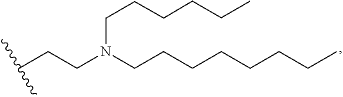

B₁ and B₂ are both hexylene groups, L₁ and L₂ are both ester groups (—C(═O)O—), X is N, L₃ is

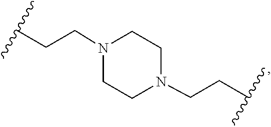 [SMILES: *CCN1CCN(CC*)CC1]

is a hydroxyl group, and the total molecular weight is approximately 908 Da.

The preparation process is represented as follows:

Under nitrogen protection, 2-(4-(2-aminoethyl) piperazin-1-yl) ethanol (S25-2, 0.35 g, 2.0 mmol) was dissolved in acetonitrile (100 mL), S1-5 (2.24 g, 5.0 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound E25-1 (1.48 g). The main data of ¹H-NMR spectrum of E25-1 were as follows: ¹H NMR (400 MHz, CDCl₃) δ: 4.03 (t, 4H), 3.71 (t, 2H), 3.63 (t, 4H), 3.12-2.49 (m, 26H), 2.31 (t, 4H), 1.78-1.19 (m, 56H), 0.87 (t, 12H). The molecular weight of E25-1 was determined to be 907.83 Da by MALDI-TOF.

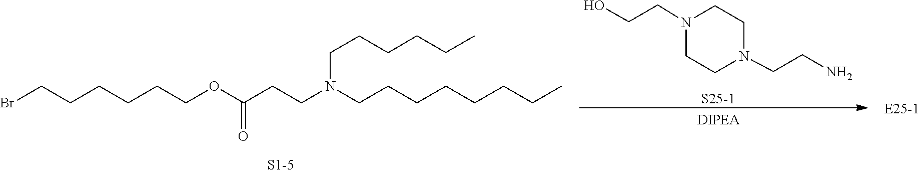

### Example-26: Cationic Lipid (E26-1)

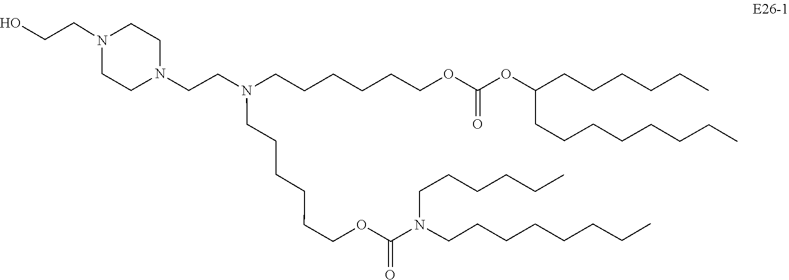 [SMILES: CCCCCCCCC(CCCCCC)OC(=O)OCCCCCCN(CCCCCCOC(=O)N(CCCCCC)CCCCCCCC)CCN1CCN(CCO)CC1]

E26-1 corresponds to the general formula (1), wherein R₁ is

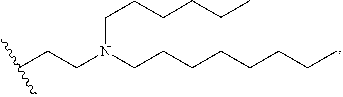

### R₂ is

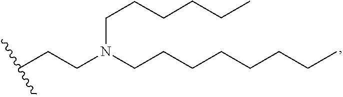 [SMILES: *CCN(CCCCCC)CCCCCCCC]

B₁ and B₂ are both hexylene groups L₁ is a carbonate group (—OC(═O)O—), L₂ is an ester group (—C(═O)O—), X is N, L₃ is

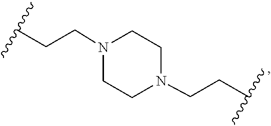 [SMILES: *CCN1CCN(CC*)CC1]

R₃ is a hydroxyl group, and the total molecular weight is approximately 867 Da.

The preparation process is represented as follows:

Step a: Under nitrogen protection, the compound S25-1 (0.69 g, 4.0 mmol) was dissolved in acetonitrile (50 mL), S10-2 (2.17 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid S26-1 (1.67 g).

Step b: Under nitrogen protection, the S26-1 (1.06 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E26-1 (1.39 g). The main data of ¹H-NMR spectrum of E26-1 were as follows: ¹H NMR (400 MHz, CDCl₃) δ: 4.71-4.68 (m, 1H), 4.21 (t, 2H), 4.03 (t, 2H), 3.71 (t, 2H), 3.12-2.49 (m, 18H), 2.55-2.46 (m, 4H), 1.75-1.25 (m, 60H), 0.89 (t, 12H). The molecular weight of E26-1 was determined to be 866.75 Da by MALDI-TOF.

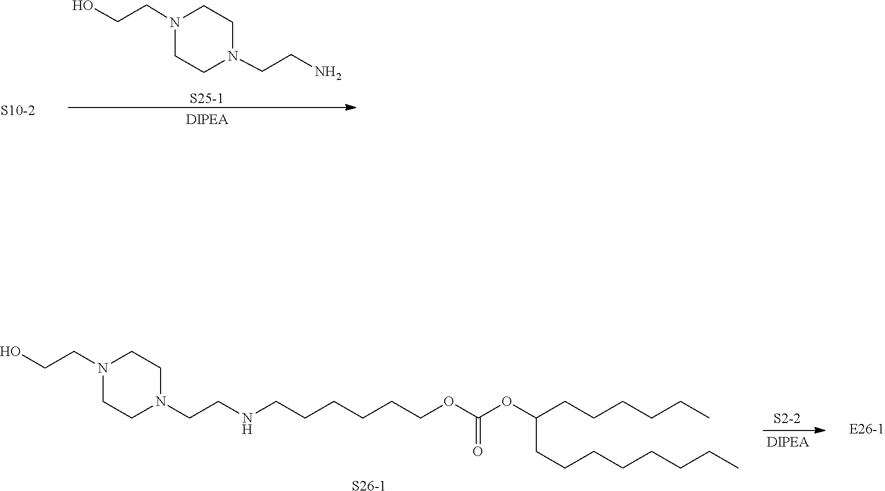

### Example-27: Cationic Lipid (E27-1)

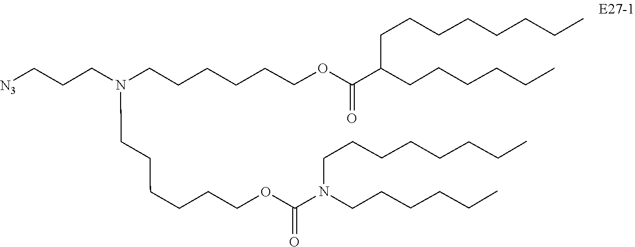 [SMILES: CCCCCCCCC(CCCCCC)C(=O)OCCCCCCN(CCCCCCOC(=O)N(CCCCCC)CCCCCCCC)CCCN=[N+]=[N-]]

E27-1 corresponds to the general formula (1), wherein R₁ is

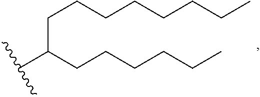

### R₂ is

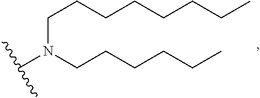 [SMILES: *N(CCCCCC)CCCCCCCC]

B₁ and B₂ are both hexylene groups, L₁ and L₂ are both ester groups (—C(═O)O—), X is N, L₃ is a propylene group, R₃ is an azido group, and the total molecular weight is approximately 778 Da.

The preparation process is represented as follows:

Step a: Under nitrogen protection, 3-azidopropylamine (S27-1, 0.40 g, 4.0 mmol) was dissolved in acetonitrile (50 mL), S5-2 (2.09 g, 5.0 mmol) and DIPEA (0.36 g, 4.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the compound S27-2 (1.41 g).

Step b: Under nitrogen protection, the compound S27-2 (0.88 g, 2.0 mmol) was dissolved in acetonitrile (30 mL), S2-2 (1.05 g, 2.5 mmol) and DIPEA (0.18 g, 2.0 mmol) were added successively with stirring slowly, and the reaction was stirred at room temperature for about 20 h. After completion of the reaction, the reaction solution was concentrated, dissolved in dichloromethane, and extracted with 0.6M HCl/10% NaCl solution and saturated sodium bicarbonate solution successively. The organic phases were combined, dried with anhydrous MgSO₄, filtered, concentrated, and purified by column chromatography to obtain the cationic lipid E27-1 (1.23 g). The main data of ¹H-NMR spectrum of E27-1 were as follows: ¹H NMR (400 MHz, CDCl₃) δ: 4.06 (t, 2H), 4.01 (t, 2H), 3.24-3.06 (m, 4H), 2.90-2.59 (m, 6H), 2.25 (t, 1H), 1.81-1.22 (m, 64H), 0.85 (t, 12H). The molecular weight of E27-1 was determined to be 777.72 Da by MALDI-TOF.

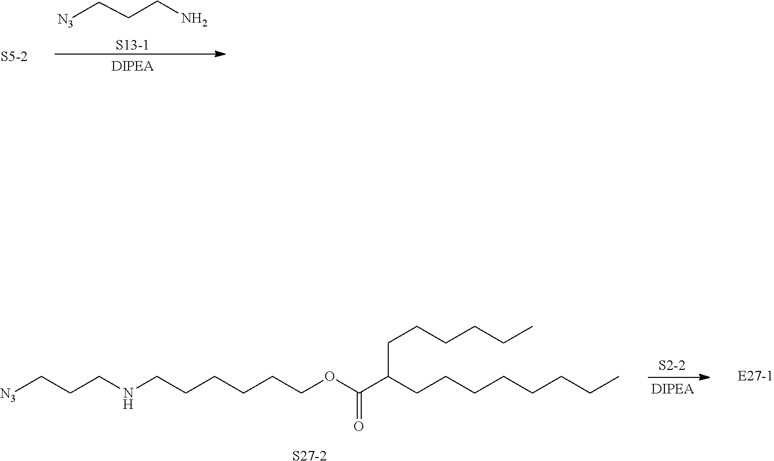

### Example-28: Preparation of the Formulations of Cationic Liposome-Nucleic Acid Pharmaceutical Compositions

TABLE 1
Formulation of each liposome and physicochemical properties
of liposome pharmaceutical compositions

| Liposome | L-CT1 | L-CT2 | L-1 | L-2 | L-3 | L-4 |
|---|---|---|---|---|---|---|
| Cationic lipid CL | ALC-0315 | SM102 | E1-1 | E1-2 | E1-3 | E2-1 |
| Particle size (nm) | 105 | 109 | 103 | 106 | 107 | 106 |
| Encapsulation | 92.0 | 91.6 | 85.3 | 83.2 | 82.4 | 91.3 |
| efficiency (%) | | | | | | |
| Liposome | L-5 | L-6 | L-7 | L-8 | L-9 | L-10 |
| Cationic lipid CL | E3-1 | E4-1 | E5-1 | E6-1 | E6-2 | E7-1 |
| Particle size (nm) | 102 | 103 | 105 | 108 | 106 | 105 |
| Encapsulation | 90.8 | 90.9 | 88.7 | 93.6 | 93.3 | 92.8 |
| efficiency (%) | | | | | | |
| Liposome | L-11 | L-12 | L-13 | L-14 | L-15 | L-16 |
| Cationic lipid CL | E7-2 | E8-1 | E9-1 | E10-1 | E11-1 | E12-1 |
| Particle size (nm) | 109 | 98 | 102 | 107 | 103 | 105 |
| Encapsulation | 92.2 | 88.5 | 92.5 | 90.1 | 87.8 | 92.9 |
| efficiency (%) | | | | | | |
| Liposome | L-17 | L-18 | L-19 | L-20 | L-21 | L-22 |
| Cationic lipid CL | E13-1 | E14-1 | E15-1 | E16-1 | E17-1 | E18-1 |
| Particle size (nm) | 112 | 114 | 106 | 105 | 102 | 107 |
| Encapsulation | 92.1 | 92.6 | 87.2 | 92.1 | 89.2 | 91.5 |
| efficiency (%) | | | | | | |
| Liposome | L-23 | L-24 | L-25 | L-26 | L-27 | L-28 |
| Cationic lipid CL | E19-1 | E20-1 | E21-1 | E22-1 | E23-1 | E24-1 |
| Particle size (nm) | 106 | 107 | 106 | 103 | 98 | 102 |
| Encapsulation | 92.5 | 91.1 | 91.3 | 87.4 | 88.1 | 87.5 |
| efficiency (%) | | | | | | |
| Liposome | L-29 | L-30 | L-31 | | | |
| Cationic lipid CL | E25-1 | E26-1 | E27-1 | | | |
| Particle size (nm) | 99 | 105 | 103 | | | |
| Encapsulation | 85.6 | 91.3 | 90.1 | | | |
| efficiency (%) | | | | | | |

In this example, multiple groups of cationic liposomes were prepared for comparison. In the compositions of every group of cationic liposomes, the neutral lipids contained were all DSPC, the sterol lipids contained were all cholesterol, the PEGylated lipids contained were all PEG2k-DMG (DMG for short), and the differences lied in the cationic lipid components. Wherein, in the control group 1, the cationic lipid was ALC-0315 prepared according to the preparation process disclosed in the literature CN108368028A; in the control group 2, the cationic lipid was SM102 prepared according to the preparation process disclosed in the literature CN110520409A; in the experimental groups (L-1 to L-31), the cationic lipids contained were the cationic lipids prepared in the present invention, specifically as shown in Table 1.

The preparation method of cationic liposome-nucleic acid pharmaceutical compositions (LNP-mRNA): cationic lipid, DSPC, cholesterol, and PEGylated lipid listed in the table 1 were dissolved in ethanol at a proper molar ratio to obtain ethanol phase solution; Fluc-mRNA was added into 50 mM citrate buffer solution (pH=4) at the N/P ratio of 6:1 to obtain water phase solution; the aforementioned ethanol phase solution and water phase solution were mixed with a volume ratio of 1:3, washed several times by ultrafiltration with DPBS to remove ethanol and free molecules, and finally filtered through a 0.2 m sterile filter to obtain the cationic liposome-nucleic acid pharmaceutical composition.

### Example-29: Physical and Chemical Property Assays for the Cationic Liposome-Nucleic Acid Pharmaceutical Compositions

Determination of encapsulation efficiency: In the example, the encapsulation efficiency of the cationic liposome was determined using the Quant-it Ribogreen RNA quantification kit, and the results showed that the cationic liposomes of the present invention had a high encapsulation efficiency of the nucleic acid drug (mRNA), which were all in the range of 80-95%, and most of the encapsulation efficiency were in the range of 85%-95%, specifically in Table 1. The results also showed that the encapsulation efficiency of cationic lipids containing multiple nitrogen branchings of the invention was higher or lower than that of the control groups, and the lipid compounds with hydrophobic fat tail chains protruded by tertiary amines as nitrogen branching had lower encapsulation efficiency, for example, the encapsulation efficiency of L-1, L-2, L-3, and L-12 were relatively low, whereas the encapsulation efficiency of the cationic lipids with hydrophobic fat tail chains protruded by amines in the urethane bond as nitrogen branching were higher, and the cationic lipids containing hydrophobic fat tail chains protruded by amines in carbamate bonds as nitrogen branching at one end and hydrophobic tail chains protruded by carbon branching at the other end had a better encapsulation effect, such as L-8, L-9, L-10, and L-16.

Determination of particle size: in this example, the particle size of LNP-mRNA was determined by dynamic light scattering (DLS). The measured size of cationic liposome had a relatively high uniformity, and the PDI values were all smaller than 0.3. The particle sizes of cationic liposomes prepared with the lipid compositions of the present invention were in the range of 90-120 nm, specifically in Table 1.

### Example-30: Biological Activity Assays for the Cationic Liposome-Nucleic Acid Pharmaceutical Compositions

### (1) Study on the Cytotoxicity (Biocompatibility)

The cytotoxicity of the formulation of cationic liposome-nucleic acid pharmaceutical composition of the present invention was examined by the MTT assay. The formulations of cationic liposome-nucleic acid pharmaceutical compositions were dissolved in culture medium to make the required dosage. With 293T cells as a cell model, the cell suspension was inoculated onto a 96-well plate at 100 μL/well and a density of 1×10⁴ cells/well. After the inoculation, the cells were incubated in a cell incubator for 24 h, mRNA was administrated at a dose of 0.2 μg per well, the corresponding volume of fresh culture medium was added to the blank control group, and each group corresponded to 3 replicate wells. After the formulations of compositions were incubated with the 293T cells for 24 h, 20 μL PBS buffer solution containing 5 mg/mL MTT was added to each well. After the MTT was incubated with the cancer cells for 4 h, the mix solution of the culture medium and the buffer solution containing MTT was aspirated, and DMSO was added at 150 μL/well. After shaking evenly, the absorbance was measured by a microplate reader. The result was calculated according to the measured absorbance value, and showed that compared with the blank control group, the cell viability in groups containing the formulations of cationic liposome-nucleic acid pharmaceutical compositions prepared in the present invention were all over 95%, indicating that the PEGylated lipid-modified formulations of cationic liposome-nucleic acid pharmaceutical composition of the present invention had good biocompatibility.

(2) Study on Transfection Rates of mRNA at the Cellular Level

Luciferase bioluminescence was used to examine the transfection rate of mRNA at the cellular level of some cationic liposome pharmaceutical compositions (L-CT1, L-CT2, L-1, L-8, L-9, L-10, L-11, L-12, L-16, L-22, and L-23) prepared in Example-28 of the present invention. The formulations of cationic liposome-nucleic acid pharmaceutical composition were dissolved in culture medium to the required concentration; with 293T cell as the cell model, the cell suspension was inoculated onto a 96-well plate with a black edge and transparent bottom at 100 μL/well and a density of 4×10⁴ cells/well. After the inoculation, the cells were incubated in a cell incubator for 24 h, mRNA was administrated at a dose of 0.2 μg per well, the corresponding volume of fresh culture medium was added to the blank control group, and each group corresponded to 3 replicate wells. After 24 h of transfection, the old culture medium was removed and replaced with new culture medium containing D-fluorescein sodium (1.5 mg/mL), after incubation for 5 min, the bioluminescence was detected with a microplate reader, and the stronger fluorescence indicated more Fluc-mRNA were translocated into the cytoplasm and translated into the corresponding fluorescent proteins. The results were shown in Table 2, wherein the relative fluorescence intensity value was the ratio of the fluorescence intensity value of each group to that of the blank control group. The results showed that the cationic liposome-nucleic acid pharmaceutical compositions prepared in the present invention had excellent transfection effects in vitro compared to the blank group, and most cationic liposome-nucleic acid pharmaceutical compositions had a higher transfection efficiency than that of the control group, which further demonstrated that the more tertiary amines in the cationic lipids were not the better, that is, being able to ionize more positive charges was not necessary to present a more excellent encapsulation and transfection efficiency, and the position of ionizable tertiary amine structure was particularly important for the overall performance of cationic lipids. Cationic lipids with the tertiary amine at the polar head of the short chain (e.g., L-8, L-9, L-10, L-11, L-16, L-23) rather than at the hydrophobic long tail chain (e.g., L-1, L-12, L-26) were more conducive to the formation of cationic liposomes, which were able to encapsulate the nucleic acid drug better and facilitated the release of nucleic acid drugs from the endosome to the cytoplasm to produce an effect, thus exhibiting higher encapsulation and cell transfection rates.

TABLE 2
Relative fluorescence values of cell transfection

| Group | control | L-CT1 | L-CT2 | L-1 | L-8 | L-9 | L-10 | L-11 | L-12 |
|---|---|---|---|---|---|---|---|---|---|
| Relative | 1 | 2.8 | 3.0 | 1.7 | 4.2 | 3.7 | 3.6 | 3.4 | 2.3 |
| fluorescence | | | | | | | | | |
| intensity | | | | | | | | | |

| Group | L-13 | L-16 | L-17 | L-18 | L-20 | L-22 | L-23 | L-26 | L-30 |
|---|---|---|---|---|---|---|---|---|---|
| Relative | 3.6 | 3.8 | 3.5 | 3.5 | 3.3 | 3.5 | 3.7 | 2.1 | 2.9 |
| fluorescence | | | | | | | | | |
| intensity | | | | | | | | | |

Those described above are only embodiments of the present invention and are not for the purpose of limitation. Any modification of equivalent structures or equivalent routes according to the present invention, which may be applied in other related art in a direct or an indirect way, should be included in the scope of the present invention. For those skilled in the art, without departing from the spirit and scope of the present invention, and without unnecessary experimentation, the present invention can be implemented in a wide range under equivalent parameters, concentrations, and conditions. While the present invention has given particular examples, it should be understood that the present invention can be further modified. In conclusion, in accordance with the principles of the present invention, the present application is intended to cover any alterations, uses, or improvements of the present invention, including changes made using conventional techniques known in the art, departing from the scope disclosed in this application.

1. A cationic lipid, wherein the structure is represented by the general formula (1): 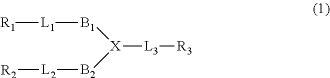
wherein, X is N;
L₁ and L₂ are each independently selected from the group consisting of a linking bond, —OC(═O)—, —C(═O)O—, —OC(═O)O—, —C(═O)—, —O—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—; wherein, R_{c} is, at each occurrence, independently a hydrogen atom or a C₁₋₁₂ alkyl group, s is 2, 3 or 4;
L₃ is a linking bond or a divalent linking group;
B₁ and B₂ are each independently a linking bond or a C₁₋₃₀ alkylene group;
R₁ and R₂ are each independently represented by 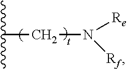
a C₁₋₃₀ aliphatic hydrocarbon group or a residue of C₁₋₃₀ aliphatic hydrocarbon derivative, and at least one of R₁ and R₂ is represented by 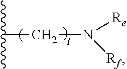
wherein t is an integer from 0 to 12, Rₑ and R_{f} are each independently selected from the group consisting of a C₁₋₁₅ alkyl group, a C₂₋₁₅ alkenyl group, and a C₂₋₁₅ alkynyl group;
R₃ is a hydrogen atom, —R_{d}, —OR_{d}, —NR_{d}R_{d}, —SR_{d}, —C(═O)R_{d}, —C(═O)OR_{d}, —OC(═O)R_{d}, —OC(═O)OR_{d}, or 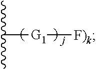
wherein, R_{d} is, at each occurrence, independently a C₁₋₁₂ alkyl group, and two R_{d} in NR_{d}R_{d} can be joined to form a ring; G₁ is a terminal branching group with the valence of k+1; j is 0 or 1; F contains a functional group R₀₁; when j is 0, G₁ is absent, and k is 1; when j is 1, G₁ protrudes F with the number of k, and k is an integer from 2 to 8;
said alkyl group, alkylene group, aliphatic hydrocarbon group, alkenyl group, and alkynyl group are each independently substituted or unsubstituted.

2. The cationic lipid according to claim 1, wherein said C₁₋₃₀ aliphatic hydrocarbon group is a linear alkyl group, a branched alkyl group, a linear alkenyl group, a branched alkenyl group, a linear alkynyl group, or a branched alkynyl group; when said C₁₋₃₀ aliphatic hydrocarbon group is a branched alkyl group, a branched alkenyl group, or a branched alkynyl group, it is represented by 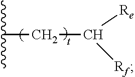
the structure of said residue of C₁₋₃₀ aliphatic hydrocarbon derivative is represented by 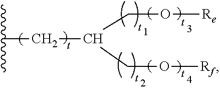
wherein t is an integer from 0 to 12, t₁ and t₂ are each independently an integer from 0 to 5, t₃ and t₄ are each independently 0 or 1, not being 0 simultaneously; wherein, Rₑ and R_{f} are each independently a C₁₋₁₅ alkyl group, a C₂₋₁₅ alkenyl group, or a C₂₋₁₅ alkynyl group;
said C₁₋₃₀ aliphatic hydrocarbon group or residue of C₁₋₃₀ aliphatic hydrocarbon derivative is specifically selected from the following structures:

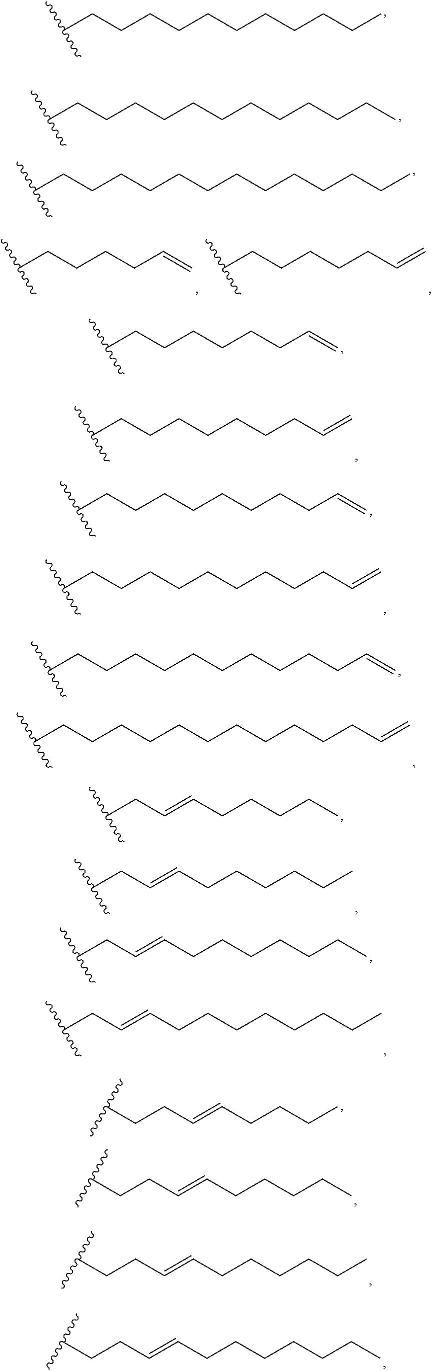

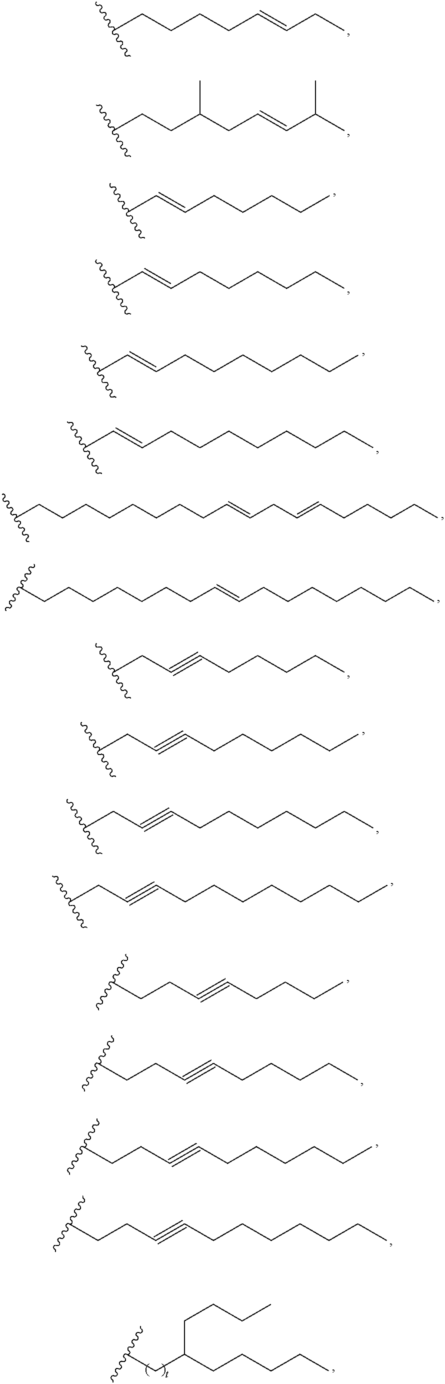

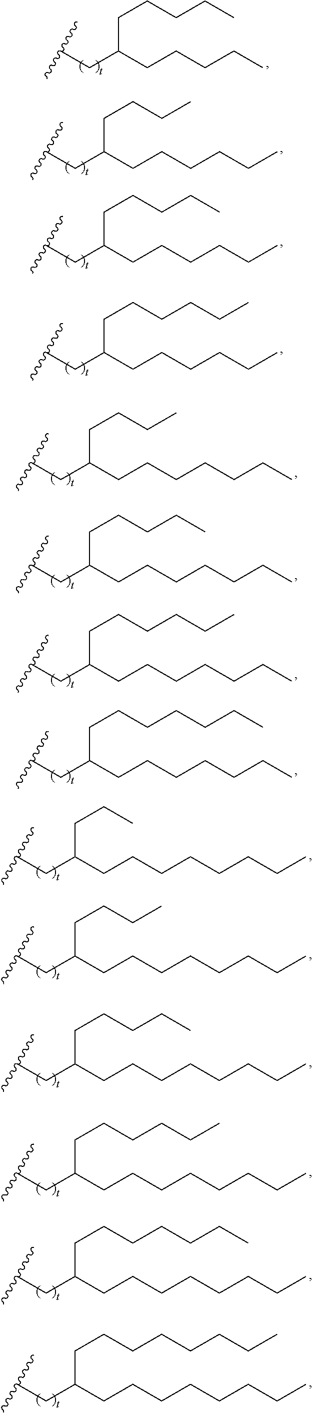

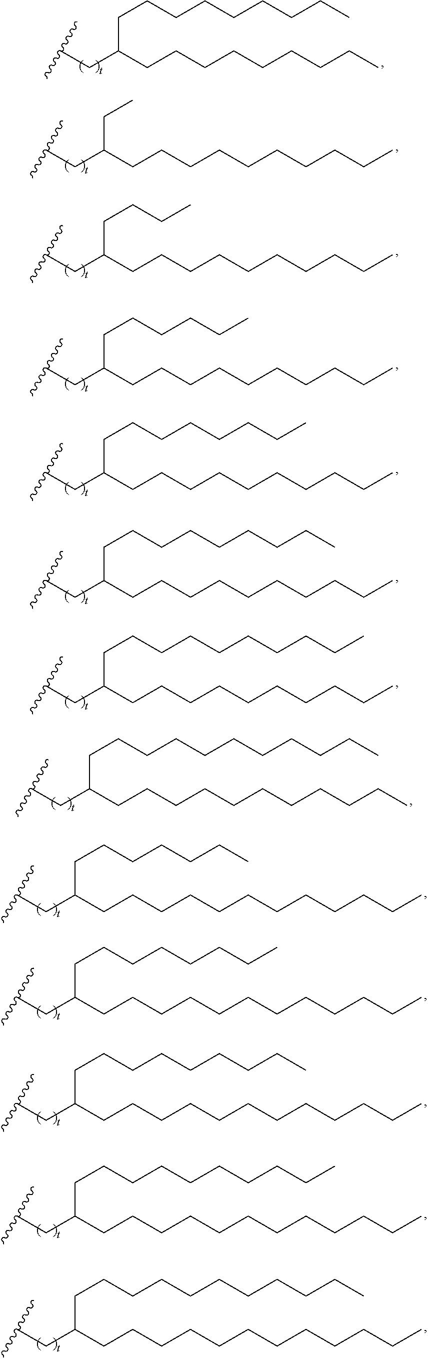

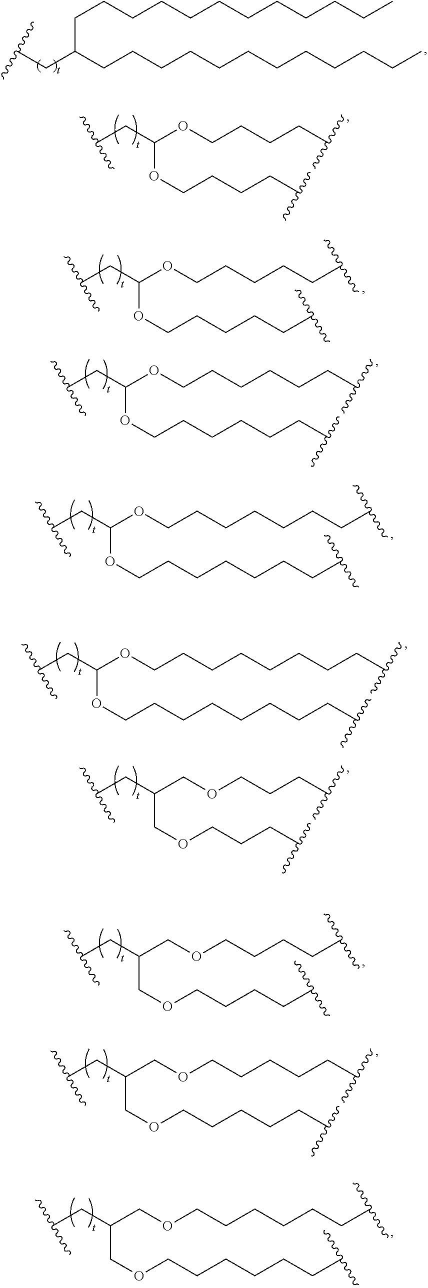

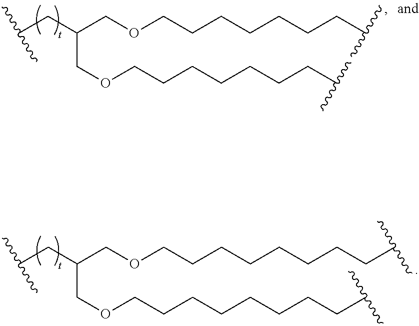

3. The cationic lipid according to claim 1, wherein Rₑ and R_{f} in said 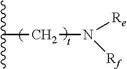
are each independently a C₁₋₁₅ alkyl group, selected from the group consisting of a methyl group, an ethyl group, a propyl group, a butyl group, a pentyl group, a hexyl group, a heptyl group, an octyl group, a nonyl group, and a decyl group; said 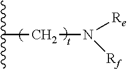
is selected from the following structures:

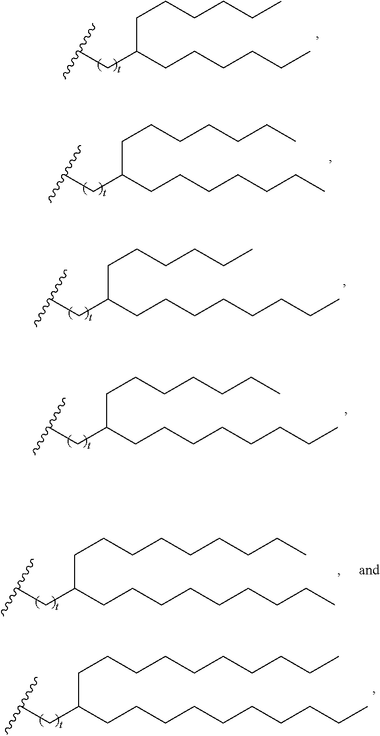

4. The cationic lipid according to claim 1, wherein said B1 and B₂ are selected from the following situations:
Group (1): B₁ and B₂ are each independently a C₁₋₂₀ alkylene group, specifically selected from the group consisting of a methylene group, an ethylene group, a propylene group, a butylene group, a pentylene group, a hexylene group, a heptylene group, an octylene group, a nonylene group, a decylene group, an undecylene group, a dodecylene group, a tridecylene group, a tetradecylene group, a pentadecylene group, a hexadecylene group, a heptadecylene group, an octadecylene group, a nonadecylene group, and an eicosylene group;
Group (2): one of B₁ and B₂ is a linking bond, and the other is a C₁₋₂₀ alkylene group.

5. The cationic lipid according to claim 1, wherein L₁ and L₂ are selected from the following situations:
Group (1): one of L₁ and L₂ is a linking bond, and the other is selected from the group consisting of —OC(═O)—, —C(═O)O—, —OC(═O)O—, —C(═O)—, —O—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—;
Group (2): both L₁ and L₂ are linking bonds;
Group (3): L₁ and L₂ are each independently selected from the group consisting of —OC(═O)—, —C(═O)O—, —OC(═O)O—, —C(═O)—, —O—, —O(CH₂)ₛO—, —S—, —C(═O)S—, —SC(═O)—, —NHC(═O)—, —C(═O)NH—, —NHC(═O)NH—, —OC(═O)NH—, —NHC(═O)O—, —SC(═O)NH—, and —NHC(═O)S—.

6. The cationic lipid according to claim 5, wherein said Li and L₂ are each independently selected from the group consisting of —OC(═O)—, —C(═O)O—, and —OC(═O)O—.

7. The cationic lipid according to claim 1, wherein L₃ is a divalent linking group, selected from the divalent linking group formed by any one, two, or two more divalent linking groups selected from L₄, L₅, and Z; wherein, said L₄ and L₅ are carbon linking groups, which are each independently represented by —(CRₐR_{b})ₜ—(CRₐR_{b})ₒ—(CRₐR_{b})ₚ—, t, o, and p are each independently an integer from 0 to 12, not being 0 simultaneously; Rₐ and R_{b} are, at each occurrence, independently a hydrogen atom or a C₁₋₁₂ alkyl group; said Z is, at each occurrence, independently selected from the group consisting of —C(═O)—, —OC(═O)—, —C(═O)O—, —OC(═O)O—, —O—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—, wherein R_{c} is, at each occurrence, independently a hydrogen atom or a C₁₋₁₂ alkyl group.

8. The cationic lipid according to claim 7, wherein said L₃ is selected from the group consisting of —(CH₂)ₜ—, —(CH₂)ₜZ—, —Z(CH₂)ₜ—, —(CH₂)ₜZ(CH₂)ₜ—, and —Z(CH₂)ₜZ—, wherein t is an integer from 1 to 12; more specifically L₃ is selected from the group consisting of —(CH₂)ₜ—, —(CH₂)ₜO—, —(CH₂)ₜC(═O)—, —(CH₂)ₜC(═O)O—, —(CH₂)ₜOC(═O)—, —(CH₂)ₜC(═O)NH—, —(CH₂)ₜNHC(═O)—, —(CH₂)ₜOC(═O)O—, —(CH₂)ₜNHC(═O)O—, —(CH₂)ₜOC(═O)NH—, —(CH₂)ₜNHC(═O)NH—, —O(CH₂)ₜ—, —C(═O)(CH₂)ₜ—, —C(═O)O(CH₂)ₜ—, —OC(═O)(CH₂)ₜ—, —C(═O)NH(CH₂)ₜ—, —NHC(═O)(CH₂)ₜ—, —OC(═O)O(CH₂)ₜ—, —NHC(═O)O(CH₂)ₜ—, —OC(═O)NH(CH₂)ₜ—, —NHC(═O)NH(CH₂)ₜ—, —(CH₂)ₜO(CH₂)ₜ—, —(CH₂)ₜC(═O)(CH₂)ₜ—, —(CH₂)ₜC(═O)O(CH₂)ₜ—, —(CH₂)ₜOC(═O)(CH₂)ₜ—, —(CH₂)ₜC(═O)NH(CH₂)ₜ—, —(CH₂)ₜNHC(═O)(CH₂)ₜ—, —(CH₂)ₜOC(═O)O(CH₂)ₜ—, —(CH₂)ₜNHC(═O)O(CH₂)ₜ—, —(CH₂)ₜOC(═O)NH(CH₂)ₜ—, —(CH₂)ₜNHC(═O)NH(CH₂)ₜ—, —O(CH₂)ₜO—, —C(═O)(CH₂)ₜC(═O)—, —C(═O)O(CH₂)ₜC(═O)O—, —OC(═O)(CH₂)ₜOC(═O)—, —C(═O)O(CH₂)ₜOC(═O)—, —OC(═O)(CH₂)ₜC(═O)O—, —OC(═O)O(CH₂)ₜOC(═O)O—, —C(═O)NH(CH₂)ₜC(═O)NH—, —NHC(═O)(CH₂)ₜNHC(═O)—, —NHC(═O)(CH₂)ₜC(═O)NH—, —C(═O)NH(CH₂)ₜNHC(═O)—, —NHC(═O)O(CH₂)ₜNHC(═O)O—, —OC(═O)NH(CH₂)ₜOC(═O)NH—, —NHC(═O)O(CH₂)ₜOC(═O)NH—, —OC(═O)NH(CH₂)ₜNHC(═O)O—, —NHC(═O)NH(CH₂)ₜNHC(═O)NH—, —C(═O)(CH₂)ₜO—, —C(═O)(CH₂)ₜC(═O)O—, —C(═O)(CH₂)ₜOC(═O)—, —C(═O)(CH₂)ₜOC(═O)O—, —C(═O)(CH₂)ₜNHC(═O)O—, —C(═O)(CH₂)ₜOC(═O)NH—, and —C(═O)(CH₂)ₜNHC(═O)NH—.

9. The cationic lipid according to claim 1, wherein said R₃ is a hydrogen atom, —R_{d}, —OR_{d}, —C(═O)R_{d}—, —C(═O)OR_{d}, —OC(═O)R_{d}, —OC(═O)OR_{d}, or 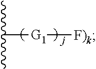
specifically it is selected from the group consisting of H, —(CH₂)ₜOH, —(CH₂)ₜSH, —OCH₃, —OCH₂CH₃, —(CH₂)ₜNH₂, —(CH₂)ₜC(═O)OH, —C(═O)(CH₂)ₜC(═O)OH, —C(═O)CH₃, —(CH₂)N₃, —C(═O)CH₂CH₃, —C(═O)OCH₃, —OC(═O)OCH₃, —C(═O)OCH₂CH₃, —OC(═O)OCH₂CH₃, —(CH₂)ₜN(CH₃)₂, —(CH₂)ₜN(CH₂CH₃)₂, —(CH₂)ₜCHO, 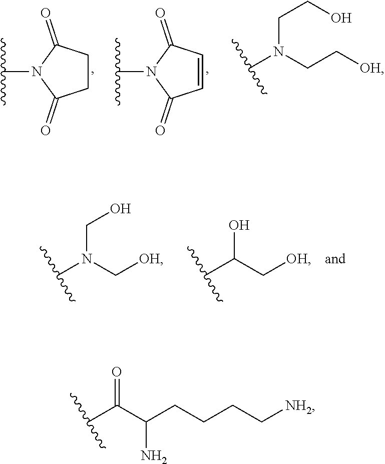
wherein, t is an integer from 1 to 12.

10. The cationic lipid according to claim 5, wherein the structure of said cationic lipid is selected from the group consisting of the following structural formulas: 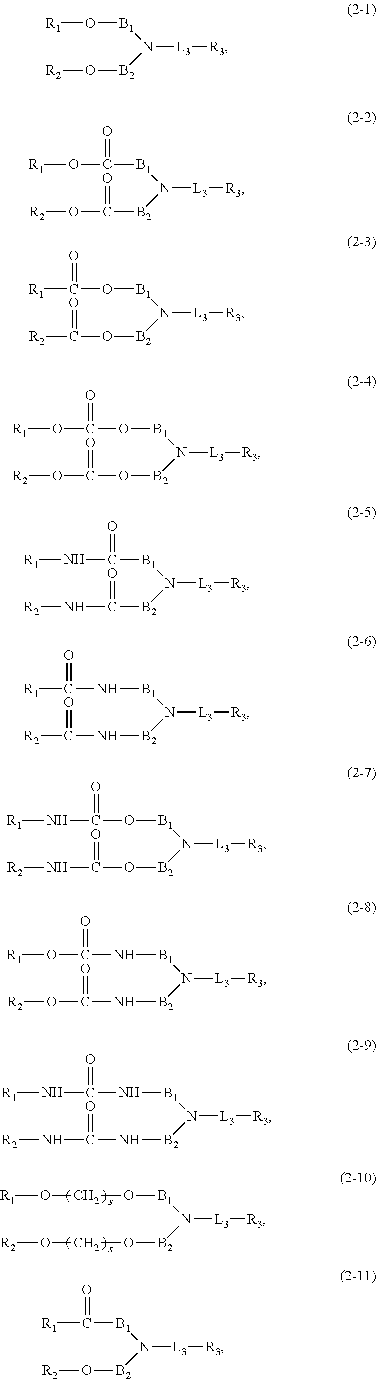 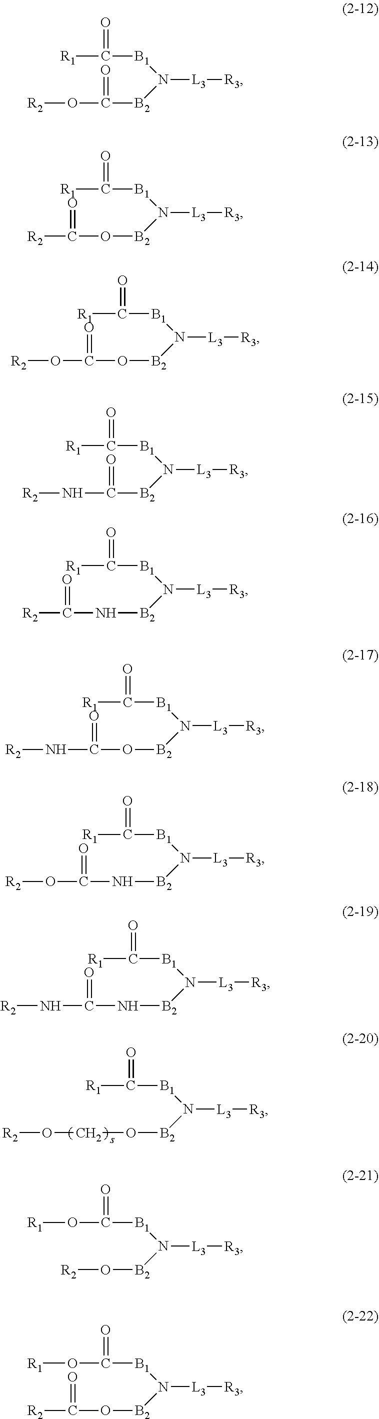 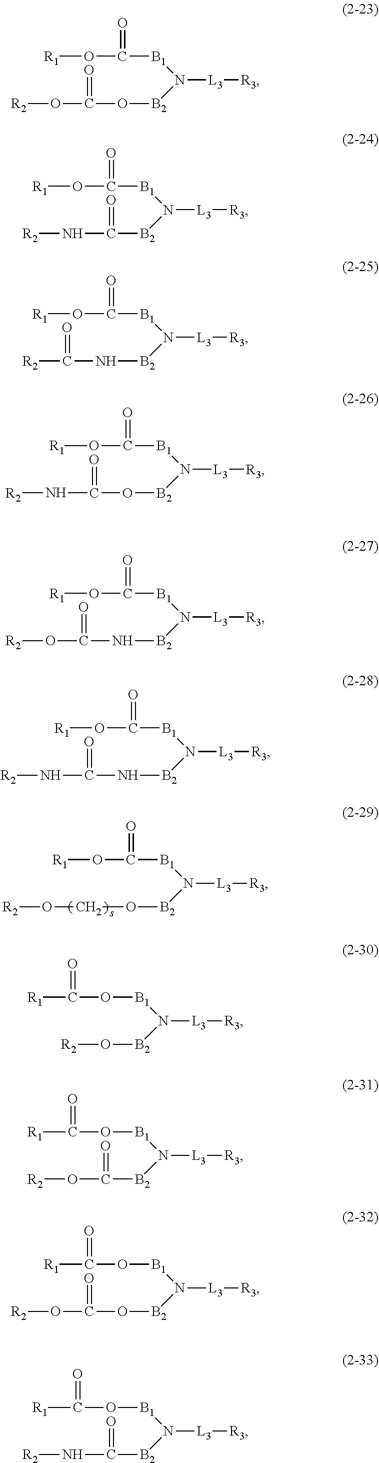 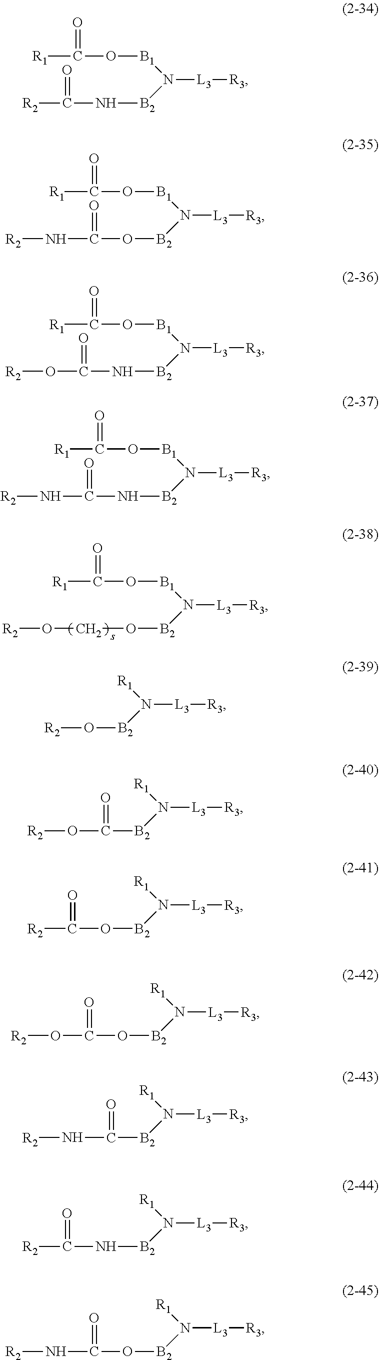 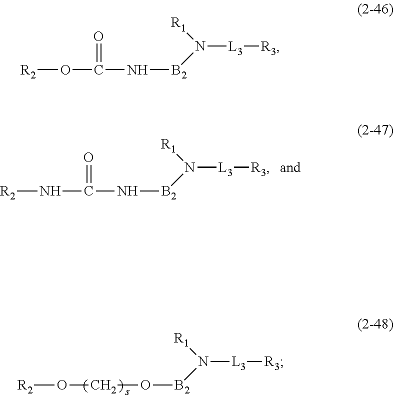
wherein, in the formula (2-39) to formula (2-48), R₁ is, at each occurrence, independently a C₁₋₃₀ aliphatic hydrocarbon group or a residue of C₁₋₃₀ aliphatic hydrocarbon derivative, and R₂ is, at each occurrence, independently represented by

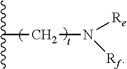

11. The cationic lipid according to claim 1, wherein the cationic lipid is selected from the group consisting of the following structures:

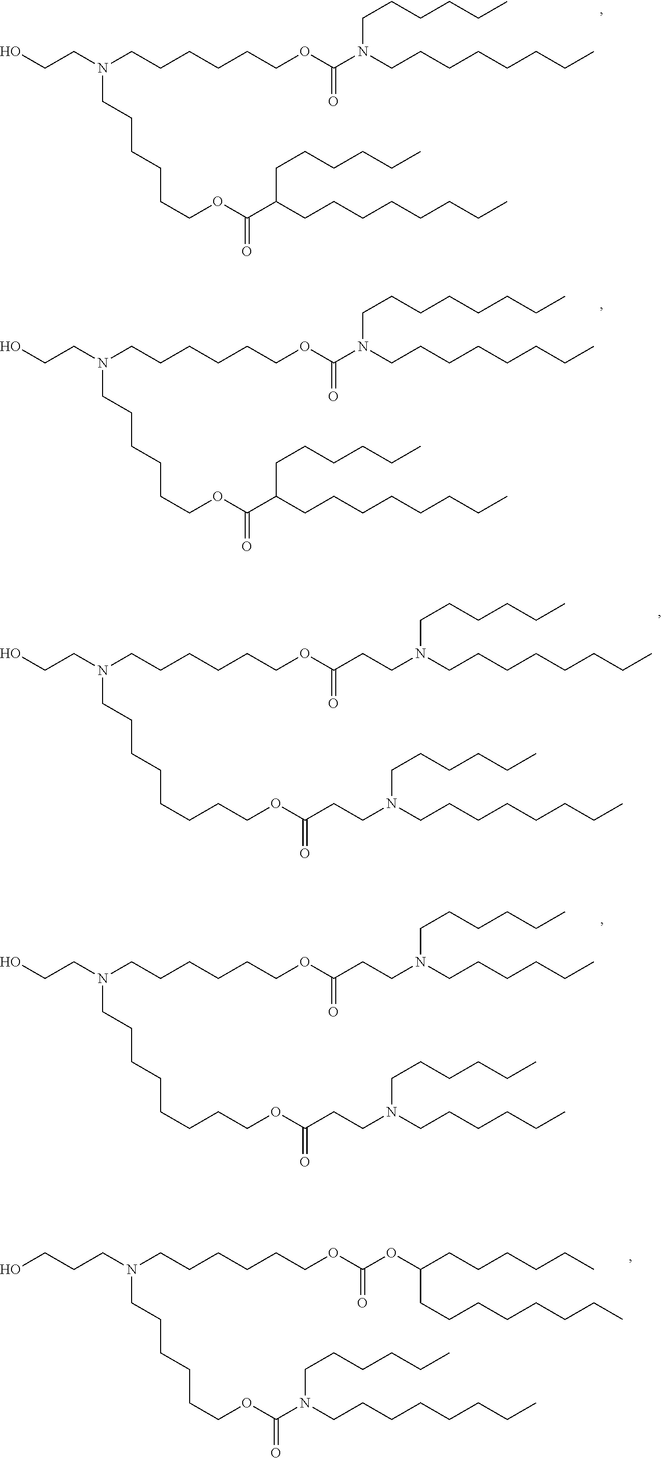 [SMILES: CCCCCCCCC(CCCCCC)C(=O)OCCCCCCN(CCO)CCCCCCOC(=O)N(CCCCCC)CCCCCCCC ; CCCCCCCCC(CCCCCC)C(=O)OCCCCCCN(CCO)CCCCCCOC(=O)N(CCCCCCCC)CCCCCCCC ; CCCCCCCCN(CCCCCC)CCC(=O)OCCCCCCCCN(CCO)CCCCCCOC(=O)CCN(CCCCCC)CCCCCCCC ; CCCCCCN(CCCCCC)CCC(=O)OCCCCCCCCN(CCO)CCCCCCOC(=O)CCN(CCCCCC)CCCCCC ; CCCCCCCCC(CCCCCC)OC(=O)OCCCCCCN(CCCO)CCCCCCOC(=O)N(CCCCCC)CCCCCCCC]

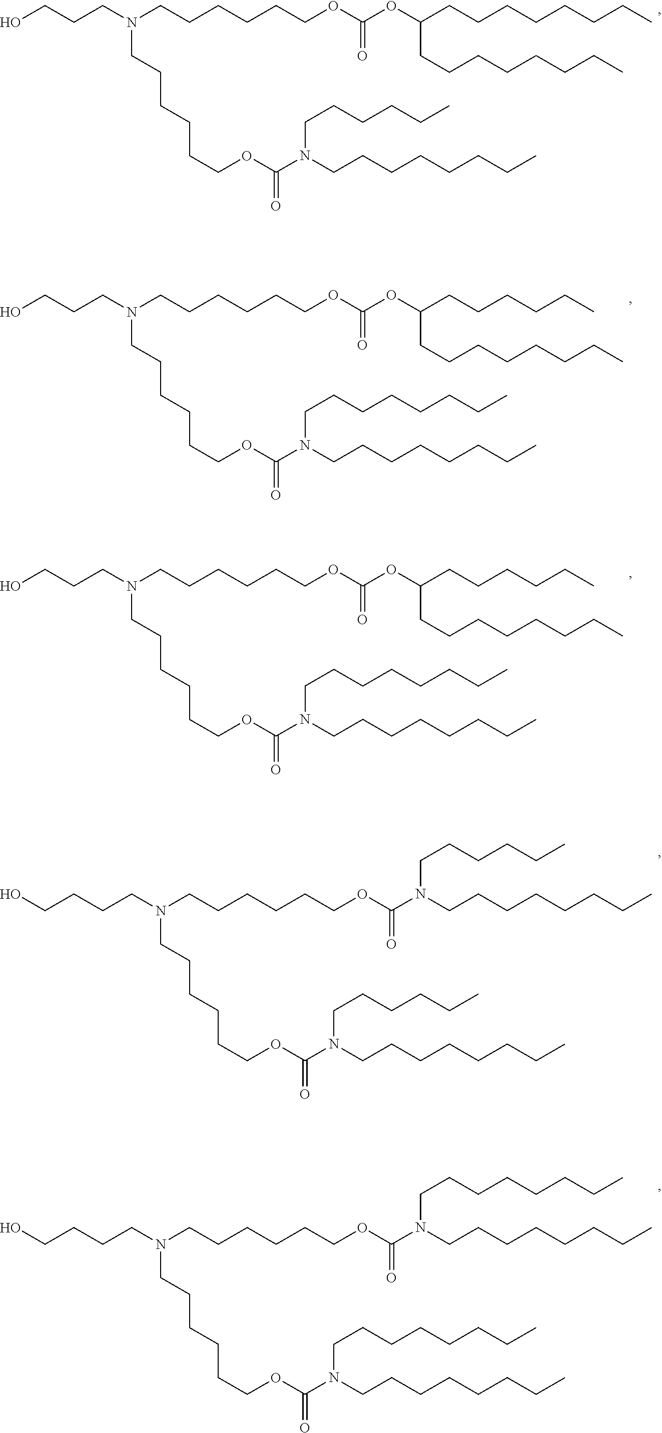 [SMILES: CCCCCCCCC(CCCCCCCC)OC(=O)OCCCCCCN(CCCO)CCCCCCOC(=O)N(CCCCCC)CCCCCCCC ; CCCCCCCCC(CCCCCC)OC(=O)OCCCCCCN(CCCO)CCCCCCOC(=O)N(CCCCCCCC)CCCCCCCC ; CCCCCCCCC(CCCCCC)OC(=O)OCCCCCCN(CCCO)CCCCCCOC(=O)N(CCCCCCCC)CCCCCCCC ; CCCCCCCCN(CCCCCC)C(=O)OCCCCCCN(CCCCO)CCCCCCOC(=O)N(CCCCCC)CCCCCCCC ; CCCCCCCCN(CCCCCCCC)C(=O)OCCCCCCN(CCCCO)CCCCCCOC(=O)N(CCCCCCCC)CCCCCCCC]

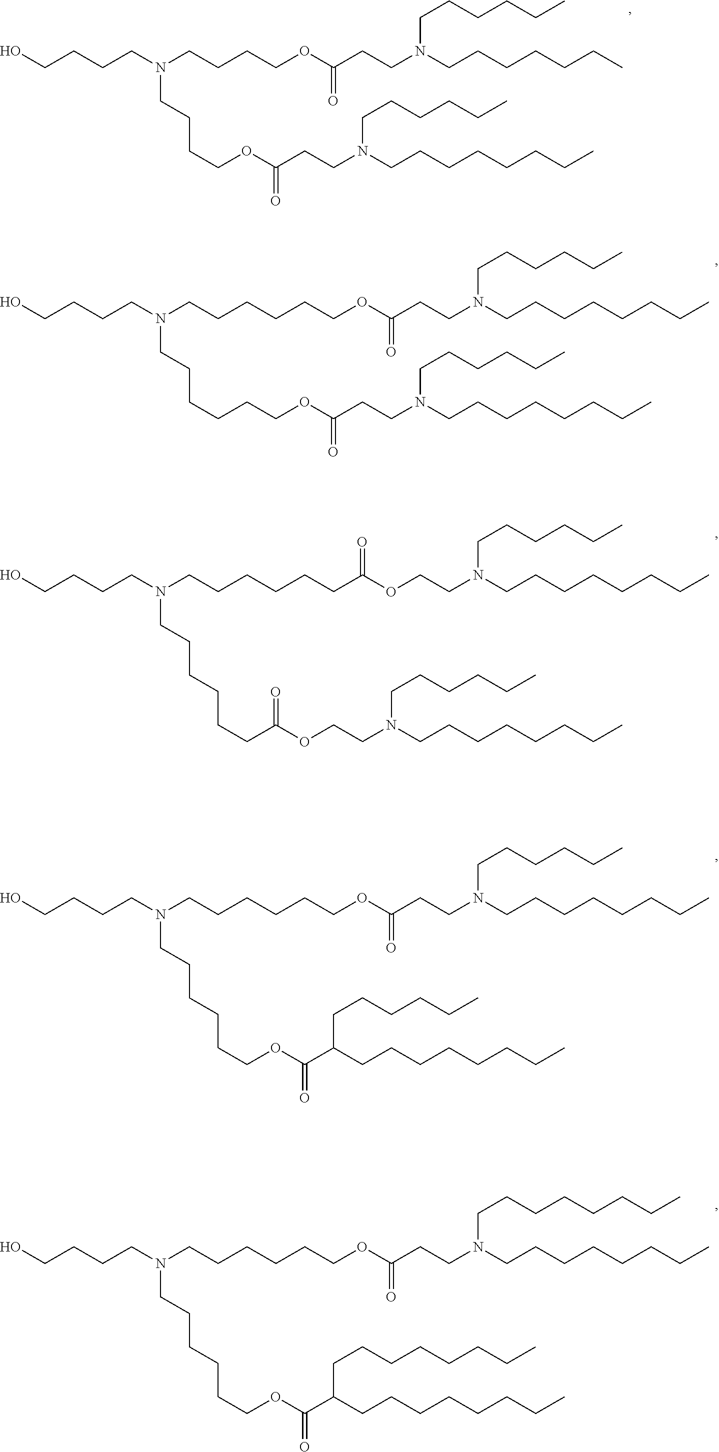 [SMILES: CCCCCCCCN(CCCCCC)CCC(=O)OCCCCN(CCCCO)CCCCOC(=O)CCN(CCCCCC)CCCCCCC ; CCCCCCCCN(CCCCCC)CCC(=O)OCCCCCCN(CCCCO)CCCCCCOC(=O)CCN(CCCCCC)CCCCCCCC ; CCCCCCCCN(CCCCCC)CCOC(=O)CCCCCCN(CCCCO)CCCCCCC(=O)OCCN(CCCCCC)CCCCCCCC ; CCCCCCCCC(CCCCCC)C(=O)OCCCCCCN(CCCCO)CCCCCCOC(=O)CCN(CCCCCC)CCCCCCCC ; CCCCCCCCC(CCCCCCCC)C(=O)OCCCCCCN(CCCCO)CCCCCCOC(=O)CCN(CCCCCCCC)CCCCCCCC]

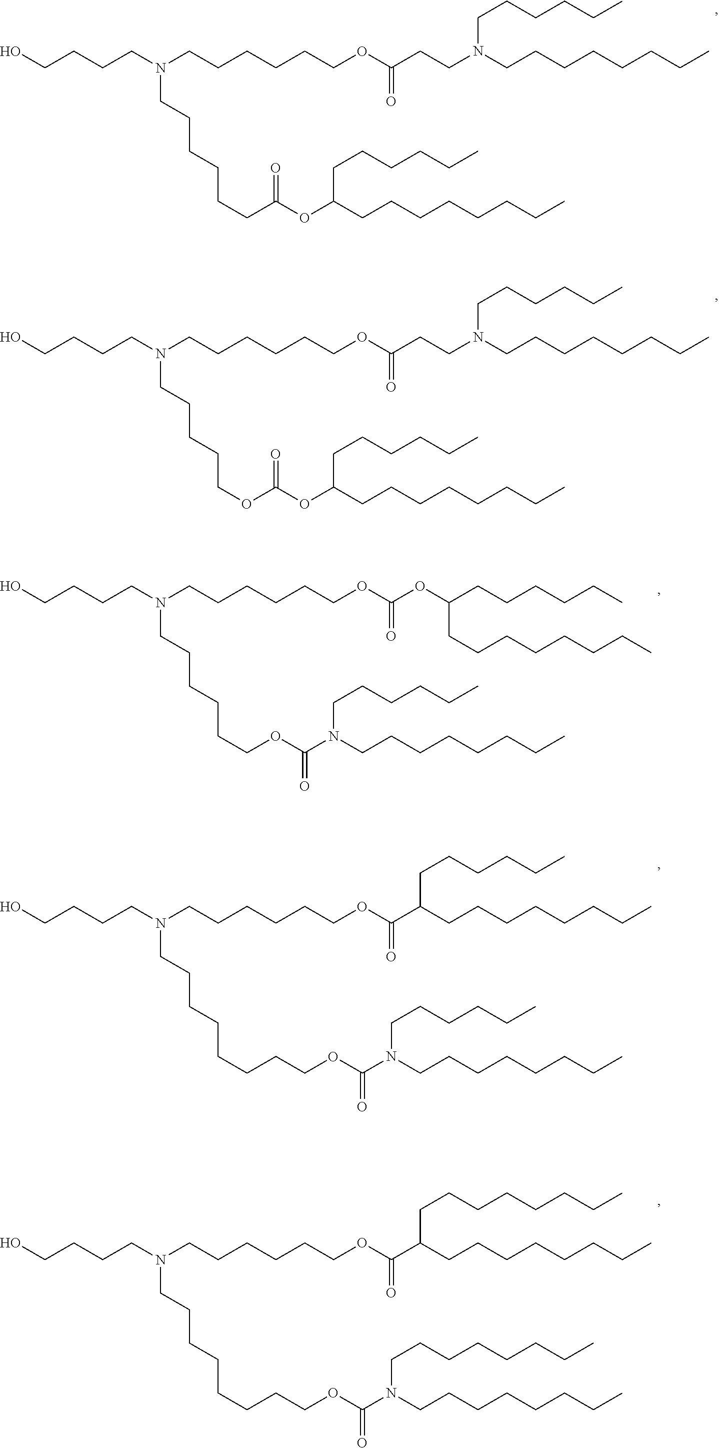 [SMILES: CCCCCCCCC(CCCCCC)OC(=O)CCCCCCN(CCCCO)CCCCCCOC(=O)CCN(CCCCCC)CCCCCCCC ; CCCCCCCCC(CCCCCC)OC(=O)OCCCCCN(CCCCO)CCCCCCOC(=O)CCN(CCCCCC)CCCCCCCC ; CCCCCCCCC(CCCCCC)OC(=O)OCCCCCCN(CCCCO)CCCCCCOC(=O)N(CCCCCC)CCCCCCCC ; CCCCCCCCC(CCCCCC)C(=O)OCCCCCCN(CCCCO)CCCCCCCCOC(=O)N(CCCCCC)CCCCCCCC ; CCCCCCCCC(CCCCCCCC)C(=O)OCCCCCCN(CCCCO)CCCCCCCCOC(=O)N(CCCCCCCC)CCCCCCCC]

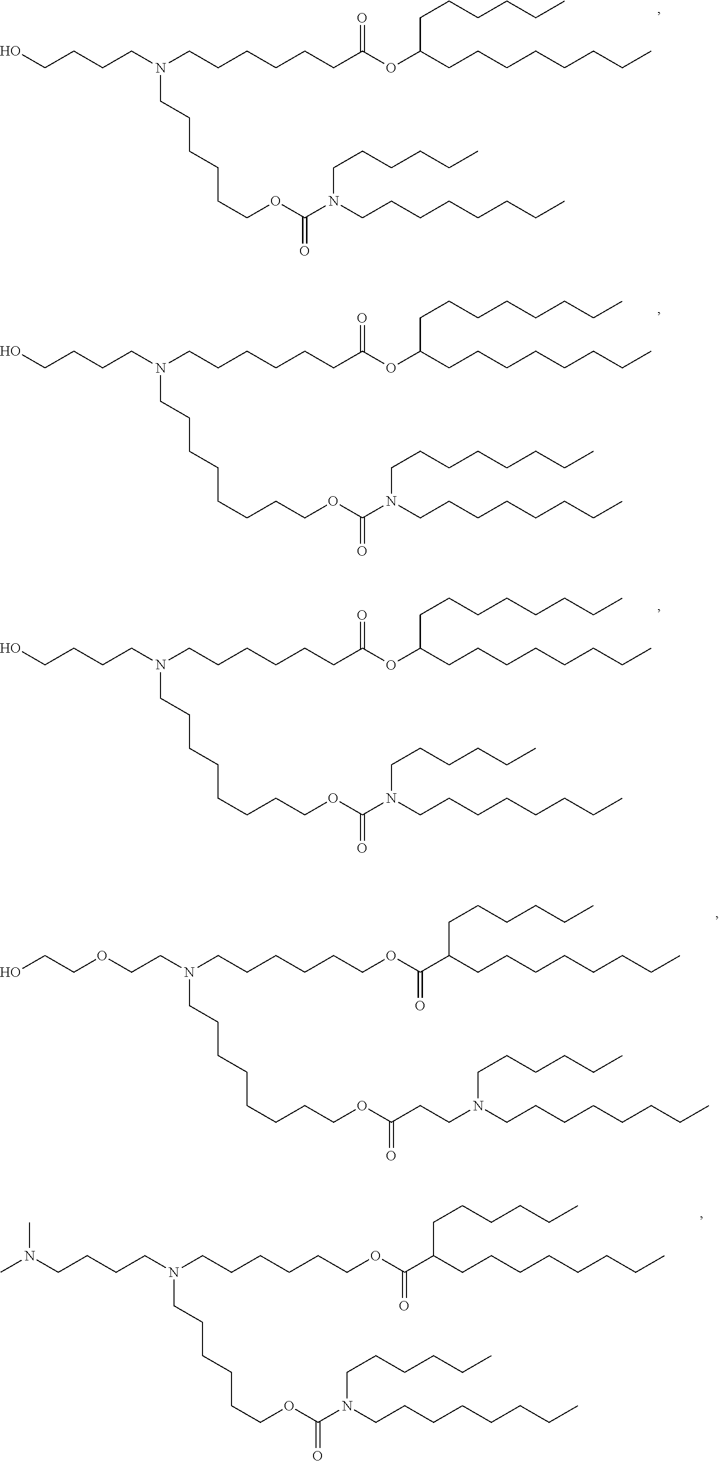

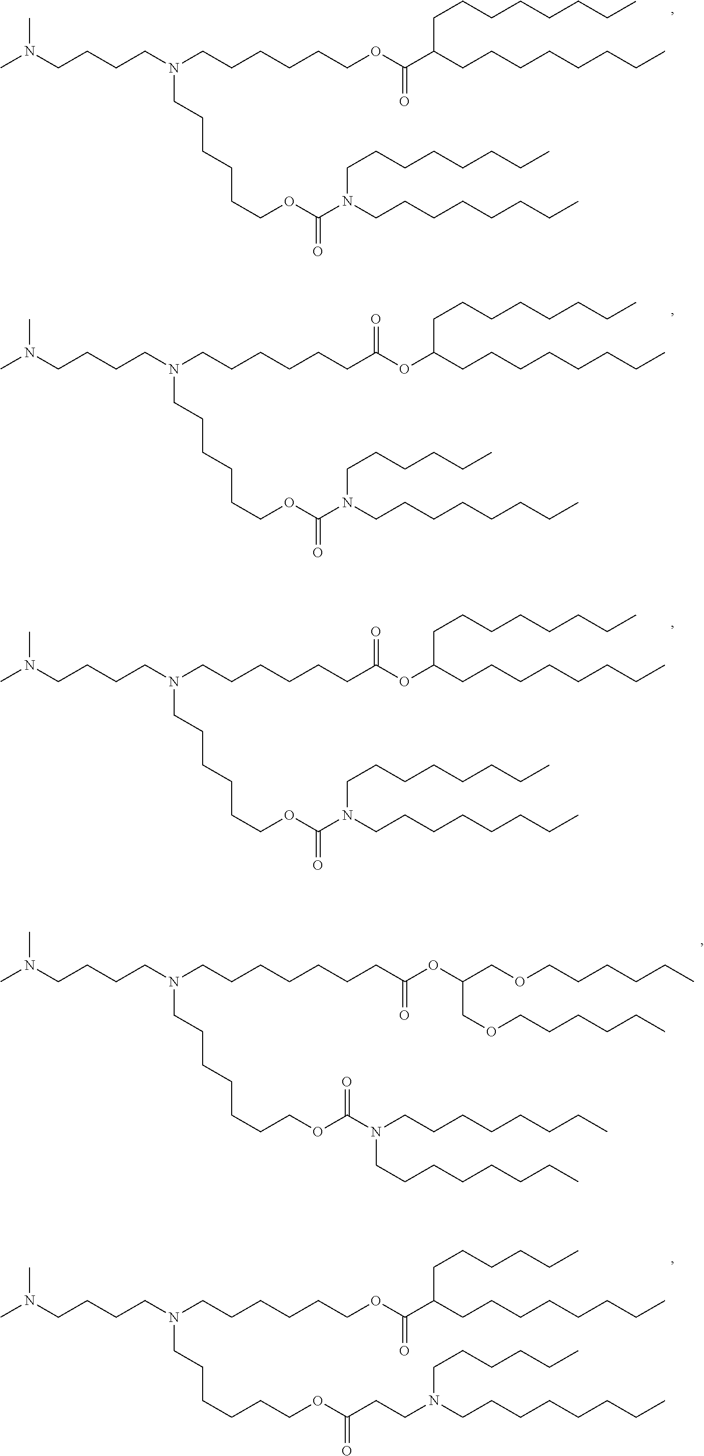

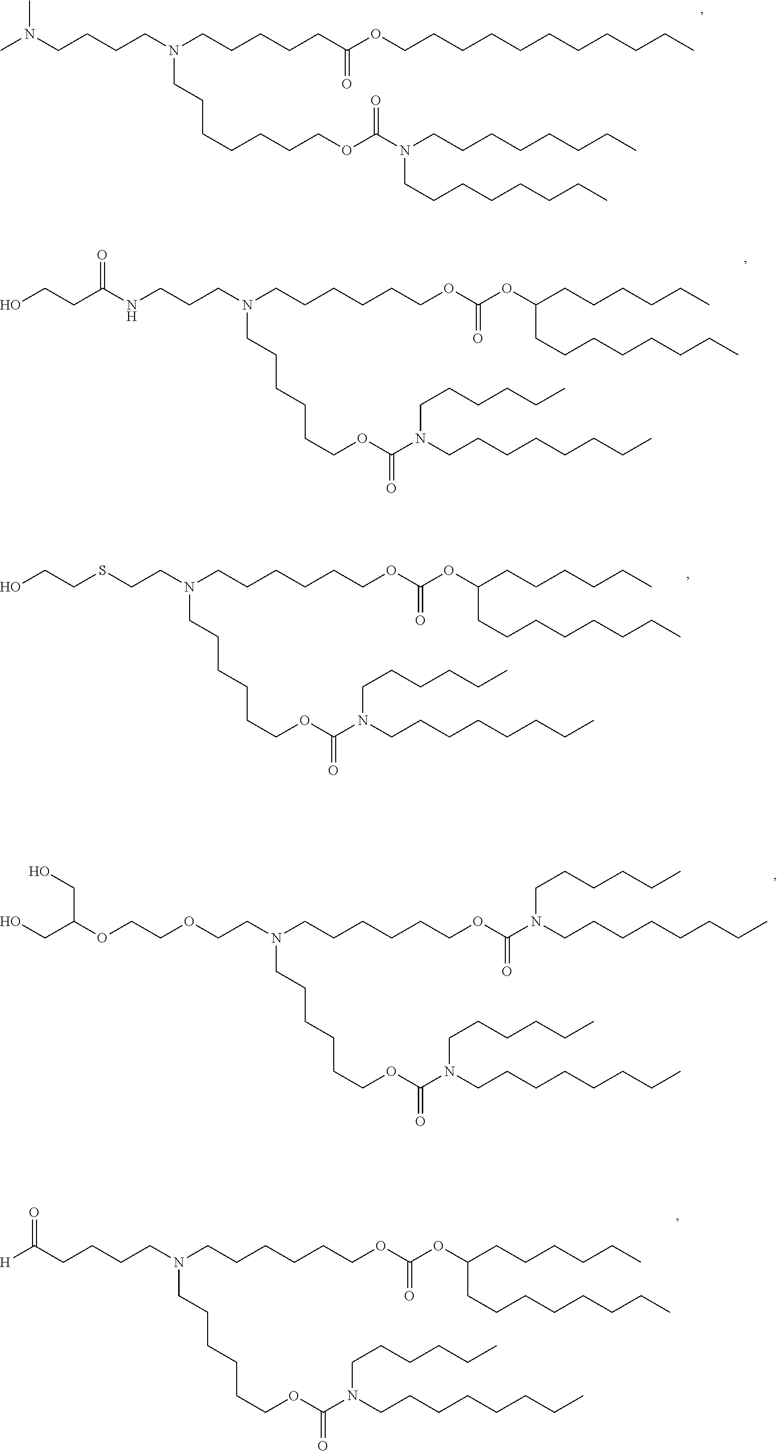 [SMILES: CCCCCCCCCCCOC(=O)CCCCCN(CCCCCCCOC(=O)N(CCCCCCCC)CCCCCCCC)CCCCN(C)C ; CCCCCCCCC(CCCCCC)OC(=O)OCCCCCCN(CCCCCCOC(=O)N(CCCCCC)CCCCCCCC)CCCNC(=O)CCO ; CCCCCCCCC(CCCCCC)OC(=O)OCCCCCCN(CCCCCCOC(=O)N(CCCCCC)CCCCCCCC)CCSCCO ; CCCCCCCCN(CCCCCC)C(=O)OCCCCCCN(CCCCCCOC(=O)N(CCCCCC)CCCCCCCC)CCOCCOC(CO)CO ; CCCCCCCCC(CCCCCC)OC(=O)OCCCCCCN(CCCCC=O)CCCCCCOC(=O)N(CCCCCC)CCCCCCCC]

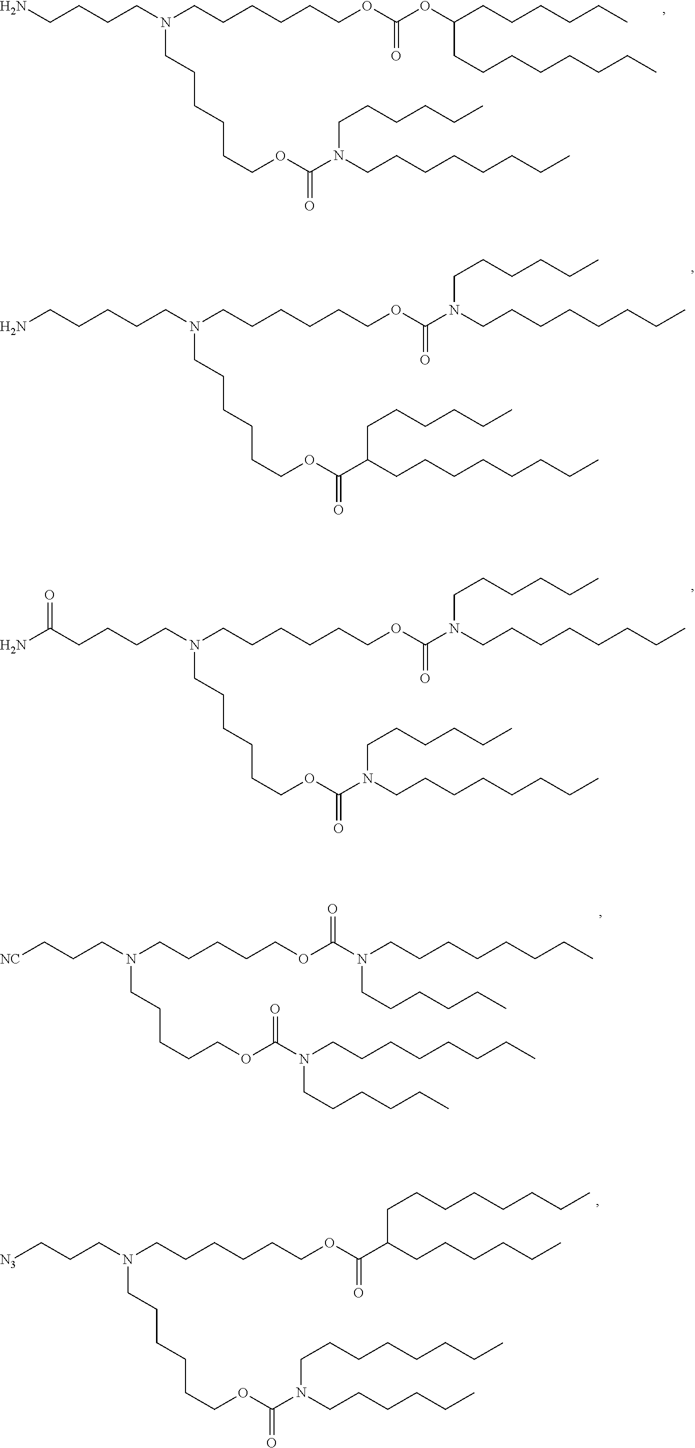 [SMILES: CCCCCCCCC(CCCCCC)OC(=O)OCCCCCCN(CCCCN)CCCCCCOC(=O)N(CCCCCC)CCCCCCCC ; CCCCCCCCC(CCCCCC)C(=O)OCCCCCCN(CCCCCN)CCCCCCOC(=O)N(CCCCCC)CCCCCCCC ; CCCCCCCCN(CCCCCC)C(=O)OCCCCCCN(CCCCCCOC(=O)N(CCCCCC)CCCCCCCC)CCCCC(N)=O ; CCCCCCCCN(CCCCCC)C(=O)OCCCCCN(CCCC#N)CCCCCOC(=O)N(CCCCCC)CCCCCCCC ; CCCCCCCCC(CCCCCC)C(=O)OCCCCCCN(CCCCCCOC(=O)N(CCCCCC)CCCCCCCC)CCCN=[N+]=[N-]]

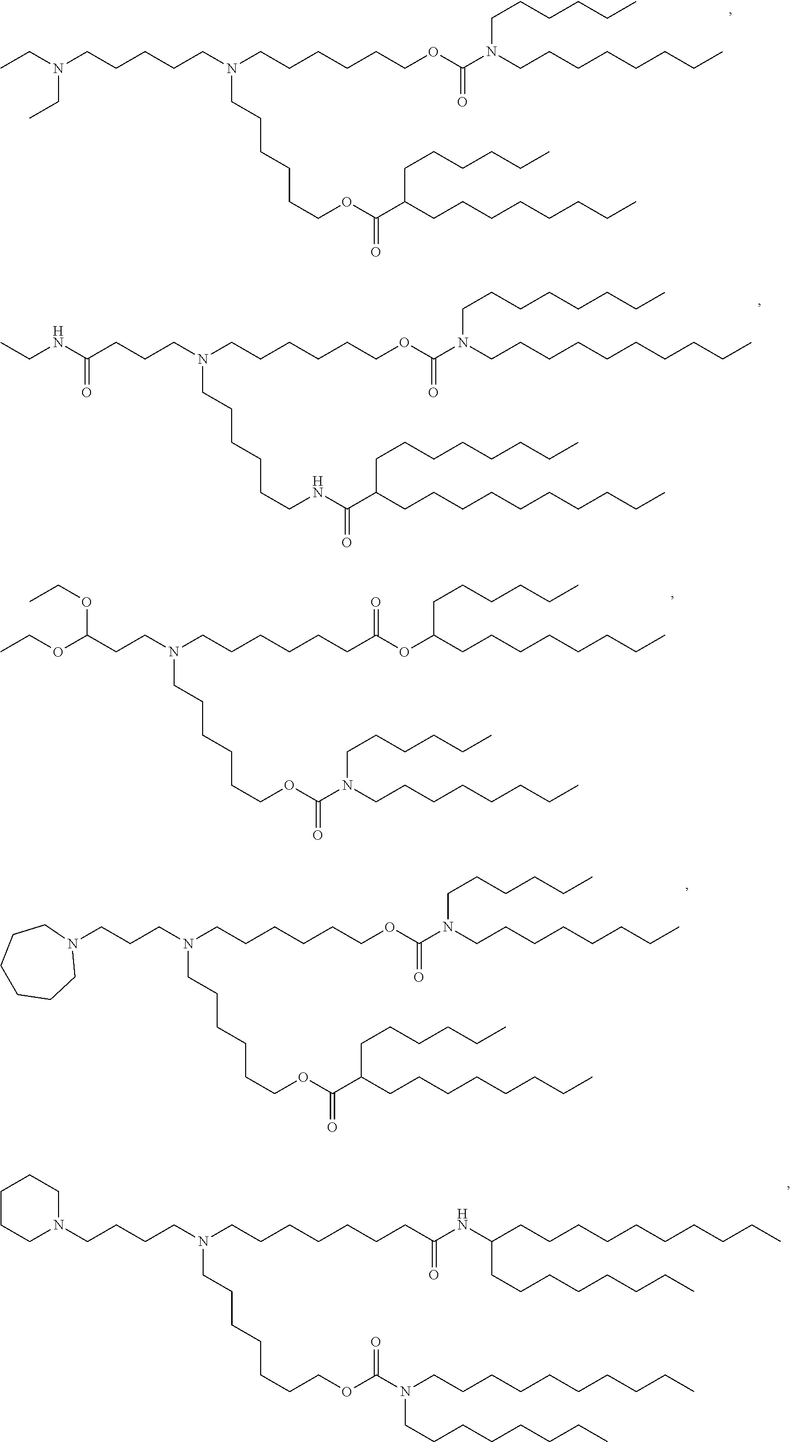

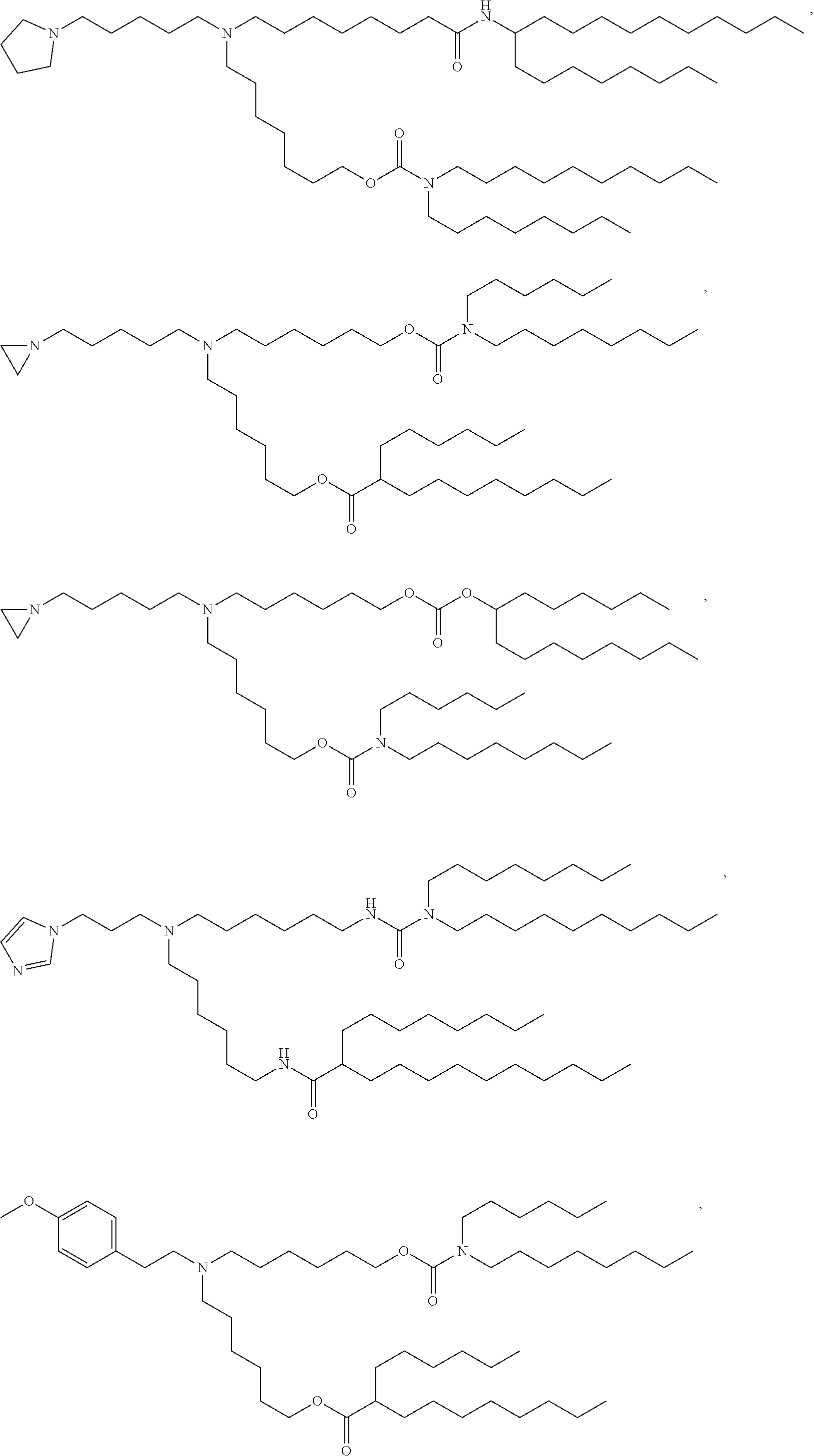

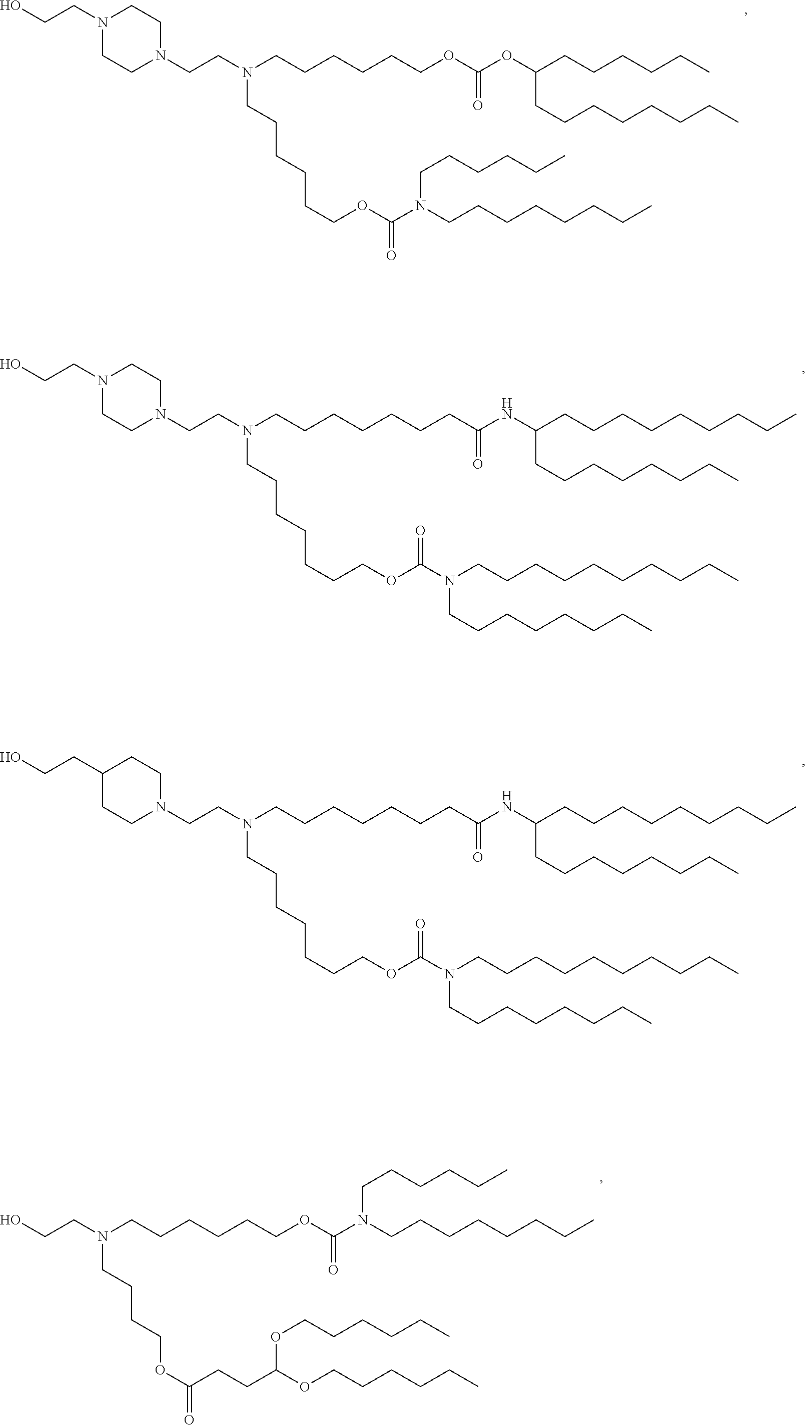 [SMILES: CCCCCCCCC(CCCCCC)OC(=O)OCCCCCCN(CCCCCCOC(=O)N(CCCCCC)CCCCCCCC)CCN1CCN(CCO)CC1 ; CCCCCCCCCCN(CCCCCCCC)C(=O)OCCCCCCCN(CCCCCCCC(=O)NC(CCCCCCCC)CCCCCCCCC)CCN1CCN(CCO)CC1 ; CCCCCCCCCCN(CCCCCCCC)C(=O)OCCCCCCCN(CCCCCCCC(=O)NC(CCCCCCCC)CCCCCCCCC)CCN1CCC(CCO)CC1 ; CCCCCCCCN(CCCCCC)C(=O)OCCCCCCN(CCO)CCCCOC(=O)CCC(OCCCCCC)OCCCCCC]

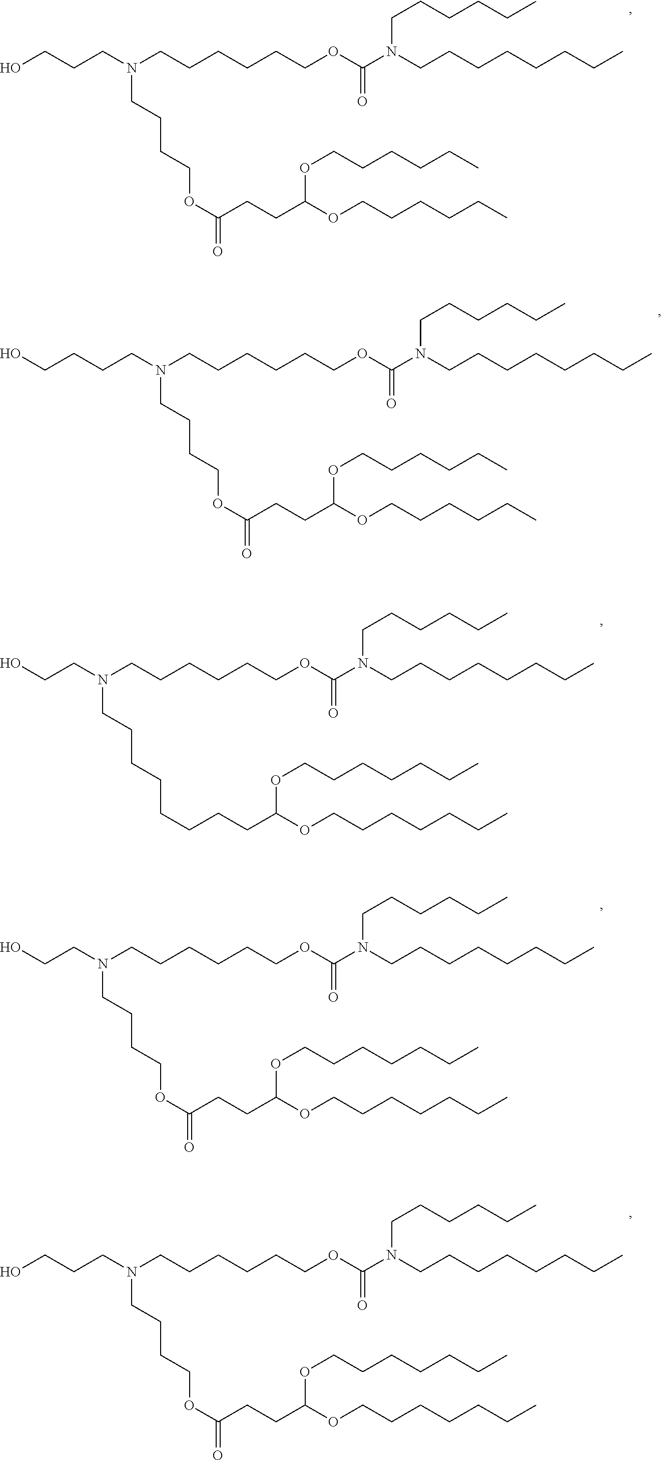 [SMILES: CCCCCCCCN(CCCCCC)C(=O)OCCCCCCN(CCCO)CCCCOC(=O)CCC(OCCCCCC)OCCCCCC ; CCCCCCCCN(CCCCCC)C(=O)OCCCCCCN(CCCCO)CCCCOC(=O)CCC(OCCCCCC)OCCCCCC ; CCCCCCCCN(CCCCCC)C(=O)OCCCCCCN(CCO)CCCCCCCCC(OCCCCCCC)OCCCCCCC ; CCCCCCCCN(CCCCCC)C(=O)OCCCCCCN(CCO)CCCCOC(=O)CCC(OCCCCCCC)OCCCCCCC ; CCCCCCCCN(CCCCCC)C(=O)OCCCCCCN(CCCO)CCCCOC(=O)CCC(OCCCCCCC)OCCCCCCC]

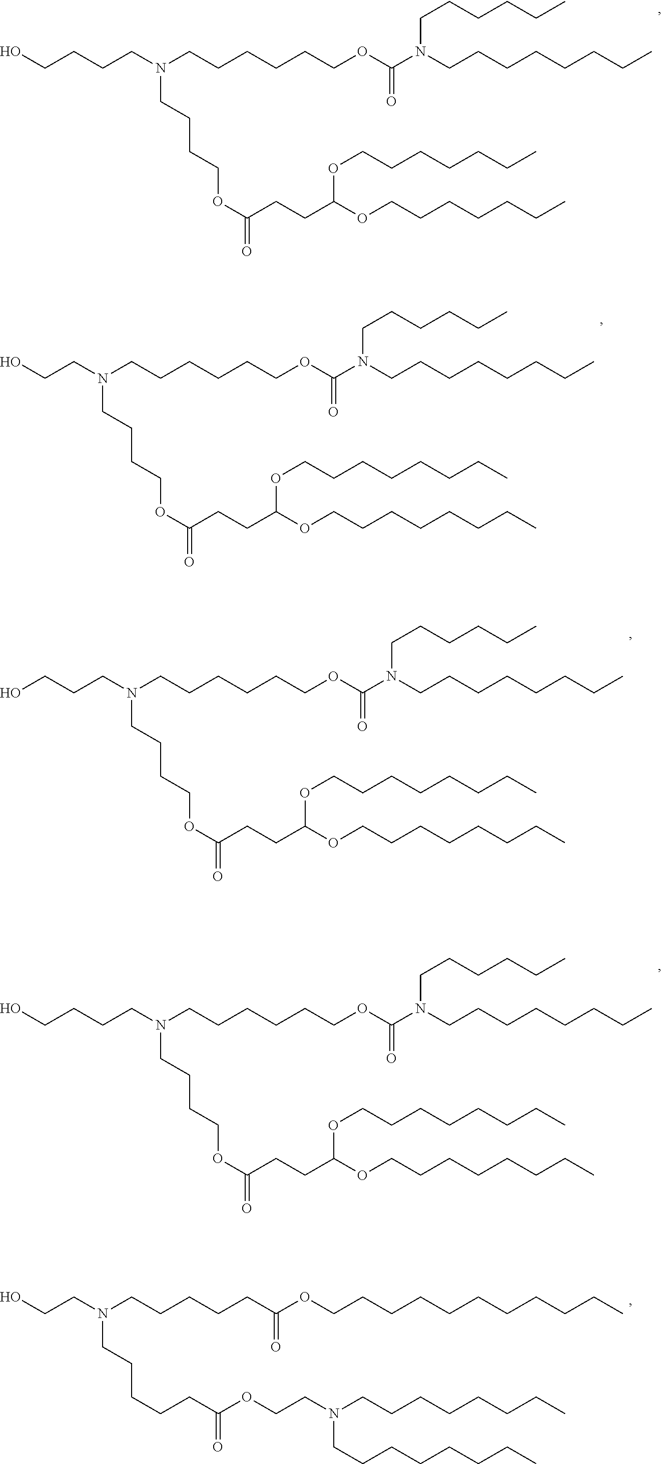 [SMILES: CCCCCCCCN(CCCCCC)C(=O)OCCCCCCN(CCCCO)CCCCOC(=O)CCC(OCCCCCCC)OCCCCCCC ; CCCCCCCCOC(CCC(=O)OCCCCN(CCO)CCCCCCOC(=O)N(CCCCCC)CCCCCCCC)OCCCCCCCC ; CCCCCCCCOC(CCC(=O)OCCCCN(CCCO)CCCCCCOC(=O)N(CCCCCC)CCCCCCCC)OCCCCCCCC ; CCCCCCCCOC(CCC(=O)OCCCCN(CCCCO)CCCCCCOC(=O)N(CCCCCC)CCCCCCCC)OCCCCCCCC ; CCCCCCCCCCCOC(=O)CCCCCN(CCO)CCCCCC(=O)OCCN(CCCCCCCC)CCCCCCCC]

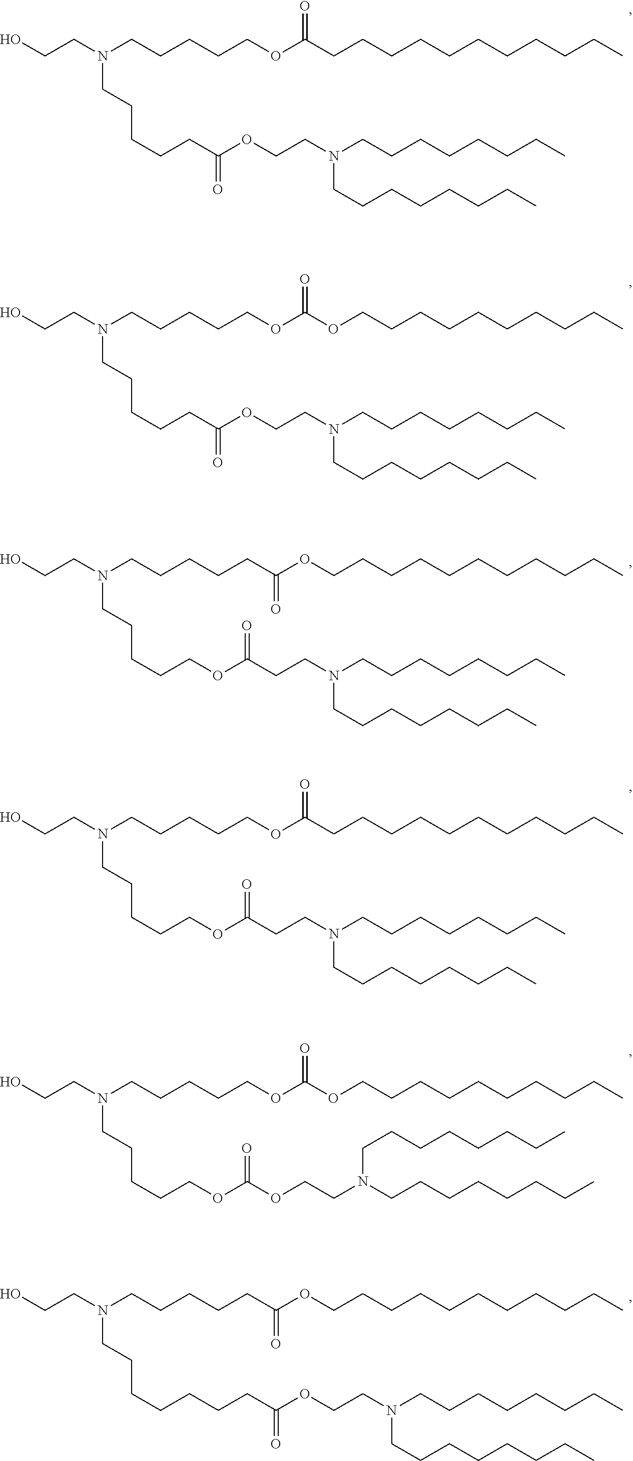 [SMILES: CCCCCCCCCCCC(=O)OCCCCCN(CCO)CCCCCC(=O)OCCN(CCCCCCCC)CCCCCCCC ; CCCCCCCCCCOC(=O)OCCCCCN(CCO)CCCCCC(=O)OCCN(CCCCCCCC)CCCCCCCC ; CCCCCCCCCCCOC(=O)CCCCCN(CCO)CCCCCOC(=O)CCN(CCCCCCCC)CCCCCCCC ; CCCCCCCCCCCC(=O)OCCCCCN(CCO)CCCCCOC(=O)CCN(CCCCCCCC)CCCCCCCC ; CCCCCCCCCCOC(=O)OCCCCCN(CCO)CCCCCOC(=O)OCCN(CCCCCCCC)CCCCCCCC ; CCCCCCCCCCCOC(=O)CCCCCN(CCO)CCCCCCCC(=O)OCCN(CCCCCCCC)CCCCCCCC]

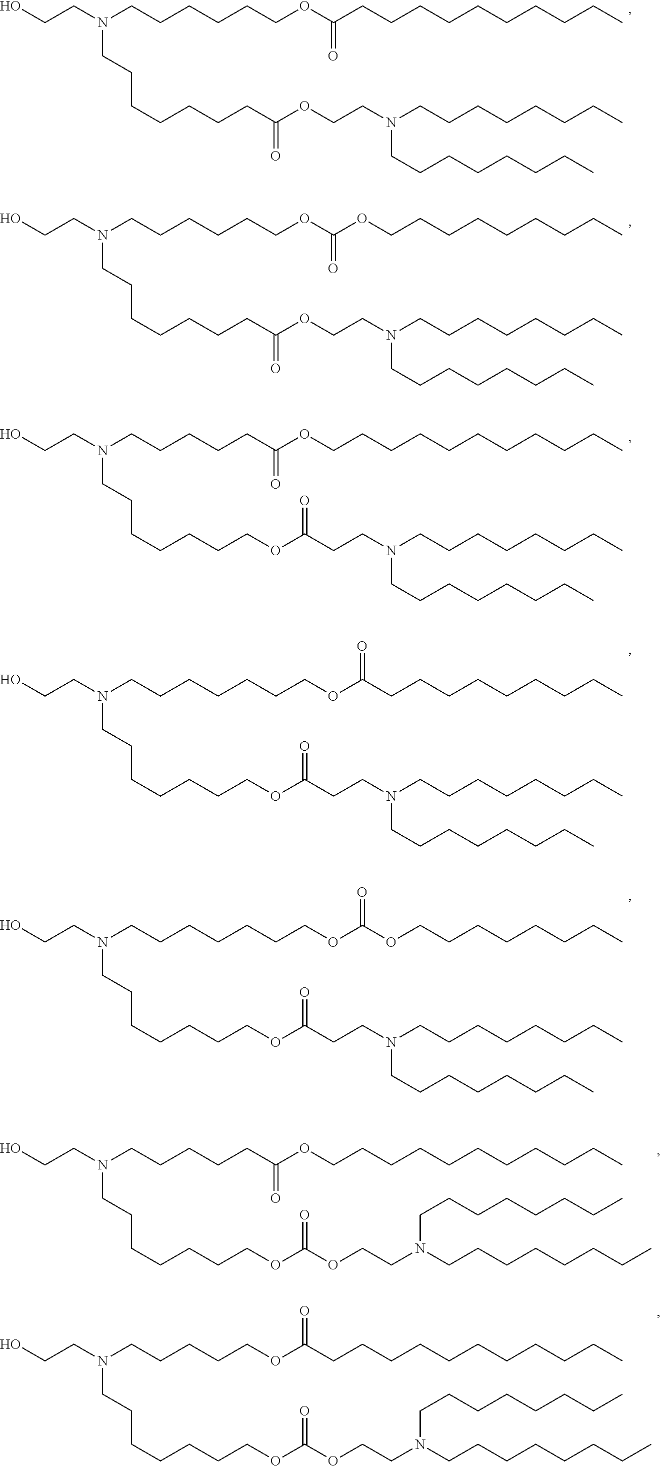

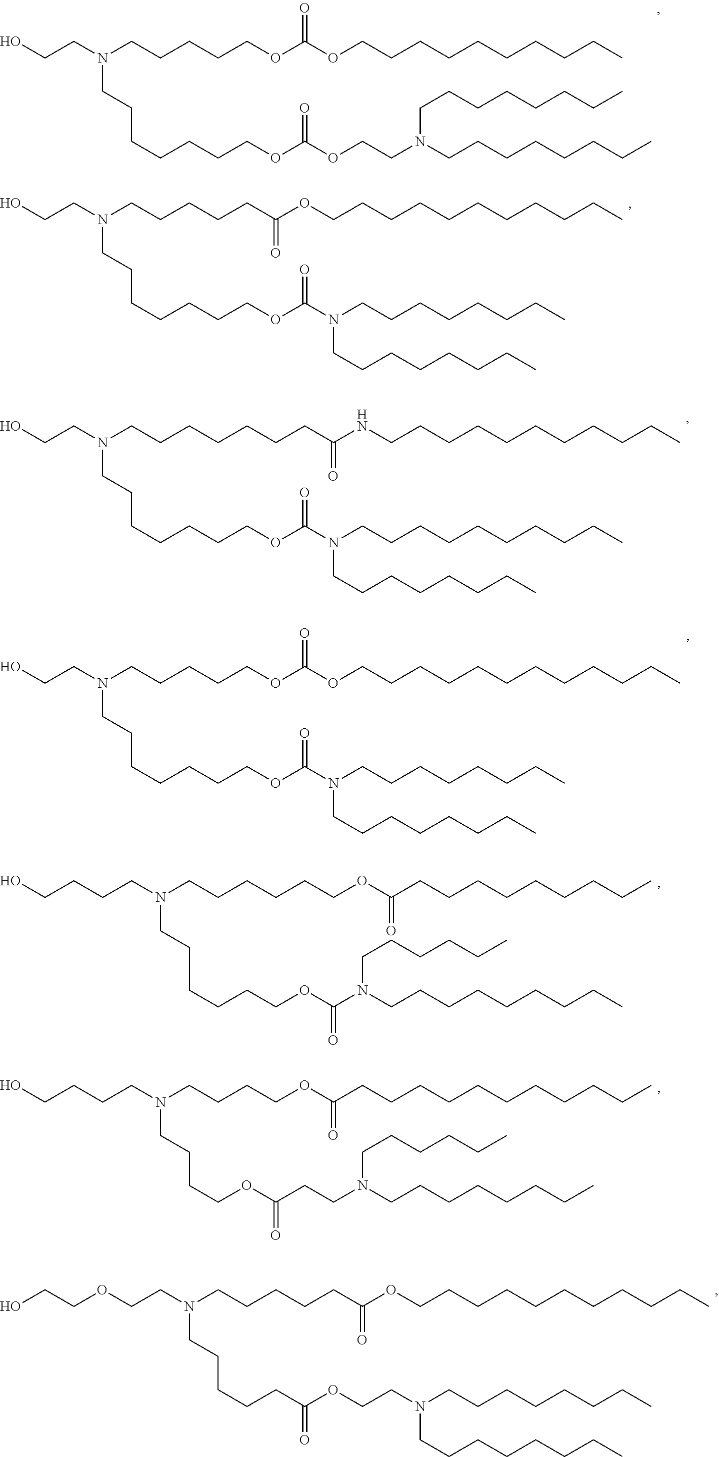

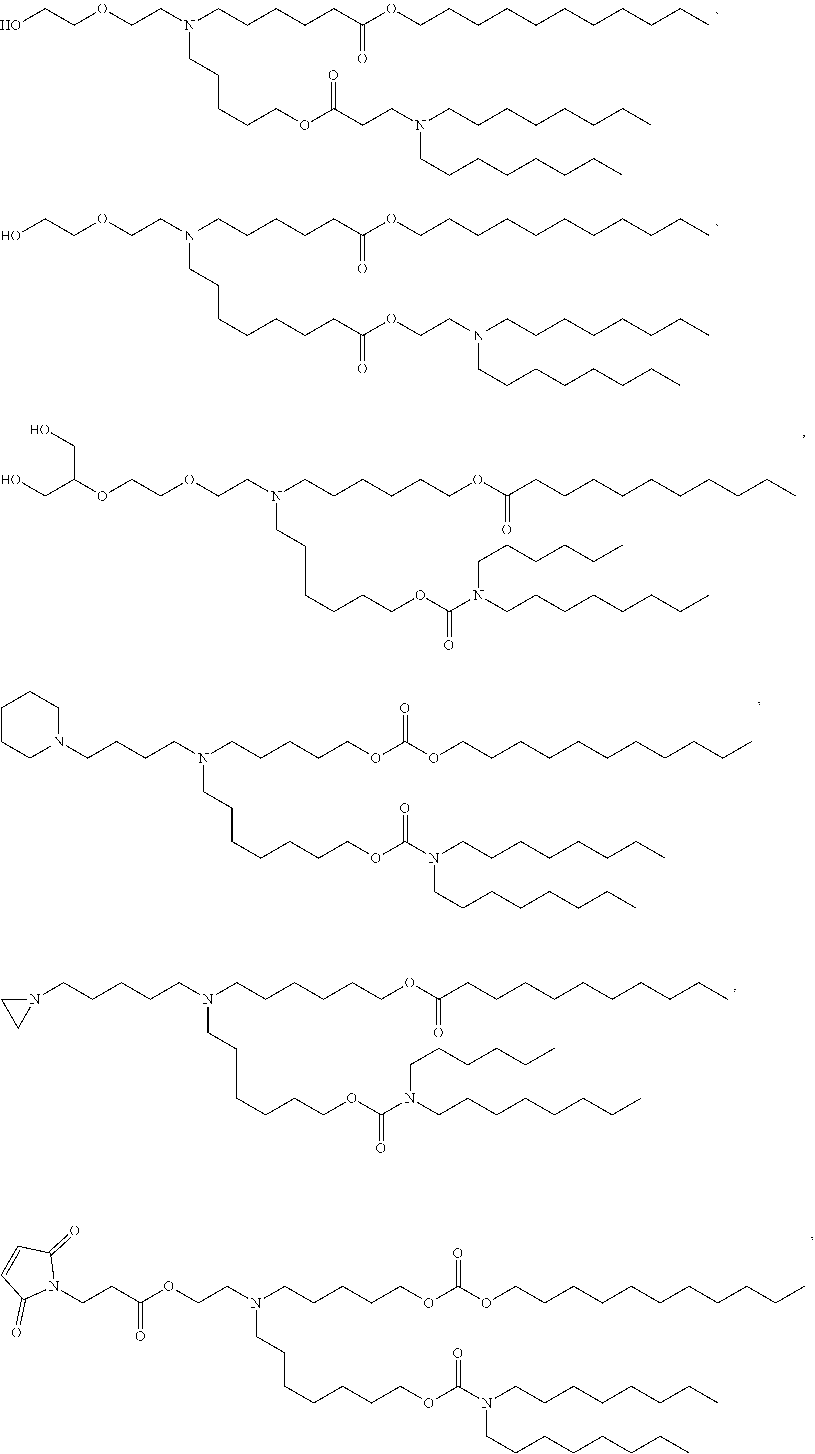

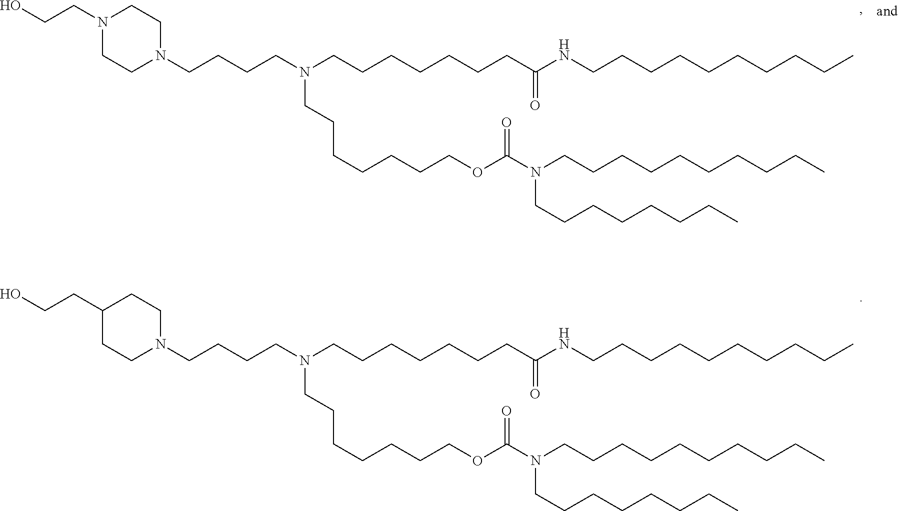 [SMILES: CCCCCCCCCCNC(=O)CCCCCCCN(CCCCCCCOC(=O)N(CCCCCCCC)CCCCCCCCCC)CCCCN1CCN(CCO)CC1 ; CCCCCCCCCCNC(=O)CCCCCCCN(CCCCCCCOC(=O)N(CCCCCCCC)CCCCCCCCCC)CCCCN1CCC(CCO)CC1]

12. A cationic liposome, wherein the cationic liposome contains any cationic lipid according to claim 1.

13. The cationic liposome according to claim 12, wherein said cationic liposome also contains one or more types of lipids selected from the group consisting of neutral lipid, steroid lipid, and PEGylated lipid; wherein, said neutral lipid is phospholipid.

14. The cationic liposome according to claim 13, wherein said neutral lipid is selected from the group consisting of 1,2-dilinoleoyl-sn-glycero-3-phosphocholines, 1,2-dimyristoleoyl-sn-glycero-3-phosphocholines, 1,2-dioleoyl-sn-glycero-3-phosphocholines, 1,2-dipalmitoyl-sn-glycero-3-phosphocholines, 1,2-distearoyl-sn-glycero-3-phosphatidylcholines, 1,2-diundecanoyl-sn-glycero-3-phosphatidylcholines, 1-plamitoyl-2-oleoyl-sn-glycero-3-phosphocholines, 1,2-di-O-octadecenyl-sn-glycero-3-phosphatidylcholines, 1-oleoyl-2-cholesterylhemisuccinyl-sn-glycero-3-phosphocholines, 1-hexadecyl-sn-glycero-3-phosphatidylcholines, 1,2-dilinolenoyl-sn-glycero-3-phosphatidylcholines, 1,2-diarachidonoyl-sn-glycero-3-phosphatidylcholines, 1,2-didecosahexaenoyl-sn-glycero-3-phosphocholines, 1,2-dioleoyl-sn-glycero-3-phosphoethanolamines, 1,2-diphytanyl-sn-glycero-3-phosphoethanolamines, 1,2-distearoyl-sn-glycero-3-phosphoethanolamines, 1,2-dilinoleoyl-sn-glycero-3-phosphoethanolamines, 1,2-dilinolenoyl-sn-glycero-3-phosphoethanolamines, 1,2-diarachidonoyl-sn-glycero-3-phosphoethanolamines, 1,2-didecosahexaenoyl-sn-glycero-3-phosphoethanolamines, 1,2-dioleoyl-sn-glycero-3-phospho-rac-(1-glycerol) sodium salts, dioleoyl phosphatidylserines, dipalmitoylphosphatidylglycerols, palmitoyloleoyl phosphatidylethanolamines, distearoyl phosphatidylethanolamines, dipalmitoyl phosphatidylethanolamines, dimyristoleoyl phosphoethanolamines, 1-stearoyl-2-oleoyl-stearoylethanolamines, 1-stearoyl-2-oleoyl-phosphatidylcholines, sphingomyelins, phosphatidylcholines, phosphatidylethnolamines, phosphatidylserines, phosphatidylinositols, phosphatidic acids, palmitoyloleoyl phosphatidylcholines, lysophosphatidylcholines, lysophosphatidylethanolamines, and combinations thereof.

15. The cationic liposome according to claim 13, wherein said steroid lipid is selected from the group consisting of a cholesterol, a coprostanol, a sitosterol, an ergosterol, a campesterol, a stigmasterol, a brassicasterol tomatidine, an ursolic acid, an α-tocopherol, and any mixture thereof.

16. The cationic liposome according to claim 13, wherein said PEGylated lipid is selected from the group consisting of 1,2-dimyristoyl-sn-glycerol methoxypolyethylene glycol, 1,2-distearoyl-sn-glycero-3-phosphoethanolamine-N-[amino(polyethylene glycol)], PEG-cholesterol, PEG-diacylglycamide, PEG-dialkyloxypropyl, and specifically PEG500-dipalmitoylphosphatidylcholine, PEG2000-dipalmitoylphosphatidylcholine, PEG500-distearylphosphatidylethanolamine PEG2000-distearylphosphatidylethanolamine, PEG500-1,2-dioleoylphosphatidylethanolamine, PEG2000-1,2-dioleoylphosphatidylethanolamine, and PEG2000-2,3-dimyristoylglycerol.

17. The cationic liposome according to claim 13, wherein said cationic liposome contains 20% to 80% cationic lipids, 5% to 15% neutral lipids, 25% to 55% steroid lipids, and 0.5% to 10% PEGylated lipids, said percentage is the molar percentage of each lipid relative to the total lipids in a solution containing the solvent.

18. The cationic liposome according to claim 13, wherein the molar percentage of said cationic lipids relative to the total lipids in a solution containing the solvent is 30% to 65%, and more specifically it is 35%, 40%, 45%, 46%, 47%, 48%, 49%, 50%, or 55%.

19. The cationic liposome according to claim 13, wherein the molar percentage of said neutral lipids relative to the total lipids in a solution containing the solvent is 7.5% to 13%, more specifically it is 8%, 9%, 10%, 11%, or 12%.

20. The cationic liposome according to claim 13, wherein the molar percentage of said steroid lipids relative to the total lipids in a solution containing the solvent is 35% to 50%, more specifically it is 40%, 41%, 42%, 43%, 44%, 45%, 46%, 47%, 48%, 49%, or 50%.

21. The cationic liposome according to claim 13, wherein the molar percentage of said PEGylated lipids relative to the total lipids in a solution containing the solvent is 0.5% to 5%, and more specifically it is 1.5%, 1.6%, 1.7%, 1.8%, or 1.9%.

22. A cationic liposome pharmaceutical composition, wherein the cationic liposome pharmaceutical composition contains the cationic liposome according to claim 13 and drugs; said drug is selected from the group consisting of nucleic acid drug, genetic vaccine, antitumor drug, small molecule drug, polypeptide drug, and protein drug.

23. The cationic liposome pharmaceutical composition according to claim 22, wherein said nucleic acid drug is selected from the group consisting of DNA, antisense nucleic acid, plasmid, mRNA, interfering nucleic acid, aptamer, antagomir, miRNA, ribozyme, and siRNA.

24. The cationic liposome pharmaceutical composition according to claim 23, wherein said pharmaceutical composition is used as a drug, selected from the group consisting of antitumor agents, antiviral agents, antifungal agents, and vaccines.

25. (canceled)
